(19) United States
(12) Patent Application Publication
(10) Pub. No.: US 2024/0066138 A1
(43) Pub. Date: Feb. 29, 2024

(54) ANTIBODY-DRUG CONJUGATES, PHARMACEUTICAL COMPOSITIONS, AND THERAPEUTIC APPLICATIONS

(71) Applicant: Shanghai Ruotuo Biosciences Co., Ltd., Shanghai (CN)

(72) Inventors: Hong Fu, Belmont, CA (US); Ziyang Zhong, Belmont, CA (US)

(21) Appl. No.: 18/258,209

(22) PCT Filed: Dec. 22, 2021
(86) PCT No.: PCT/US2021/064782

§ 371 Date: Jun. 18, 2023

Related U.S. Application Data
(60) Provisional application No. 63/129,845, filed on Dec. 23, 2020.

(51) Int. Cl. A61K 47/68; A61P 35/00; C07D 491/22; C07K 16/32

(52) U.S. Cl. CPC A61K 47/68037; A61P 35/00; C07D 491/22; C07K 16/32

(57) ABSTRACT

Provided herein are antibody-drug conjugates, for example, a compound of Formula (I), and pharmaceutical compositions thereof. Also provided herein are methods of their use for treating, preventing, or ameliorating one or more symptoms of a proliferative disease.

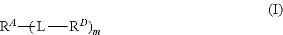

### CROSS REFERENCE TO RELATED APPLICATION

This application claims the benefit of the priority of U.S. Provisional Application No. 63/129,845, filed Dec. 23, 2020; the disclosure of which is incorporated herein by reference in its entirety.

### FIELD

Provided herein are antibody-drug conjugates and pharmaceutical compositions thereof. Also provided herein are methods of their use for treating, preventing, or ameliorating one or more symptoms of a proliferative disease.

### REFERENCE TO A SEQUENCE LISTING

The present specification is being filed with a Sequence Listing in Computer Readable Form (CRF), which is entitled 216A010WO01_SEQ_LIST_ST25.txt of 56,252 bytes in size and created Dec. 19, 2021; the content of which is incorporated herein by reference in its entirety.

### BACKGROUND

An antibody-drug conjugate (ADC) is a monoclonal antibody tethered to a cytotoxic drug (also known as a payload or warhead) via a chemical linker. Polakis, Pharmacol. Rev. 2016, 68, 3-19; Beck et al., Nat. Rev. Drug. Discov. 2017, 16, 315-37; Chau et al., Lancet 2019, 394, 793-804; Birrer et al., J. Natl. Cancer Inst. 2019, 111, 538-49. Thus, an ADC is a targeted biopharmaceutical drug that, via the specificity of its monoclonal antibody to a cancer cell surface antigen, delivers and releases its cytotoxic payload directly to targeted cancer cells, resulting in a greater potency and a broader therapeutic window as compared with the cytotoxic drug by itself. Polakis, Pharmacol. Rev. 2016, 68, 3-19; Chau et al., Lancet 2019, 394, 793-804. Mechanistically, an ADC exerts its biological functions by binding to a targeted cancer cell selectively, followed by the internalization of an ADC-antigen complex formed on the cell surface via clathrin-mediated endocytosis and the release of the cell-killing payload inside the targeted cancer cell. Chau et al., Lancet 2019, 394, 793-804.

Despite the advances in cancer treatment, cancer remains a major worldwide public health problem. It was estimated that there will be 1,806,590 new cancer cases diagnosed and 606,520 cancer deaths in the US alone in 2020. Cancer Facts & Figures. 2020. Cancer is the second-leading cause of death worldwide. Therefore, there is a need for an effective therapy for cancer treatment.

### SUMMARY OF THE DISCLOSURE

Provided herein is a compound of Formula (I):

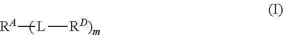

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
   R^{A} is an antibody or an antigen-binding fragment thereof;
   each L is independently a linker;
   m is an integer of 1, 2, 3, 4, 5, 6, 7, 8, 9, 10, 11, 12, 13, 14, 15, or 16; and
   each R^{D} is independently

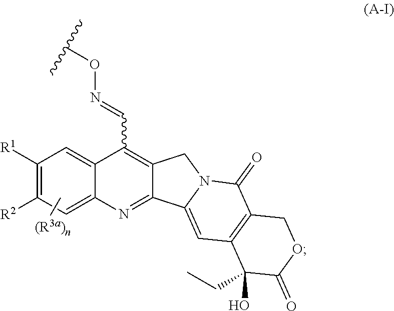

wherein:
   R¹ and R² are each independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1C}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c} or —S(O)₂NR^{1b}R^{1c}; each R^{3a} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c} or —S(O)₂NR^{1b}R^{1c};
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and
n is an integer of 0, 1, 2, 3, 4, 5, 6, 7, or 8;
wherein each alkyl, heteroalkyl, alkenyl, alkynyl, cycloalkyl, aryl, aralkyl, heteroaryl, and heterocyclyl is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{f}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e} C(O)NR^{f}R^{g}, —NR^{e}C(O)SR^{f}, —NR^{e} C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

Also provided herein is a pharmaceutical composition comprising a compound of Formula (I), or a diastereomer, a mixture of two or more diastereomers, a tautomer, a mixture of two or more tautomers, or an isotopic variant thereof; or a pharmaceutically acceptable salt, solvate, hydrate, or prodrug thereof; and a pharmaceutically acceptable excipient.

Additionally provided herein is a method of treating, preventing, or ameliorating one or more symptoms of a proliferative disease in a subject, comprising administering to the subject in need thereof a therapeutically effective amount of a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

Furthermore, provided herein is a method of inhibiting the growth of a cell, comprising contacting the cell with a compound of Formula (I), or an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug thereof.

Provided herein is a compound of Formula (IA):

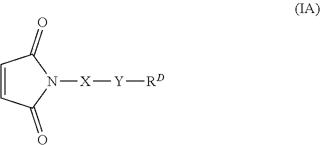

or a diastereomer, a mixture of two or more diastereomers, a tautomer, a mixture of two or more tautomers, or an isotopic variant thereof; or a pharmaceutically acceptable salt, solvate, or hydrate thereof; wherein:
   X is C₁₋₄₀ alkylene, C₁₋₄₀ heteroalkylene, C₂₋₄₀ alkenylene, C₂₋₄₀ heteroalkenylene, C₂₋₄₀ alkynylene, or C₂₋₄₀ heteroalkynylene, wherein one or more methylene groups are each independently and optionally replaced by a divalent group and each divalent group is independently C₃₋₁₀ cycloalkylene, C₆₋₁₄ arylene, heteroarylene, or heterocyclylene;
   Y is independently a bond, C₁₋₆ alkylene, C₁₋₆ heteroalkylene, C₂₋₆ alkenylene, C₂₋₆ alkynylene, C₃₋₁₀ cycloalkylene, C₆₋₁₄ arylene, C₇₋₁₅ aralkylene, heteroarylene, or heterocyclylene; and
   R^{D} is

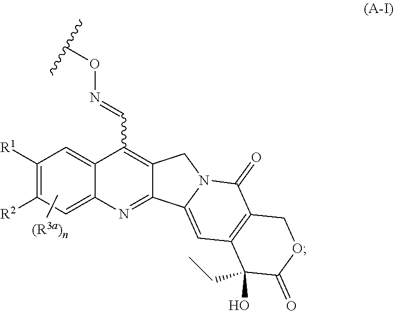

wherein:
R¹ and R² are each independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1C}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c} or —S(O)₂NR^{1b}R^{1c};
each R^{3a} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c} or —S(O)₂NR^{1b}R^{1c};
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and
n is an integer of 0, 1, 2, 3, 4, 5, 6, 7, or 8;
wherein each alkyl, alkylene, heteroalkyl, heteroalkylene, alkenyl, alkenylene, heteroalkenylene, alkynyl, alkynylene, heteroalkynylene, cycloalkyl, cycloalkylene, aryl, arylene, aralkyl, heteroaryl, heteroarylene, heterocyclyl, and heterocyclylene is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{f}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

### DETAILED DESCRIPTION

To facilitate understanding of the disclosure set forth herein, a number of terms are defined below.

Generally, the nomenclature used herein and the laboratory procedures in organic chemistry, medicinal chemistry, biochemistry, biology, immunology, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject. In one embodiment, the subject is a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “contacting” or “contact” is meant to refer to bringing together of a therapeutic agent and a biological molecule (e.g., a protein, enzyme, RNA, or DNA), cell, or tissue such that a physiological and/or chemical effect takes place as a result of such contact. Contacting can take place in vitro, ex vivo, or in vivo. In one embodiment, a therapeutic agent is contacted with a biological molecule in vitro to determine the effect of the therapeutic agent on the biological molecule. In another embodiment, a therapeutic agent is contacted with a cell in cell culture (in vitro) to determine the effect of the therapeutic agent on the cell. In yet another embodiment, the contacting of a therapeutic agent with a biological molecule, cell, or tissue includes the administration of a therapeutic agent to a subject having the biological molecule, cell, or tissue to be contacted.

The term “therapeutically effective amount” or “effective amount” is meant to include the amount of a compound that, when administered, is sufficient to prevent development of, or alleviate to some extent, one or more of the symptoms of the disorder, disease, or condition being treated. The term “therapeutically effective amount” or “effective amount” also refers to the amount of a compound that is sufficient to elicit a biological or medical response of a biological molecule (e.g., a protein, enzyme, RNA, or DNA), cell, tissue, system, animal, or human, which is being sought by a researcher, veterinarian, medical doctor, or clinician.

The term “IC₅₀” or “EC₅₀” refers to an amount, concentration, or dosage of a compound that is required for 50% inhibition of a maximal response in an assay that measures such a response.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of a subject (e.g., a human or an animal) without excessive toxicity, irritation, allergic response, immunogenicity, or other problems or complications, and commensurate with a reasonable benefit/risk ratio. See, e.g., Remington: The Science and Practice of Pharmacy, 22nd ed.; Allen Ed.; Pharmaceutical Press: London, 2012; Handbook of Pharmaceutical Excipients, 8th ed.; Sheskey et al., Eds.; Pharmaceutical Press: London, 2017; Handbook of Pharmaceutical Additives, 3rd ed.; Ash and Ash Eds.; Synapse Information Resources: 2007; Pharmaceutical Preformulation and Formulation, 2nd ed.; Gibson Ed.; Drugs and the Pharmaceutical Sciences 199; Informa Healthcare: New York, NY, 2009.

The term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 25%, 20%, 15%, 10%, 9%, 8%, 7%, 6%, 5%, 4%, 3%, 2%, 1%, 0.5%, or 0.05% of a given value or range.

The term “alkyl” refers to a linear or branched saturated monovalent hydrocarbon radical, wherein the alkyl is optionally substituted with one or more substituents Q as described herein. For example, C₁₋₆ alkyl refers to a linear saturated monovalent hydrocarbon radical of 1 to 6 carbon atoms or a branched saturated monovalent hydrocarbon radical of 3 to 6 carbon atoms. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms, e.g., n-propyl and isopropyl), butyl (including all isomeric forms, e.g., n-butyl, isobutyl, sec-butyl, and t-butyl), pentyl (including all isomeric forms, e.g., n-pentyl, isopentyl, sec-pentyl, neopentyl, and tert-pentyl), and hexyl (including all isomeric forms, e.g., n-hexyl, isohexyl, and sec-hexyl).

The term “heteroalkyl” refers to a linear or branched saturated monovalent hydrocarbon radical that contains one or more heteroatoms on its main chain, each independently selected from O, S, and N. The heteroalkyl is optionally substituted with one or more substituents Q as described herein. For example, C₁₋₆ heteroalkyl refers to a linear saturated monovalent hydrocarbon radical of 1 to 6 carbon atoms or a branched saturated monovalent hydrocarbon radical of 3 to 6 carbon atoms. In certain embodiments, the heteroalkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ heteroalkyl groups are also referred as “lower heteroalkyl.” Examples of heteroalkyl groups include, but are not limited to, —OCH₃, —OCH₂CH₃, —CH₂OCH₃, —NHCH₃, —ONHCH₃, —NHOCH₃, —SCH₃, —CH₂NHCH₂CH₃, and —NHCH₂CH₂CH₃. Examples of substituted heteroalkyl groups include, but are not limited to, —CH₂NHC(O)CH₃ and —NHC(O)CH₂CH₃.

The terms “alkylene” and “alkanediyl” are used interchangeably herein in reference to a linear or branched saturated divalent hydrocarbon radical, wherein the alkanediyl is optionally be substituted with one or more substituents Q as described herein. For example, C₁₋₆ alkanediyl refers to a linear saturated divalent hydrocarbon radical of 1 to 6 carbon atoms or a branched saturated divalent hydrocarbon radical of 3 to 6 carbon atoms. In certain embodiments, the alkanediyl is a linear saturated divalent hydrocarbon radical that has 1 to 30 (C₁₋₃₀), 1 to 20 (C₁₋₂₀), 1 to 15 (C₁₋₁₅), 1 to 10 (C₁₋₁₀), or 1 to 6 (C₁₋₆) carbon atoms, or branched saturated divalent hydrocarbon radical of 3 to 30 (C₃₋₃₀), 3 to 20 (C₃₋₂₀), 3 to 15 (C₃₋₁₅), 3 to 10 (C₃₋₁₀), or 3 to 6 (C₃₋₆) carbon atoms. As used herein, linear C₁_s and branched C₃₋₆ alkanediyl groups are also referred as “lower alkanediyl.” Examples of alkanediyl groups include, but are not limited to, methanediyl, ethanediyl (including all isomeric forms, e.g., ethane-1,1-diyl and ethane-1,2-diyl), propanediyl (including all isomeric forms, e.g., propane-1,1-diyl, propane-1,2-diyl, and propane-1,3-diyl), butanediyl (including all isomeric forms, e.g., butane-1,1-diyl, butane-1,2-diyl, butane-1,3-diyl, and butane-1,4-diyl), pentanediyl (including all isomeric forms, e.g., pentane-1,1-diyl, pentane-1,2-diyl, pentane-1,3-diyl, and pentane-1,5-diyl), and hexanediyl (including all isomeric forms, e.g., hexane-1,1-diyl, hexane-1,2-diyl, hexane-1,3-diyl, and hexane-1,6-diyl). Examples of substituted alkanediyl groups include, but are not limited to, —C(O)CH₂—, —C(O)(CH₂)₂—, —C(O)(CH₂)₃—, —C(O)(CH₂)₄—, —C(O)(CH₂)₅—, —C(O)(CH₂)₆—, —C(O)(CH₂)₇—, —C(O)(CH₂)₈—, —C(O)(CH₂)₉—, —C(O)(CH₂)₁₀—, —C(O)CH₂C(O)—, —C(O)(CH₂)₂C(O)—, —C(O)(CH₂)₃C(O)—, —C(O)(CH₂)₄C(O)—, or —C(O)(CH₂)₅C(O)—.

The terms “heteroalkylene” and “heteroalkanediyl” are used interchangeably herein in reference to a linear or branched saturated divalent hydrocarbon radical that contains one or more heteroatoms in its main chain, each independently selected from O, S, and N. The heteroalkylene is optionally substituted with one or more substituents Q as described herein. For example, C₁₋₆ heteroalkylene refers to a linear saturated divalent hydrocarbon radical of 1 to 6 carbon atoms or a branched saturated divalent hydrocarbon radical of 3 to 6 carbon atoms. In certain embodiments, the heteroalkylene is a linear saturated divalent hydrocarbon radical that has 1 to 20 (C₁-20), 1 to 15 (C₁₋₁₅), 1 to 10 (C₁₋₁₀), or 1 to 6 (C₁₋₆) carbon atoms, or branched saturated divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ heteroalkylene groups are also referred as “lower heteroalkylene.” Examples of heteroalkylene groups include, but are not limited to, —CH₂O—, —(CH₂)₂O—, —(CH₂)₃O—, —(CH₂)₄O—, —(CH₂)₅O—, —(CH₂)₆O—, —(CH₂)₇O—, —(CH₂)₈O—, —(CH₂)₉O—, —(CH₂)₁₀O—, —CH₂OCH₂—, —CH₂CH₂O—, —(CH₂CH₂O)₂—, —(CH₂CH₂O)₃—, —(CH₂CH₂O)₄—, —(CH₂CH₂O)₅—, —CH₂NH—, —CH₂NHCH₂—, —CH₂CH₂NH—, —CH₂S—, —CH₂SCH₂—, and —CH₂CH₂S—. Examples of substituted heteroalkylene groups include, but are not limited to, —C(O)CH₂O—, —C(O)(CH₂)₂O—, —C(O)(CH₂)₃O—, —C(O)(CH₂)₄O—, —C(O)(CH₂)₅O—, —C(O)(CH₂)₆O—, —C(O)(CH₂)₇O—, —C(O)(CH₂)₈O—, —C(O)(CH₂)₉O—, —C(O)(CH₂)₁₀O—, —C(O)CH₂OCH₂CH₂O—, —C(O)CH₂O(CH₂CH₂O)₂—, —C(O)CH₂O(CH₂CH₂O)₃—, —C(O)CH₂O(CH₂CH₂O)₄, —C(O)CH₂O(CH₂CH₂O)₅—, —CH₂NHC(O)CH₂—, or —CH₂CH₂C(O)NH—.

The term “alkenyl” refers to a linear or branched monovalent hydrocarbon radical, which contains one or more, in one embodiment, one, two, three, or four, in another embodiment, one, carbon-carbon double bond(s). The alkenyl is optionally substituted with one or more substituents Q as described herein. The term “alkenyl” embraces radicals having a “cis” or “trans” configuration or a mixture thereof, or alternatively, a “Z” or “E” configuration or a mixture thereof, as appreciated by those of ordinary skill in the art. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propenyl (including all isomeric forms, e.g., propen-1-yl, propen-2-yl, and allyl), and butenyl (including all isomeric forms, e.g., buten-1-yl, buten-2-yl, buten-3-yl, and 2-buten-1-yl).

The terms “alkenylene” and “alkenediyl” are used interchangeably herein in reference to a linear or branched divalent hydrocarbon radical, which contains one or more, in one embodiment, one, two, three, or four, in another embodiment, one, carbon-carbon double bond(s). The alkenediyl is optionally substituted with one or more substituents Q as described herein. The term “alkenediyl” embraces radicals having a “cis” or “trans” configuration or a mixture thereof, or alternatively, a “Z” or “E” configuration or a mixture thereof, as appreciated by those of ordinary skill in the art. For example, C₂₋₆ alkenediyl refers to a linear unsaturated divalent hydrocarbon radical of 2 to 6 carbon atoms or a branched unsaturated divalent hydrocarbon radical of 3 to 6 carbon atoms. In certain embodiments, the alkenediyl is a linear divalent hydrocarbon radical of 2 to 30 (C₂₋₃₀), 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 3 to 30 (C₃₋₃₀), 3 to 20 (C₃₋₂₀), 3 to 15 (C₃₋₁₅), 3 to 10 (C₃₋₁₀), or 3 to 6 (C₃₋₆) carbon atoms. Examples of alkenediyl groups include, but are not limited to, ethenediyl (including all isomeric forms, e.g., ethene-1,1-diyl and ethene-1,2-diyl), propenediyl (including all isomeric forms, e.g., 1-propene-1,1-diyl, 1-propene-1,2-diyl, and 1-propene-1,3-diyl), butenediyl (including all isomeric forms, e.g., 1-butene-1,1-diyl, 1-butene-1,2-diyl, and 1-butene-1,4-diyl), pentenediyl (including all isomeric forms, e.g., 1-pentene-1,1-diyl, 1-pentene-1,2-diyl, and 1-pentene-1,5-diyl), and hexenediyl (including all isomeric forms, e.g., 1-hexene-1,1-diyl, 1-hexene-1,2-diyl, 1-hexene-1,3-diyl, 1-hexene-1,4-diyl, 1-hexene-1,5-diyl, and 1-hexene-1,6-diyl).

The terms “heteroalkenylene” and “heteroalkenediyl” are used interchangeably herein in reference to a linear or branched divalent hydrocarbon radical, which contains one or more, in one embodiment, one, two, three, or four, in another embodiment, one, carbon-carbon double bond(s), and which contains one or more heteroatoms each independently selected from O, S, and N in the hydrocarbon chain. The heteroalkenylene is optionally substituted with one or more substituents Q as described herein. The term “heteroalkenylene” embraces radicals having a “cis” or “trans” configuration or a mixture thereof, or alternatively, a “Z” or “E” configuration or a mixture thereof, as appreciated by those of ordinary skill in the art. For example, C₂₋₆ heteroalkenylene refers to a linear unsaturated divalent hydrocarbon radical of 2 to 6 carbon atoms or a branched unsaturated divalent hydrocarbon radical of 3 to 6 carbon atoms. In certain embodiments, the heteroalkenylene is a linear divalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of heteroalkenylene groups include, but are not limited to, —CH═CHO—, —CH═CHOCH₂—, —CH═CHCH₂O—, —CH═CHS—, —CH═CHSCH₂—, —CH═CHCH₂S—, or —CH═CHCH₂NH—.

The term “alkynyl” refers to a linear or branched monovalent hydrocarbon radical, which contains one or more, in one embodiment, one, two, three, or four, in another embodiment, one, carbon-carbon triple bond(s). The alkynyl is optionally substituted with one or more substituents Q as described herein. For example, C₂₋₆ alkynyl refers to a linear unsaturated monovalent hydrocarbon radical of 2 to 6 carbon atoms or a branched unsaturated monovalent hydrocarbon radical of 4 to 6 carbon atoms. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 4 to 20 (C₄₋₂₀), 4 to 15 (C₄₋₁₅), 4 to 10 (C₄₋₁₀), or 4 to 6 (C₄₋₆) carbon atoms. Examples of alkynyl groups include, but are not limited to, ethynyl (—C≡CH), propynyl (including all isomeric forms, e.g., 1-propynyl (—C≡CCH₃) and propargyl (—CH₂C≡CH)), butynyl (including all isomeric forms, e.g., 1-butyn-1-yl and 2-butyn-1-yl), pentynyl (including all isomeric forms, e.g., 1-pentyn-1-yl and 1-methyl-2-butyn-1-yl), and hexynyl (including all isomeric forms, e.g., 1-hexyn-1-yl and 2-hexyn-1-yl).

The terms “alkynylene” and “alkynediyl” are used interchangeably herein in reference to a linear or branched divalent hydrocarbon radical, which contains one or more, in one embodiment, one, two, three, or four, in another embodiment, one, carbon-carbon triple bond(s). The alkynediyl is optionally substituted with one or more substituents Q as described herein. For example, C₂₋₆ alkynediyl refers to a linear unsaturated divalent hydrocarbon radical of 2 to 6 carbon atoms or a branched unsaturated divalent hydrocarbon radical of 4 to 6 carbon atoms. In certain embodiments, the alkynediyl is a linear divalent hydrocarbon radical of 2 to 30 (C₂₋₃₀), 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 4 to 30 (C₄₋₃₀), 4 to 20 (C₄₋₂₀), 4 to 15 (C₄₋₁₅), 4 to 10 (C₄₋₁₀), or 4 to 6 (C₄₋₆) carbon atoms. Examples of alkynediyl groups include, but are not limited to, ethynediyl, propynediyl (including all isomeric forms, e.g., 1-propyne-1,3-diyl and 1-propyne-3,3-diyl), butynediyl (including all isomeric forms, e.g., 1-butyne-1,3-diyl, 1-butyne-1,4-diyl, and 2-butyne-1,1-diyl), pentynediyl (including all isomeric forms, e.g., 1-pentyne-1,3-diyl, 1-pentyne-1,4-diyl, and 2-pentyne-1,1-diyl), and hexynediyl (including all isomeric forms, e.g., 1-hexyne-1,3-diyl, 1-hexyne-1,4-diyl, and 2-hexyne-1,1-diyl).

The terms “heteroalkynylene” and “heteroalkynediyl” are used interchangeably herein in reference to a linear or branched divalent hydrocarbon radical, which contains one or more, in one embodiment, one, two, three, or four, in another embodiment, one, carbon-carbon triple bond(s), and which contains one or more heteroatoms in its main chain, each independently selected from O, S, and N. The heteroalkynylene is optionally substituted with one or more substituents Q as described herein. For example, C₂₋₆ heteroalkynylene refers to a linear unsaturated divalent hydrocarbon radical of 2 to 6 carbon atoms or a branched unsaturated divalent hydrocarbon radical of 4 to 6 carbon atoms. In certain embodiments, the heteroalkynylene is a linear divalent hydrocarbon radical of 2 to 30 (C₂₋₃₀), 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 4 to 30 (C₄₋₃₀), 4 to 20 (C₄₋₂₀), 4 to 15 (C₄₋₁₅), 4 to 10 (C₄₋₁₀), or 4 to 6 (C₄₋₆) carbon atoms. Examples of heteroalkynylene groups include, but are not limited to, —C≡CCH₂O—, —C≡CCH₂S—, or —C≡CCH₂NH—.

The term “cycloalkyl” refers to a cyclic monovalent hydrocarbon radical, which is optionally substituted with one or more substituents Q as described herein. In one embodiment, the cycloalkyl is a saturated or unsaturated but non-aromatic, and/or bridged or non-bridged, and/or fused bicyclic group. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. In one embodiment, the cycloalkyl is monocyclic. In another embodiment, the cycloalkyl is bicyclic. In yet another embodiment, the cycloalkyl is tricyclic. In still another embodiment, the cycloalkyl is polycyclic. Examples of cycloalkyl groups include, but are not limited to, cyclopropyl, cyclobutyl, cyclopentyl, cyclopentenyl, cyclohexyl, cyclohexenyl, cyclohexadienyl, cycloheptyl, cycloheptenyl, bicyclo[1.1.1]pentyl, bicyclo[2.1.1]hexyl, bicyclo[2.2.1]heptyl, bicyclo[2.2.2]octyl, decalinyl, and adamantyl.

The terms “cycloalkylene” and “cycloalkanediyl” are used interchangeably herein in reference to a cyclic divalent hydrocarbon radical, which may be optionally substituted with one or more substituents Q as described herein. In one embodiment, cycloalkanediyl groups may be saturated or unsaturated but non-aromatic, and/or bridged, and/or non-bridged, and/or fused bicyclic groups. In certain embodiments, the cycloalkanediyl has from 3 to 30 (C₃₋₃₀), 3 to 20 (C₃₋₂₀), from 3 to 15 (C₃₋₁₅), from 3 to 10 (C₃₋₁₀), or from 3 to 7 (C₃₋₇) carbon atoms. Examples of cycloalkanediyl groups include, but are not limited to, cyclopropanediyl (including all isomeric forms, e.g., cyclopropane-1,1-diyl and cyclopropane-1,2-diyl), cyclobutanediyl (including all isomeric forms, e.g., cyclobutane-1,1-diyl, cyclobutane-1,2-diyl, and cyclobutane-1,3-diyl), cyclopentanediyl (including all isomeric forms, e.g., cyclopentane-1,1-diyl, cyclopentane-1,2-diyl, and cyclopentane-1,3-diyl), cyclohexanediyl (including all isomeric forms, e.g., cyclohexane-1,1-diyl, cyclohexane-1,2-diyl, cyclohexane-1,3-diyl, and cyclohex-1,4-diyl), cycloheptanediyl (including all isomeric forms, e.g., cycloheptane-1,1-diyl, cycloheptane-1,2-diyl, cycloheptane-1,3-diyl, and cycloheptane-1,4-diyl), decalinediyl (including all isomeric forms, e.g., decaline-1,1-diyl, decaline-1,2-diyl, and decaline-1,8-diyl), and adamantdiyl (including all isomeric forms, e.g., adamant-1,2-diyl, adamant-1,3-diyl, and adamant-1,8-diyl).

The term “aryl” refers to a monovalent monocyclic aromatic hydrocarbon radical and/or monovalent polycyclic aromatic hydrocarbon radical that contain at least one aromatic carbon ring. In certain embodiments, the aryl has from 6 to 20 (C₆₋₂₀), from 6 to 15 (C₆₋₁₅), or from 6 to 10 (C₆₋₁₀) ring carbon atoms. Examples of aryl groups include, but are not limited to, phenyl, naphthyl, fluorenyl, azulenyl, anthryl, phenanthryl, pyrenyl, biphenyl, and terphenyl. The aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In one embodiment, the aryl is monocyclic. In another embodiment, the aryl is bicyclic. In yet another embodiment, the aryl is tricyclic. In still another embodiment, the aryl is polycyclic. In certain embodiments, the aryl is optionally substituted with one or more substituents Q as described herein.

The terms “arylene” and “arenediyl” are used interchangeably herein in reference to a divalent monocyclic aromatic hydrocarbon radical or divalent polycyclic aromatic hydrocarbon radical that contains at least one aromatic hydrocarbon ring. In certain embodiments, the arylene has from 6 to 20 (C₆₋₂₀), from 6 to 15 (C₆₋₁₅), or from 6 to 10 (C₆₋₁₀) ring atoms. Examples of arylene groups include, but are not limited to, phenylene (including all isomeric forms, e.g., phen-1,2-diyl, phen-1,3-diyl, and phen-1,4-diyl), naphthylene (including all isomeric forms, e.g., naphth-1,2-diyl, naphth-1,3-diyl, and naphth-1,8-diyl), fluorenylene (including all isomeric forms, e.g., fluoren-1,2-diyl, fluoren-1,3-diyl, and fluoren-1,8-diyl), azulenylene (including all isomeric forms, e.g., azulen-1,2-diyl, azulen-1,3-diyl, and azulen-1,8-diyl), anthrylene (including all isomeric forms, e.g., anthr-1,2-diyl, anthr-1,3-diyl, and anthr-1,8-diyl), phenanthrylene (including all isomeric forms, e.g., phenanthr-1,2-diyl, phenanthr-1,3-diyl, and phenanthr-1,8-diyl), pyrenylene (including all isomeric forms, e.g., pyren-1,2-diyl, pyren-1,3-diyl, and pyren-1,8-diyl), biphenylene (including all isomeric forms, e.g., biphen-2,3-diyl, biphen-3,4′-diyl, and biphen-4,4′-diyl), and terphenylene (including all isomeric forms, e.g., terphen-2,3-diyl, terphen-3,4′-diyl, and terphen-4,4′-diyl). Arylene also refers to bicyclic or tricyclic carbon rings, where one of the rings is aromatic and the others of which may be saturated, partially unsaturated, or aromatic, for example, dihydronaphthylene (including all isomeric forms, e.g., dihydronaphth-1,2-diyl and dihydronaphth-1,8-diyl), indenylene (including all isomeric forms, e.g., inden-1,2-diyl, inden-1,5-diyl, and inden-1,7-diyl), indanylene (including all isomeric forms, e.g., indan-1,2-diyl, indan-1,5-diyl, and indan-1,7-diyl), or tetrahydronaphthylene (tetralinylene) (including all isomeric forms, e.g., tetrahydronaphth-1,2-diyl, tetrahydronaphth-1,5-diyl, and tetrahydronaphth-1,8-diyl). In certain embodiments, arylene is optionally substituted with one or more substituents Q as described herein.

The term “aralkyl” or “arylalkyl” refers to a monovalent alkyl group substituted with one or more aryl groups. In certain embodiments, the aralkyl has from 7 to 30 (C₇₋₃₀), from 7 to 20 (C₇₋₂₀), or from 7 to 16 (C₇₋₁₆) carbon atoms. Examples of aralkyl groups include, but are not limited to, benzyl, phenylethyl (including all isomeric forms, e.g., 1-phenylethyl and 2-phenylethyl), and phenylpropyl (including all isomeric forms, e.g., 1-phenylpropyl, 2-phenylpropyl, and 3-phenylpropyl). In certain embodiments, the aralkyl is optionally substituted with one or more substituents Q as described herein.

The term “aralkylene” or “arylalkylene” refers to a divalent alkyl group substituted with one or more aryl groups. In certain embodiments, the aralkylene has from 7 to 30 (C₇₋₃₀), from 7 to 20 (C₇₋₂₀), or from 7 to 16 (C₇₋₁₆) carbon atoms. Examples of aralkylene groups include, but are not limited to, benzylene (including all isomeric forms, e.g., phenylmethdiyl), phenylethylene (including all isomeric forms, e.g., 2-phenyl-ethan-1,1-diyl and 2-phenyl-ethan-1,2-diyl), and phenylpropylene (including all isomeric forms, e.g., 3-phenyl-propan-1,1-diyl, 3-phenyl-propan-1,2-diyl, and 3-phenyl-propan-1,3-diyl). In certain embodiments, the aralkylene is optionally substituted with one or more substituents Q as described herein.

The term “heteroaryl” refers to a monovalent monocyclic aromatic group or monovalent polycyclic aromatic group that contain at least one aromatic ring, wherein at least one aromatic ring contains one or more heteroatoms, each independently selected from O, S, and N, in the ring. The heteroaryl is bonded to the rest of a molecule through the aromatic ring. Each ring of a heteroaryl group can contain one or two O atoms, one or two S atoms, and/or one to four N atoms; provided that the total number of heteroatoms in each ring is four or less and each ring contains at least one carbon atom. In certain embodiments, the heteroaryl has from 5 to 20, from 5 to 15, or from 5 to 10 ring atoms. In one embodiment, the heteroaryl is monocyclic. Examples of monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. In another embodiment, the heteroaryl is bicyclic. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyrindyl (including all isomeric forms, e.g., furo[2,3-b]pyridinyl, furo[2,3-c]pyridinyl, furo[3,2-b]pyridinyl, furo[3,2-c]pyridinyl, furo[3,4-b]pyridinyl, and furo[3,4-c]pyridinyl), imidazopyridinyl (including all isomeric forms, e.g., imidazo[1,2-a]pyridinyl, imidazo[4,5-b]pyridinyl, and imidazo[4,5-c]pyridinyl), imidazothiazolyl (including all isomeric forms, e.g., imidazo[2,1-b]thiazolyl and imidazo[4,5-d]thiazolyl), indazolyl, indolizinyl, indolyl, isobenzofuranyl, isobenzothienyl (i.e., benzo[c]thienyl), isoindolyl, isoquinolinyl, naphthyridinyl (including all isomeric forms, e.g., 1,5-naphthyridinyl, 1,6-naphthyridinyl, 1,7-naphthyridinyl, and 1,8-naphthyridinyl), oxazolopyridinyl (including all isomeric forms, e.g., oxazolo[4,5-b]pyridinyl, oxazolo[4,5-c]pyridinyl, oxazolo[5,4-b]pyridinyl, and oxazolo[5,4-c]pyridinyl), phthalazinyl, pteridinyl, purinyl, pyrrolopyridyl (including all isomeric forms, e.g., pyrrolo[2,3-b]pyridinyl, pyrrolo[2,3-c]pyridinyl, pyrrolo[3,2-b]pyridinyl, and pyrrolo[3,2-c]pyridinyl), quinolinyl, quinoxalinyl, quinazolinyl, thiadiazolopyrimidyl (including all isomeric forms, e.g., [1,2,5]thiadiazolo[3,4-d]pyrimidinyl and [1,2,3]thiadiazolo[4,5-d]pyrimidinyl), and thienopyridyl (including all isomeric forms, e.g., thieno[2,3-b]pyridinyl, thieno[2,3-c]pyridinyl, thieno[3,2-b]pyridinyl, and thieno[3,2-c]pyridinyl). In yet another embodiment, the heteroaryl is tricyclic. Examples of tricyclic heteroaryl groups include, but are not limited to, acridinyl, benzindolyl, carbazolyl, dibenzofuranyl, perimidinyl, phenanthrolinyl, phenanthridinyl (including all isomeric forms, e.g., 1,5-phenanthrolinyl, 1,6-phenanthrolinyl, 1,7-phenanthrolinyl, 1,9-phenanthrolinyl, and 2,10-phenanthrolinyl), phenarsazinyl, phenazinyl, phenothiazinyl, phenoxazinyl, and xanthenyl. In certain embodiments, the heteroaryl is optionally substituted with one or more substituents Q as described herein.

The terms “heteroarylene” and “heteroarenediyl” are used interchangeably herein in reference to a divalent monocyclic aromatic group or divalent polycyclic aromatic group that contains at least one aromatic ring, wherein at least one aromatic ring contains one or more heteroatoms in the ring, each of which is independently selected from O, S, and N. A heteroarylene group has at least one linkage to the rest of a molecule via its aromatic ring(s). Each ring of a heteroarylene group can contain one or two O atoms, one or two S atoms, and/or one to four N atoms, provided that the total number of heteroatoms in each ring is four or less and each ring contains at least one carbon atom. In certain embodiments, the heteroarylene has from 5 to 20, from 5 to 15, or from 5 to 10 ring atoms. Examples of monocyclic heteroarylene groups include, but are not limited to, furandiyl, imidazoldiyl, isothiazoldiyl, isoxazoldiyl, oxadiazoldiyl, oxazoldiyl, pyrazindiyl, pyrazoldiyl, pyridazindiyl, pyridindiyl, pyrimidindiyl, pyrroldiyl, thiadiazoldiyl, thiazoldiyl, thiendiyl, tetrazoldiyl, triazinediyl, and triazoldiyl. Examples of bicyclic heteroarylene groups include, but are not limited to, benzofurandiyl, benzimidazoldiyl, benzoisoxazoldiyl, benzopyrandiyl, benzothiadiazoldiyl, benzothiazoldiyl, benzothiendiyl, benzotriazoldiyl, benzoxazoldiyl, furopyridindiyl (including all isomeric forms, e.g., furo[2,3-b]pyridindiyl, furo[2,3-c]pyridindiyl, furo[3,2-b]pyridindiyl, furo[3,2-c]pyridindiyl, furo[3,4-b]pyridindiyl, and furo[3,4-c]pyridindiyl), imidazopyridindiyl (including all isomeric forms, e.g., imidazo[1,2-a]pyridindiyl, imidazo[4,5-b]pyridindiyl, and imidazo[4,5-c]pyridindiyl), imidazothiazoldiyl (including all isomeric forms, e.g., imidazo[2,1-b]thiazoldiyl and imidazo[4,5-d]thiazoldiyl), indazoldiyl, indolizindiyl, indoldiyl, isobenzofurandiyl, isobenzothiendiyl (i.e., benzo[c]thiendiyl), isoindoldiyl, isoquinolindiyl, naphthyridindiyl (including all isomeric forms, e.g., 1,5-naphthyridindiyl, 1,6-naphthyridindiyl, 1,7-naphthyridindiyl, and 1,8-naphthyridindiyl), oxazolopyridindiyl (including all isomeric forms, e.g., oxazolo[4,5-b]pyridindiyl, oxazolo[4,5-c]pyridindiyl, oxazolo[5,4-b]pyridindiyl, and oxazolo[5,4-c]pyridindiyl), phthalazindiyl, pteridindiyl, purindiyl, pyrrolopyridindiyl (including all isomeric forms, e.g., pyrrolo[2,3-b]pyridindiyl, pyrrolo[2,3-c]pyridindiyl, pyrrolo[3,2-b]pyridindiyl, and pyrrolo[3,2-c]pyridindiyl), quinolindiyl, quinoxalindiyl, quinazolindiyl, thiadiazolopyrimidindiyl (including all isomeric forms, e.g., [1,2,5]thiadiazolo[3,4-d]pyrimidindiyl and [1,2,3]thiadiazolo[4,5-d]pyrimidindiyl), and thienopyridindiyl (including all isomeric forms, e.g., thieno[2,3-b]pyridindiyl, thieno[2,3-c]pyridindiyl, thieno[3,2-b]pyridindiyl, and thieno[3,2-c]pyridindiyl). Examples of tricyclic heteroarylene groups include, but are not limited to, acridindiyl, benzindoldiyl, carbazoldiyl, dibenzofurandiyl, perimidindiyl, phenanthrolindiyl (including all isomeric forms, e.g., 1,5-phenanthrolindiyl, 1,6-phenanthrolindiyl, 1,7-phenanthrolindiyl, 1,9-phenanthrolindiyl, and 2,10-phenanthrolindiyl), phenanthridindiyl, phenarsazindiyl, phenazindiyl, phenothiazindiyl, phenoxazindiyl, and xanthendiyl. In certain embodiments, heteroarylene is optionally substituted with one or more substituents Q as described herein.

The term “heterocyclyl” or “heterocyclic” refers to a monovalent monocyclic non-aromatic ring system or monovalent polycyclic ring system that contains at least one non-aromatic ring, wherein one or more of the non-aromatic ring atoms are heteroatoms, each independently selected from O, S, and N; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. The heterocyclyl is bonded to the rest of a molecule through the non-aromatic ring. In certain embodiments, the heterocyclyl is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclyl may be attached to the main structure at any heteroatom or carbon atom which results in the creation of a stable compound. Examples of heterocyclyls and heterocyclic groups include, but are not limited to, azepinyl, benzodioxanyl, benzodioxolyl, benzofuranonyl, chromanyl, decahydroisoquinolinyl, dihydrobenzofuranyl, dihydrobenzisothiazolyl, dihydrobenzisoxazinyl (including all isomeric forms, e.g., 1,4-dihydrobenzo[d][1,3]oxazinyl, 3,4-dihydrobenzo[c][1,2]-oxazinyl, and 3,4-dihydrobenzo[d][1,2]oxazinyl), dihydrobenzothienyl, dihydroisobenzofuranyl, dihydrobenzo[c]thienyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isochromanyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, thiochromanyl, tetrahydroquinolinyl, and 1,3,5-trithianyl. In certain embodiments, the heterocyclyl is optionally substituted with one or more substituents Q as described herein.

The term “heterocyclylene” refers to a divalent monocyclic non-aromatic ring system or divalent polycyclic ring system that contains at least one non-aromatic ring, wherein one or more of the non-aromatic ring atoms are heteroatoms independently selected from O, S, and N; and the remaining ring atoms are carbon atoms. Heterocyclylene groups are bonded to the rest of a molecule through the non-aromatic ring. In certain embodiments, the heterocyclylene group has from 3 to 20, from 3 to 15, from 3 to 10, from 3 to 8, from 4 to 7, or from 5 to 6 ring atoms. In certain embodiments, the heterocyclylene is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclylene may be attached to the main structure at any heteroatom or carbon atom which results in the creation of a stable compound. Examples of such heterocyclylene groups include, but are not limited to, azepindiyl, benzodioxandiyl, benzodioxoldiyl, benzofuranondiyl, chromandiyl, decahydroisoquinolindiyl, dihydrobenzofurandiyl, dihydrobenzisothiazoldiyl, dihydrobenzisoxazindiyl (including all isomeric forms, e.g., 1,4-dihydrobenzo[d][1,3]oxazindiyl, 3,4-dihydrobenzo[c][1,2]oxazindiyl, and 3,4-dihydrobenzo[d][1,2]oxazindiyl), dihydrobenzothiendiyl, dihydroisobenzofurandiyl, dihydrobenzo[c]thiendiyl, dihydrofurdiyl, dihydroisoindoldiyl, dihydropyrandiyl, dihydropyrazoldiyl, dihydropyrazindiyl, dihydropyridindiyl, dihydropyrimidindiyl, dihydropyrroldiyl, dioxolandiyl, 1,4-dithiandiyl, furanondiyl, imidazolidindiyl, imidazolindiyl, indolindiyl, isochromandiyl, isoindolindiyl, isothiazolidindiyl, isoxazolidindiyl, morpholindiyl, octahydroindoldiyl, octahydroisoindoldiyl, oxazolidinondiyl, oxazolidindiyl, oxirandiyl, piperazindiyl, piperidindiyl, 4-piperidondiyl, pyrazolidindiyl, pyrazolindiyl, pyrrolidindiyl, pyrrolindiyl, quinuclidindiyl, tetrahydrofurdiyl, tetrahydroisoquinolindiyl, tetrahydropyrandiyl, tetrahydrothiendiyl, thiamorpholindiyl, thiazolidindiyl, thiochromandiyl, tetrahydroquinolindiyl, and 1,3,5-trithiandiyl. In certain embodiments, the heterocyclylene is optionally substituted with one or more substituents Q as described herein.

The term “halogen,” “halide,” or “halo” refers to fluoro, chloro, bromo, and/or iodo.

The term “optionally substituted” is intended to mean that a group or substituent, such as an alkyl, heteroalkyl, alkylene, heteroalkylene, alkenyl, alkenylene, heteroalkenylene, alkynyl, alkynylene, heteroalkynylene, cycloalkyl, cycloalkylene, aryl, arylene, aralkyl, aralkylene, heteroaryl, heteroarylene, heterocyclyl, or heterocyclylene group, may be substituted with one or more, in one embodiment, one, two, three, or four, substituents Q, each of which is independently selected from, e.g., (a) deuterium (-D), cyano (—CN), halo, imino (═NH), nitro (—NO₂), and oxo (═O); (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{f}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a}. As used herein, all groups that can be substituted are “optionally substituted.”

In one embodiment, each Q^{a} is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, “optically active” and “enantiomerically active” refer to a collection of molecules, which has an enantiomeric excess of no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, an optically active compound comprises about 95% or more of one enantiomer and about 5% or less of the other enantiomer based on the total weight of the enantiomeric mixture in question. In certain embodiments, an optically active compound comprises about 98% or more of one enantiomer and about 2% or less of the other enantiomer based on the total weight of the enantiomeric mixture in question. In certain embodiments, an optically active compound comprises about 99% or more of one enantiomer and about 1% or less of the other enantiomer based on the total weight of the enantiomeric mixture in question.

In describing an optically active compound, the prefixes R and S are used to denote the absolute configuration of the compound about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the compound, R and S.

The term “isotopically enriched” refers to a compound that contains an unnatural proportion of an isotope at one or more of the atoms that constitute such a compound. In certain embodiments, an isotopically enriched compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an isotopically enriched compound is in a stable form, that is, non-radioactive. In certain embodiments, an isotopically enriched compound contains unnatural proportions of one or more isotopes, including, but not limited to, hydrogen (¹H), deuterium (2H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an isotopically enriched compound is in an unstable form, that is, radioactive. In certain embodiments, an isotopically enriched compound contains unnatural proportions of one or more isotopes, including, but not limited to, tritium (3H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I). It will be understood that, in a compound as provided herein, any hydrogen can be ²H, as example, or any carbon can be ¹³C, as example, or any nitrogen can be ¹⁵N, as example, or any oxygen can be ¹⁸O, as example, where feasible according to the judgment of one of ordinary skill in the art.

The term “isotopic enrichment” refers to the percentage of incorporation of a less prevalent isotope (e.g., D for deuterium or hydrogen-2) of an element at a given position in a molecule in the place of a more prevalent isotope (e.g., ¹H for protium or hydrogen-1) of the element. As used herein, when an atom at a particular position in a molecule is designated as a particular less prevalent isotope, it is understood that the abundance of that isotope at that position is substantially greater than its natural abundance.

The term “isotopic enrichment factor” refers the ratio between the isotopic abundance in an isotopically enriched compound and the natural abundance of a specific isotope.

The term “hydrogen” or the symbol “H” refers to the composition of naturally occurring hydrogen isotopes, which include protium (¹H), deuterium (2H or D), and tritium (3H), in their natural abundances. Protium is the most common hydrogen isotope having a natural abundance of more than 99.98%. Deuterium is a less prevalent hydrogen isotope having a natural abundance of about 0.0156%.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156% on average, deuterium enrichment at any position in a compound synthesized using non-enriched starting materials is about 0.0156% on average. As used herein, when a particular position in an isotopically enriched compound is designated as having deuterium, it is understood that the abundance of deuterium at that position in the compound is substantially greater than its natural abundance (0.0156%).

The term “carbon” or the symbol “C” refers to the composition of naturally occurring carbon isotopes, which include carbon-12 (¹²C) and carbon-13 (¹³C) in their natural abundances. Carbon-12 is the most common carbon isotope having a natural abundance of more than 98.89%. Carbon-13 is a less prevalent carbon isotope having a natural abundance of about 1.11%.

The term “carbon-13 enrichment” or “¹³C enrichment” refers to the percentage of incorporation of carbon-13 at a given position in a molecule in the place of carbon. For example, carbon-13 enrichment of 10% at a given position means that 10% of molecules in a given sample contain carbon-13 at the specified position. Because the naturally occurring distribution of carbon-13 is about 1.11% on average, carbon-13 enrichment at any position in a compound synthesized using non-enriched starting materials is about 1.11% on average. As used herein, when a particular position in an isotopically enriched compound is designated as having carbon-13, it is understood that the abundance of carbon-13 at that position in the compound is substantially greater than its natural abundance (1.11%).

The terms “substantially pure” and “substantially homogeneous” mean, when referred to a substance, sufficiently homogeneous to appear free of readily detectable impurities as determined by a standard analytical method used by one of ordinary skill in the art, including, but not limited to, thin layer chromatography (TLC), gel electrophoresis, high performance liquid chromatography (HPLC), gas chromatography (GC), nuclear magnetic resonance (NMR), and mass spectrometry (MS); or sufficiently pure such that further purification would not detectably alter the physical, chemical, biological, and/or pharmacological properties, such as enzymatic and biological activities, of the substance. In certain embodiments, “substantially pure” or “substantially homogeneous” refers to a collection of molecules, wherein at least about 95%, at least about 96%, at least about 97%, at least about 98%, at least about 99%, or at least about 99.5% by weight of the molecules are a single compound, including a single enantiomer, a racemic mixture, or a mixture of enantiomers, as determined by standard analytical methods. As used herein, when an atom at a particular position in an isotopically enriched molecule is designated as a particular less prevalent isotope, a molecule that contains other than the designated isotope at the specified position is an impurity with respect to the isotopically enriched compound. Thus, for a deuterated compound that has an atom at a particular position designated as deuterium, a compound that contains a protium at the same position is an impurity.

The term “solvate” refers to a complex or aggregate formed by one or more molecules of a solute, e.g., a compound provided herein, and one or more molecules of a solvent, which are present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

For a divalent group described herein, no orientation is implied by the direction in which the divalent group is presented. For example, unless a particular orientation is specified, the formula —C(O)NH— represents both —C(O)NH— and —NHC(O)—.

The phrase “a diastereomer, a mixture of two or more diastereomers, a tautomer, a mixture of two or more tautomers, or an isotopic variant thereof; or a pharmaceutically acceptable salt, solvate, hydrate, or prodrug thereof” has the same meaning as the phrase “(i) a diastereomer, a mixture of two or more diastereomers, a tautomer, a mixture of two or more tautomers, or an isotopic variant of the compound referenced therein; (ii) a pharmaceutically acceptable salt, solvate, hydrate, or prodrug of the compound referenced therein; or (iii) a pharmaceutically acceptable salt, solvate, hydrate, or prodrug of a diastereomer, a mixture of two or more diastereomers, a tautomer, a mixture of two or more tautomers, or an isotopic variant of the compound referenced therein.”

### Compounds

In one embodiment, provided herein is a compound of Formula (I):

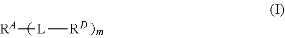

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
   R^{A} is an antibody or an antigen-binding fragment thereof;
   each L is independently a linker;
   m is an integer of 1, 2, 3, 4, 5, 6, 7, 8, 9, 10, 11, 12, 13, 14, 15, or 16; and
   each R^{D} is independently

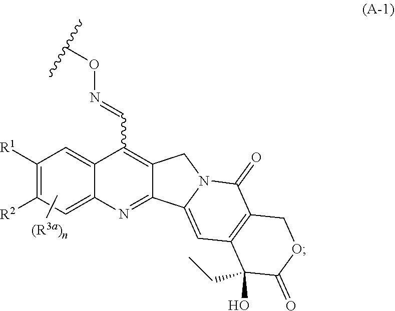

wherein:
   R¹ and R² are each independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1C}, —C(O)SR^{1a}, —C(NR^{1a})NR^{1b}R^{1c}, —C(S)R^{1a}, —C(S)OR^{1a}, —C(S)NR^{1b}R^{e}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{e}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; each R^{3a} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c} or —S(O)₂NR^{1b}R^{1c};
   each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and
   n is an integer of 0, 1, 2, 3, 4, 5, 6, 7, or 8;
wherein each alkyl, heteroalkyl, alkenyl, alkynyl, cycloalkyl, aryl, aralkyl, heteroaryl, and heterocyclyl is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e} C(O)NR^{f}R^{g}, —NR^{e}C(O)SR^{f}, —NR^{e} C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In another embodiment, provided herein is a compound of Formula (II):

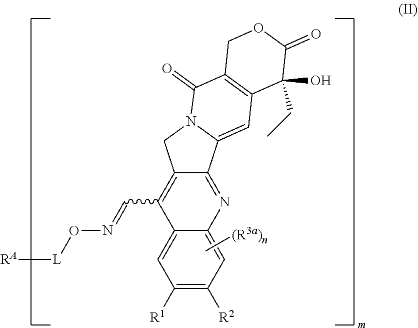

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{A}, L, m, and n are each as defined herein.

In certain embodiments, in Formula (I) or (II), each R^{3a} is independently (i) deuterium or halo; (ii) C₁₋₆ alkyl or C₁₋₆ heteroalkyl, each optionally substituted with one or more substituents Q; or (iii) —OR^{1a} or —OC(O)R^{1a}, where each R^{1a} is as defined herein. In certain embodiments, in Formula (I) or (II), each R^{3a} is independently (i) deuterium or halo; or (ii) C₁₋₆ alkyl or C₁₋₆ heteroalkyl, each optionally substituted with one or more substituents Q. In certain embodiments, in Formula (I) or (II), each R^{3a} is independently deuterium or halo. In certain embodiments, in Formula (I) or (II), each R^{3a} is independently deuterium or fluoro. In certain embodiments, in Formula (I) or (II), each R^{3a} is independently C₁₋₆ alkyl or C₁₋₆ heteroalkyl, each optionally substituted with one or more substituents Q. In certain embodiments, in Formula (I) or (II), each R^{3a} is independently deuterium, fluoro, or methyl.

In certain embodiments, in Formula (I) or (II), each n is independently an integer of 0, 1, or 2. In certain embodiments, in Formula (I) or (II), each n is an integer of 0. In certain embodiments, in Formula (I) or (II), each n is an integer of 1. In certain embodiments, in Formula (I) or (II), each n is an integer of 2.

In yet another embodiment, provided herein is a compound of Formula (III):

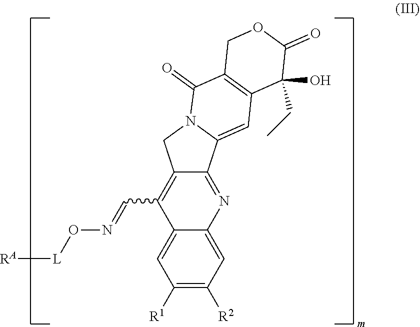

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{A}, L, and m are each as defined herein.

In yet another embodiment, provided herein is a compound of Formula (IV):

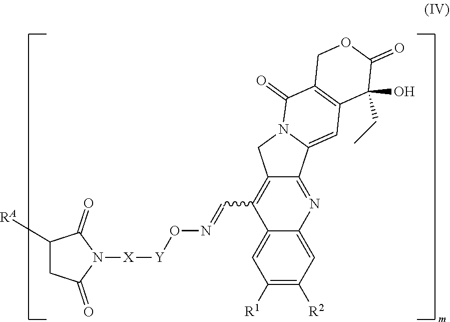

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
   each X is independently C₁₋₄₀ alkylene, C₁₋₄₀ heteroalkylene, C₂₋₄₀ alkenylene, C₂₋₄₀ heteroalkenylene, C₂₋₄₀ alkynylene, or C₂₋₄₀ heteroalkynylene, wherein one or more methylene groups are each independently and optionally replaced by a divalent group and each divalent group is independently C₃₋₁₀ cycloalkylene, C₆₋₁₄ arylene, heteroarylene, or heterocyclylene;
   each Y is independently a bond, C₁₋₆ alkylene, C₁₋₆ heteroalkylene, C₂₋₆ alkenylene, C₂₋₆ alkynylene, C₃₋₁₀ cycloalkylene, C₆₋₁₄ arylene, C₇₋₁₅ aralkylene, heteroarylene, or heterocyclylene; and
   R¹, R², R^{A}, and m are each as defined herein;
   wherein each alkylene, heteroalkylene, alkenylene, heteroalkenylene, alkynylene, heteroalkynylene, cycloalkylene, arylene, heteroarylene, and heterocyclylene is optionally substituted with one or more substituents Q.

In certain embodiments, in any one of Formulae (I) to (IV), each R¹ and R² is independently (i) hydrogen, deuterium, or halo; (ii) C₁₋₆ alkyl or C₁₋₆ heteroalkyl, each optionally substituted with one or more substituents Q; or (iii) —OR^{1a} or —OC(O)R^{1a}, where each R^{1a} is as defined herein. In certain embodiments, in any one of Formulae (I) to (IV), each R¹ and R² is independently hydrogen, deuterium, fluoro, methyl, hydroxyl, or 4-(piperidin-1-yl)piperidin-1-ylcarbonyl.

In certain embodiments, in any one of Formulae (I) to (IV), each R¹ is independently (i) hydrogen, deuterium, or halo; (ii) C₁₋₆ alkyl or C₁₋₆ heteroalkyl, each optionally substituted with one or more substituents Q; or (iii) —OR^{1a} or —OC(O)R^{1a}, where each R^{1a} is as defined herein. In certain embodiments, in any one of Formulae (I) to (IV), each R¹ is independently hydrogen or deuterium. In certain embodiments, in any one of Formulae (I) to (IV), each R¹ is independently halo. In certain embodiments, in any one of Formulae (I) to (IV), each R¹ is independently fluoro, chloro, or bromo. In certain embodiments, in any one of Formulae (I) to (IV), each R¹ is fluoro. In certain embodiments, in any one of Formulae (I) to (IV), each R¹ is independently C₁₋₆ alkyl, optionally substituted with one or more substituents Q. In certain embodiments, in any one of Formulae (I) to (IV), each R¹ is methyl. In certain embodiments, in any one of Formulae (I) to (IV), each R¹ is independently C₁₋₆ heteroalkyl, optionally substituted with one or more substituents Q. In certain embodiments, in any one of Formulae (I) to (IV), each R¹ is trifluoromethyl. In certain embodiments, in any one of Formulae (I) to (IV), each R¹ is independently —OR^{1a}, where each R^{1a} is as defined herein. In certain embodiments, in any one of Formulae (I) to (IV), each R¹ is hydroxyl. In certain embodiments, in any one of Formulae (I) to (IV), each R¹ is independently —OC(O)R^{1a}, where each R^{1a} is as defined herein. In certain embodiments, in any one of Formulae (I) to (IV), each R¹ is 4-(piperidin-1-yl)piperidin-1-ylcarbonyl. In certain embodiments, in any one of Formulae (I) to (IV), each R¹ is independently hydrogen, deuterium, fluoro, methyl, hydroxyl, or 4-(piperidin-1-yl)piperidin-1-ylcarbonyl. In certain embodiments, in any one of Formulae (I) to (IV), each R¹ is independently hydrogen, methyl, hydroxyl, or 4-(piperidin-1-yl)piperidin-1-ylcarbonyl.

In certain embodiments, in any one of Formulae (I) to (IV), each R² is independently (i) hydrogen, deuterium, or halo; (ii) C₁₋₆ alkyl or C₁₋₆ heteroalkyl, each optionally substituted with one or more substituents Q; or (iii) —OR^{1a} or —OC(O)R^{1a}, where each R^{1a} is as defined herein. In certain embodiments, in any one of Formulae (I) to (IV), each R² is independently hydrogen or deuterium. In certain embodiments, in any one of Formulae (I) to (IV), each R² is independently halo. In certain embodiments, in any one of Formulae (I) to (IV), each R² is independently fluoro, chloro, or bromo. In certain embodiments, in any one of Formulae (I) to (IV), each R² is fluoro. In certain embodiments, in any one of Formulae (I) to (IV), each R² is independently C₁₋₆ alkyl, optionally substituted with one or more substituents Q. In certain embodiments, in any one of Formulae (I) to (IV), each R² is methyl. In certain embodiments, in any one of Formulae (I) to (IV), each R² is independently C₁₋₆ heteroalkyl, optionally substituted with one or more substituents Q. In certain embodiments, in any one of Formulae (I) to (IV), each R² is trifluoromethyl. In certain embodiments, in any one of Formulae (I) to (IV), each R² is independently —OR^{1a}, where each R^{1a} is as defined herein. In certain embodiments, in any one of Formulae (I) to (IV), each R² is hydroxyl. In certain embodiments, in any one of Formulae (I) to (IV), each R² is independently —OC(O)R^{1a}, where each R^{1a} is as defined herein. In certain embodiments, in any one of Formulae (I) to (IV), each R² is 4-(piperidin-1-yl)piperidin-1-ylcarbonyl. In certain embodiments, in any one of Formulae (I) to (IV), each R² is independently hydrogen, deuterium, fluoro, methyl, hydroxyl, or 4-(piperidin-1-yl)piperidin-1-ylcarbonyl. In certain embodiments, in any one of Formulae (I) to (IV), each R² is independently hydrogen, fluoro, or methyl.

In yet another embodiment, provided herein is a compound of Formula (V):

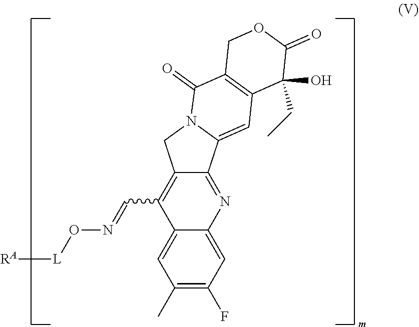

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R^{A}, L, and m are each as defined herein.

In still another embodiment, provided herein is a compound of Formula (VI):

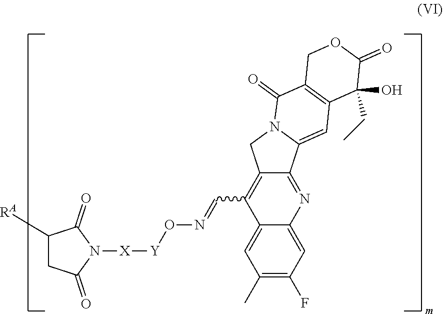

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R^{A}, X, Y, and m are each as defined herein.

In one embodiment, R^{A} is a full length or intact monoclonal antibody. In another embodiment, R^{A} is a monoclonal antibody or an antigen-binding fragment thereof. In yet another embodiment, R^{A} is a single domain antibody or an antigen-binding fragment thereof. In yet another embodiment, R^{A} is a single domain antibody or an antigen-binding fragment thereof. In yet another embodiment, R^{A} is a V_{H}H antibody. In still another embodiment, R^{A} is a V_{NAR} antibody.

In one embodiment, R^{A} is a human, humanized, or chimeric antibody, or an antigen-binding fragment thereof. In another embodiment, R^{A} is a human antibody or an antigen-binding fragment thereof. In yet another embodiment, R^{A} is a humanized antibody or an antigen-binding fragment thereof. In still another embodiment, R^{A} is a chimeric antibody or an antigen-binding fragment thereof.

In one embodiment, R^{A} is an IgA, IgD, IgE, IgG, or IgM antibody, or an antigen-binding fragment thereof. In another embodiment, R^{A} is an IgA antibody or an antigen-binding fragment thereof. In yet another embodiment, R^{A} is an IgD antibody or an antigen-binding fragment thereof. In yet another embodiment, R^{A} is an IgE antibody or an antigen-binding fragment thereof. In yet another embodiment, R^{A} is an IgG antibody or an antigen-binding fragment thereof. In still another embodiment, R^{A} is an IgM antibody or an antigen-binding fragment thereof.

In one embodiment, R^{A} is an IgA1, IgA2, IgG1, IgG2, IgG3, or IgG4 antibody, or an antigen-binding fragment thereof. In another embodiment, R^{A} is an IgA1 or IgA2, or an antigen-binding fragment thereof. In yet another embodiment, R^{A} is an IgA1 or an antigen-binding fragment thereof. In yet another embodiment, R^{A} is an IgA2 or an antigen-binding fragment thereof. In yet another embodiment, R^{A} is an IgG1, IgG2, IgG3, or IgG4 antibody, or an antigen-binding fragment thereof. In yet another embodiment, R^{A} is an IgG1 antibody or an antigen-binding fragment thereof. In yet another embodiment, R^{A} is an IgG2 antibody or an antigen-binding fragment thereof. In yet another embodiment, R^{A} is an IgG3 antibody or an antigen-binding fragment thereof. In still another embodiment, R^{A} is an IgG4 antibody or an antigen-binding fragment thereof.

In one embodiment, R^{A} is a single-chain variable fragment (scFv), Fab, Fab′, F(ab)₂, F(ab′)₂, Fv, diabody, triabody, tetrabody, or minibody of an antibody provided herein. In another embodiment, R^{A} is an scFv of an antibody provided herein. In yet another embodiment, R^{A} is a Fab of an antibody provided herein. In yet another embodiment, R^{A} is a Fab′ of an antibody provided herein. In yet another embodiment, R^{A} is a F(ab)₂ of an antibody provided herein. In yet another embodiment, R^{A} is a F(ab′)₂ of an antibody provided herein. In yet another embodiment, R^{A} is a Fv of an antibody provided herein. In yet another embodiment, R^{A} is a diabody of an antibody provided herein. In yet another embodiment, R^{A} is a triabody of an antibody provided herein. In yet another embodiment, R^{A} is a tetrabody of an antibody provided herein. In still another embodiment, R^{A} is a minibody of an antibody provided herein.

In one embodiment, R^{A} is an afucosylated antibody or an antigen-binding fragment thereof.

In one embodiment, R^{A} is a recombinant antibody or an antigen-binding fragment thereof. In another embodiment, R^{A} is a purified antibody or an antigen-binding fragment thereof. In yet another embodiment, R^{A} is an isolated antibody or an antigen-binding fragment thereof.

In certain embodiments, R^{A} is an antibody or an antigen-binding fragment thereof, wherein the antibody specifically binds to AXL, B7 homolog 3 (B7H3), B-cell maturation antigen (BCMA), cadherin 3, cadherin 6, CanAg, carbonic anhydrase VI (CA6), C4-4A, carcinoembryonic antigen-related cell adhesion molecules (CEACAMs), CD19, CD20, CD22, CD25, CD30, CD33, CD37, CD44, CD56, CD70, CD74, CD79b, CD123, CD138, CD142, CD352, cripto 1 growth factor, delta-like 3 (DLL3), ectonucleotide pyrophosphatase/phosphor-diesterase family member 3 (ENPP3), endothelin receptor type B (ETBR), EPH receptor A2 (EPHA2), ephrin A4, epidermal growth factor receptor (EGFR), FMS-like tyrosine kinase 3 (FLT3), fibroblast growth factor receptor 2 (FGFR2), fibroblast growth factor receptor 3 (FGFR3), folate receptor 1 (FOLR1 or FRa), guanylate cyclase 2C (GCC), hepatocyte growth factor receptor (HGFR), HER2, HER3, integrin aV, KIT, Lewis Y antigen, LIV-1, lymphocyte antigen 6E (LY6E), lysosomal-associated membrane protein 1 (LAMP-1), mesothelin (MSLN), mucin 1 (MUC1), mucin-16 (MUC16), nectin-4, notch receptor 3 (NOTCH3), osteoactivin, prostate-specific membrane antigen (PSMA), six transmembrane epithelial antigen of the prostate 1 (STEAPI), SLAM family member 7 (SLAMF7), SLITRK6, ST2, sodium-dependent phosphate transport protein 2B (SLC34A2), T-cell immunoglobulin and mucin domain 1 (TIM-1), transferrin receptor protein 1, tumor-associated calcium signal transducer 2 (TROP2), or tyrosine-protein kinase-like 7 (PTK7).

In certain embodiments, R^{A} is an anti-B7H3 antibody, anti-CD20 antibody, anti-FOLR1 antibody, anti-HER2 antibody, anti-MSLN antibody, anti-TROP2 antibody, or an antigen-binding fragment thereof. In certain embodiments, R^{A} is an anti-B7H3 antibody or an antigen-binding fragment thereof. In certain embodiments, R^{A} is an anti-CD20 antibody or an antigen-binding fragment thereof. In certain embodiments, R^{A} is an anti-FOLR1 antibody or an antigen-binding fragment thereof. In certain embodiments, R^{A} is an anti-HER2 antibody or an antigen-binding fragment thereof. In certain embodiments, R^{A} is an anti-MSLN antibody or an antigen-binding fragment thereof. In certain embodiments, R^{A} is an anti-TROP2 antibody or an antigen-binding fragment thereof.

In one embodiment, R^{A} is an anti-B7H3 single domain antibody or an antigen-binding fragment thereof, wherein the antibody comprises (i) a chain complementarity determining region 1 (CDR1) of SEQ ID NO: 1; (ii) a chain complementarity determining region 2 (CDR2) of SEQ ID NO: 2; and (iii) a chain complementarity determining region 3 (CDR3) of SEQ ID NO: 3. In certain embodiments, the CDRs provided herein are defined according to the IMGT or Kabat numbering system. In certain embodiments, the CDRs provided herein are defined according to the IMGT numbering system. In certain embodiments, the CDRs provided herein are defined according to the Kabat numbering system. In another embodiment, R^{A} is an anti-B7H3 single domain antibody or an antigen-binding fragment thereof, wherein the antibody comprises a variable region of SEQ ID NO: 4. In yet another embodiment, R^{A} is an anti-B7H3 single domain antibody or an antigen-binding fragment thereof, wherein the antibody comprises an amino acid sequence of SEQ ID NO: 5.

In one embodiment, R^{A} is an anti-B7H3 single domain antibody or an antigen-binding fragment thereof, wherein the antibody comprises (i) a CDR1 of SEQ ID NO: 6; (ii) a CDR2 of SEQ ID NO: 7; and (iii) a CDR3 of SEQ ID NO: 8. In certain embodiments, the CDRs provided herein are defined according to the IMGT or Kabat numbering system. In certain embodiments, the CDRs provided herein are defined according to the IMGT numbering system. In certain embodiments, the CDRs provided herein are defined according to the Kabat numbering system. In another embodiment, R^{A} is an anti-B7H3 single domain antibody or an antigen-binding fragment thereof, wherein the antibody comprises a variable region of SEQ ID NO: 9. In yet another embodiment, R^{A} is an anti-B7H3 single domain antibody or an antigen-binding fragment thereof, wherein the antibody comprises an amino acid sequence of SEQ ID NO: 10.

In one embodiment, R^{A} is an anti-CD20 antibody or an antigen-binding fragment thereof, wherein the antibody comprises (i) a light chain complementarity determining region 1 (CDRL1) of SEQ ID NO: 11; (ii) a light chain complementarity determining region 2 (CDRL2) of SEQ ID NO: 12; (iii) a light chain complementarity determining region 3 (CDRL3) of SEQ ID NO: 13; (iv) a heavy chain complementarity determining region 1 (CDRH1) of SEQ ID NO: 14; (v) a heavy chain complementarity determining region 2 (CDRH2) of SEQ ID NO: 15; and (vi) a heavy chain complementarity determining region 3 (CDRH3) of SEQ ID NO: 16. In certain embodiments, the CDRs provided herein are defined according to the IMGT or Kabat numbering system. In certain embodiments, the CDRs provided herein are defined according to the IMGT numbering system. In certain embodiments, the CDRs provided herein are defined according to the Kabat numbering system. In another embodiment, R^{A} is an anti-CD20 antibody or an antigen-binding fragment thereof, wherein the antibody comprises (i) a light chain variable region of SEQ ID NO: 17; and (ii) a heavy chain variable region of SEQ ID NO: 18. In yet another embodiment, R^{A} is an anti-CD20 antibody or an antigen-binding fragment thereof, wherein the antibody comprises (i) a light chain of SEQ ID NO: 19; and (ii) a heavy chain of SEQ ID NO: 20.

In one embodiment, R^{A} is an anti-FOLR1 antibody or an antigen-binding fragment thereof, wherein the antibody comprises (i) a CDRL1 of SEQ ID NO: 21; (ii) a CDRL2 of SEQ ID NO: 22; (iii) a CDRL3 of SEQ ID NO: 23; (iv) a CDRH1 of SEQ ID NO: 24; (v) a CDRH2 of SEQ ID NO: 25; and (vi) a CDRH3 of SEQ ID NO: 26. In certain embodiments, the CDRs provided herein are defined according to the IMGT or Kabat numbering system. In certain embodiments, the CDRs provided herein are defined according to the IMGT numbering system. In certain embodiments, the CDRs provided herein are defined according to the Kabat numbering system. In another embodiment, R^{A} is an anti-FOLR1 antibody or an antigen-binding fragment thereof, wherein the antibody comprises (i) a light chain variable region of SEQ ID NO: 27; and (ii) a heavy chain variable region of SEQ ID NO: 28. In yet another embodiment, R^{A} is an anti-FOLR1 antibody or an antigen-binding fragment thereof, wherein the antibody comprises (i) a light chain of SEQ ID NO: 29; and (ii) a heavy chain of SEQ ID NO: 30.

In one embodiment, R^{A} is an anti-HER2 antibody or an antigen-binding fragment thereof, wherein the antibody comprises (i) a CDRL1 of SEQ ID NO: 31; (ii) a CDRL2 of SEQ ID NO: 32; (iii) a CDRL3 of SEQ ID NO: 33; (iv) a CDRH1 of SEQ ID NO: 34; (v) a CDRH2 of SEQ ID NO: 35; and (vi) a CDRH3 of SEQ ID NO: 36. In certain embodiments, the CDRs provided herein are defined according to the IMGT or Kabat numbering system. In certain embodiments, the CDRs provided herein are defined according to the IMGT numbering system. In certain embodiments, the CDRs provided herein are defined according to the Kabat numbering system. In another embodiment, R^{A} is an anti-HER2 antibody or an antigen-binding fragment thereof, wherein the antibody comprises (i) a light chain variable region of SEQ ID NO: 37; and (ii) a heavy chain variable region of SEQ ID NO: 38. In yet another embodiment, R^{A} is an anti-HER2 antibody or an antigen-binding fragment thereof, wherein the antibody comprises (i) a light chain of SEQ ID NO: 39; and (ii) a heavy chain of SEQ ID NO: 40.

In one embodiment, R^{A} is an anti-MSLN single domain antibody or an antigen-binding fragment thereof, wherein the antibody comprises (i) a CDR1 of SEQ ID NO: 41; (ii) a CDR2 of SEQ ID NO: 42; and (iii) a CDR3 of SEQ ID NO: 43. In certain embodiments, the CDRs provided herein are defined according to the IMGT or Kabat numbering system. In certain embodiments, the CDRs provided herein are defined according to the IMGT numbering system. In certain embodiments, the CDRs provided herein are defined according to the Kabat numbering system. In another embodiment, R^{A} is an anti-MSLN single domain antibody or an antigen-binding fragment thereof, wherein the antibody comprises a variable region of SEQ ID NO: 44. In yet another embodiment, R^{A} is an anti-MSLN single domain antibody or an antigen-binding fragment thereof, wherein the antibody comprises an amino acid sequence of SEQ ID NO: 45.

In one embodiment, R^{A} is an anti-MSLN single domain antibody or an antigen-binding fragment thereof, wherein the antibody comprises (i) a CDR1 of SEQ ID NO: 46; (ii) a CDR2 of SEQ ID NO: 47; and (iii) a CDR3 of GRY. In certain embodiments, the CDRs provided herein are defined according to the IMGT or Kabat numbering system. In certain embodiments, the CDRs provided herein are defined according to the IMGT numbering system. In certain embodiments, the CDRs provided herein are defined according to the Kabat numbering system. In another embodiment, R^{A} is an anti-MSLN single domain antibody or an antigen-binding fragment thereof, wherein the antibody comprises a variable region of SEQ ID NO: 48. In yet another embodiment, R^{A} is an anti-MSLN single domain antibody or an antigen-binding fragment thereof, wherein the antibody comprises an amino acid sequence of SEQ ID NO: 49.

Additional anti-MSLN single domain antibodies and antigen-binding fragments thereof include those disclosed in WO 2019/246003 A1, the disclosure of which is incorporated herein by reference in its entirety.

In one embodiment, R^{A} is an anti-TROP2 antibody or an antigen-binding fragment thereof, wherein the antibody comprises (i) a CDRL1 of SEQ ID NO: 50; (ii) a CDRL2 of SEQ ID NO: 51; (iii) a CDRL3 of SEQ ID NO: 52; (iv) a CDRH1 of SEQ ID NO: 53; (v) a CDRH2 of SEQ ID NO: 54; and (vi) a CDRH3 of SEQ ID NO: 55. In certain embodiments, the CDRs provided herein are defined according to the IMGT or Kabat numbering system. In certain embodiments, the CDRs provided herein are defined according to the IMGT numbering system. In certain embodiments, the CDRs provided herein are defined according to the Kabat numbering system. In another embodiment, R^{A} is an anti-TROP2 antibody or an antigen-binding fragment thereof, wherein the antibody comprises (i) a light chain variable region of SEQ ID NO: 56; and (ii) a heavy chain variable region of SEQ ID NO: 57. In yet another embodiment, R^{A} is an anti-TROP2 antibody or an antigen-binding fragment thereof, wherein the antibody comprises (i) a light chain of SEQ ID NO: 58; and (ii) a heavy chain of SEQ ID NO: 59.

In one embodiment, L is a cleavable linker. In another embodiment, L is a non-cleavable linker.

In certain embodiments, L is a cleavable linker that is sensitive to an acidic pH. In certain embodiments, L is a cleavable linker comprising a reducible disulfide. In certain embodiments, L is a linker cleavable by glutathione. In certain embodiments, L is a linker cleavable by an enzyme. In certain embodiments, L is a linker cleavable by a protease. In certain embodiments, L is a linker cleavable by a lysosomal protease. In certain embodiments, L is a linker cleavable by cathepsin B. In certain embodiments, L is a linker cleavable by a glycosidase. In certain embodiments, L is a linker cleavable by a β-glycosidase. In certain embodiments, L is a linker cleavable by a galactosidase. In certain embodiments, L is a linker cleavable by a β-galactosidase. In certain embodiments, L is a linker cleavable by a glucuronidase. In certain embodiments, L is a linker cleavable by a β-glucuronidase. In certain embodiments, L is a linker cleavable by a phosphatase. Exemplary linkers suitable for a compound provided herein include, but are not limited to, those disclosed in Beck et al., Nat. Rev. Drug Discov. 2017, 16, 317-37; Bargh et al., Chem. Soc. Rev. 2019, 48, 4361-74; the disclosure of each of which is incorporated herein by reference in its entirety.

In certain embodiments, L is C₁₋₅₀ alkylene, C₁₋₅₀ heteroalkylene, C₂₋₅₀ alkenylene, C₂₋₅₀ heteroalkenylene, C₂₋₅₀ alkynylene, C₂₋₅₀ heteroalkynylene, C₃₋₁₀ cycloalkylene, C₆₋₁₄ arylene, heteroarylene, or heterocyclylene, each of which is optionally substituted with one or more substituents Q. In certain embodiments, L is C₁₋₅₀ alkylene, C₁₋₅₀ heteroalkylene, C₂₋₅₀ alkenylene, C₂₋₅₀ heteroalkenylene, C₂₋₅₀ alkynylene, or C₂₋₅₀ heteroalkynylene, each of which is optionally substituted with one or more substituents Q. In certain embodiments, L is C₁₋₅₀ alkylene or C₁₋₅₀ heteroalkylene, each of which is optionally substituted with one or more substituents Q.

In certain embodiments, L is C₁₋₅₀ alkylene, optionally substituted with one or substituents Q. In certain embodiments, L is C₁₋₄₀ alkylene, optionally substituted with one or more substituents Q. In certain embodiments, L is C₁₋₃₀ alkylene, optionally substituted with one or more substituents Q. In certain embodiments, L is C₁₋₂₀ alkylene, optionally substituted with one or more substituents Q. In certain embodiments, L is C₂₋₁₆ alkylene, optionally substituted with one or more substituents Q. In certain embodiments, L is C₂₋₁₂ alkylene, optionally substituted with one or more substituents Q. In certain embodiments, L is C₂₋₆ alkylene, optionally substituted with one or more substituents Q. In certain embodiments, L is C₁₋₂₀ alkylene, optionally substituted with one or two oxo. In certain embodiments, L is C₂₋₁₆ alkylene, optionally substituted with one or two oxo. In certain embodiments, L is C₂₋₁₂ alkylene, optionally substituted with one or two oxo. In certain embodiments, L is C₂₋₆ alkylene, optionally substituted with one or two oxo. In certain embodiments, L is —(CH₂)₅—, optionally substituted with one or two oxo; wherein s is an integer of 1, 2, 3, 4, 5, 6, 7, 8, 9, 10, 11, 12, 13, 14, 15, or 16. In certain embodiments, L is —(CH₂)₅—, optionally substituted with one or two oxo; wherein s is an integer of 2, 3, 4, 5, 6, 7, 8, 9, 10, 11, or 12. In certain embodiments, L is —(CH₂)₅—, optionally substituted with one or two oxo; wherein s is an integer of 2, 3, 4, 5, or 6.

In certain embodiments, L is C₁₋₅₀ heteroalkylene, optionally substituted with one or more substituents Q. In certain embodiments, L is C₁₋₄₀ heteroalkylene, optionally substituted with one or more substituents Q. In certain embodiments, L is C₁₋₃₀ heteroalkylene, optionally substituted with one or more substituents Q. In certain embodiments, L is C₁₋₂₀ heteroalkylene, optionally substituted with one or more substituents Q. In certain embodiments, L is C₂₋₁₆ heteroalkylene, optionally substituted with one or more substituents Q. In certain embodiments, L is C₂₋₁₂ heteroalkylene, optionally substituted with one or more substituents Q. In certain embodiments, L is C₂₋₆ heteroalkylene, optionally substituted with one or more substituents Q. In certain embodiments, L is C₁₋₂₀ heteroalkylene, optionally substituted with one, two, or three oxo. In certain embodiments, L is C₂₋₁₆ heteroalkylene, optionally substituted with one, two, or three oxo. In certain embodiments, L is C₂₋₁₂ heteroalkylene, optionally substituted with one, two, or three oxo. In certain embodiments, L is C₂₋₆ heteroalkylene, optionally substituted with one, two, or three oxo.

In certain embodiments, L is C₂₋₅₀ heteroalkylene comprising an ethyleneoxy (—CH₂CH₂O—) group, optionally substituted with one or more substituents Q. In certain embodiments, L is C₂₋₄₀ heteroalkylene comprising an ethyleneoxy group, optionally substituted with one or more substituents Q. In certain embodiments, L is C₂₋₃₀ heteroalkylene comprising an ethyleneoxy group, optionally substituted with one or more substituents Q. In certain embodiments, L is C₂₋₂₀ heteroalkylene comprising an ethyleneoxy group, optionally substituted with one or more substituents Q. In certain embodiments, L is C₂₋₁₄ heteroalkylene comprising an ethyleneoxy group, optionally substituted with one or more substituents Q. In certain embodiments, L is C₂₋₁₀ heteroalkylene comprising an ethyleneoxy group, optionally substituted with one or more substituents Q. In certain embodiments, L is C₂₋₆ heteroalkylene comprising an ethyleneoxy group, optionally substituted with one or more substituents Q.

In certain embodiments, L is C₃₋₅₀ heteroalkylene comprising a propyleneoxy (—CH₂CH₂CH₂O—) group, optionally substituted with one or more substituents Q. In certain embodiments, L is C₃₋₄₀ heteroalkylene comprising a propyleneoxy group, optionally substituted with one or more substituents Q. In certain embodiments, L is C₃₋₃₀ heteroalkylene comprising a propyleneoxy group, optionally substituted with one or more substituents Q. In certain embodiments, L is C₃₋₂₀ heteroalkylene comprising a propyleneoxy group, optionally substituted with one or more substituents Q. In certain embodiments, L is C₃₋₁₄ heteroalkylene comprising a propyleneoxy group, optionally substituted with one or more substituents Q. In certain embodiments, L is C₃₋₁₀ heteroalkylene comprising a propyleneoxy group, optionally substituted with one or more substituents Q.

In certain embodiments, L is C₁₋₅₀ alkylene, C₁₋₅₀ heteroalkylene, C₂₋₅₀ alkenylene, C₂₋₅₀ heteroalkenylene, C₂₋₅₀ alkynylene, or C₂₋₅₀ heteroalkynylene, wherein one or more methylene groups are each independently replaced by a divalent group; wherein each divalent group is independently C₃₋₁₀ cycloalkylene, C₆₋₁₄ arylene, heteroarylene, or heterocyclylene; and wherein the alkylene, heteroalkylene, alkenylene, heteroalkenylene, alkynylene, heteroalkynylene, cycloalkylene, arylene, heteroarylene, and heterocyclylene are each optionally substituted with one or more substituents Q. In certain embodiments, L is C₁₋₅₀ alkylene or C₁₋₅₀ heteroalkylene, wherein one or more methylene groups are each independently replaced by a divalent group; wherein each divalent group is independently C₃₋₁₀ cycloalkylene, C₆₋₁₄ arylene, heteroarylene, or heterocyclylene; and wherein the alkylene, heteroalkylene, cycloalkylene, arylene, heteroarylene, and heterocyclylene are each optionally substituted with one or more substituents Q.

In certain embodiments, L is C₁₋₅₀ alkylene, wherein one or more methylene groups are each independently replaced by a divalent group; wherein each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q. In certain embodiments, L is C₁₋₄₀ alkylene, wherein one or more methylene groups are each independently replaced by a divalent group; wherein each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q. In certain embodiments, L is C₁₋₃₀ alkylene, wherein one or more methylene groups are each independently replaced by a divalent group; wherein each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q. In certain embodiments, L is C₁₋₂₀ alkylene, wherein one or more methylene groups are each independently replaced by a divalent group; wherein each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q.

In certain embodiments, L is C₁₋₅₀ alkylene, wherein one, two, three, or four methylene groups are each independently replaced by a divalent group; wherein each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q. In certain embodiments, L is C₁₋₄₀ alkylene, wherein one, two, three, or four methylene groups are each independently replaced by a divalent group; wherein each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q. In certain embodiments, L is C₁₋₃₀ alkylene, wherein one, two, three, or four methylene groups are each independently replaced by a divalent group; wherein each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q. In certain embodiments, L is C₁₋₂₀ alkylene, wherein one, two, three, or four methylene groups are each independently replaced by a divalent group; wherein each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q. In certain embodiments, L is C₁₋₂₀ alkylene, wherein one, two, three, or four methylene groups are each independently replaced by a divalent group; wherein each divalent group is independently cyclohexanediyl, phendiyl, triazoldiyl, or 2,5-dioxopyrrolidindiyl. In certain embodiments, L is C₁₋₂₀ alkylene, wherein one, two, three, or four methylene groups are each independently replaced by a divalent group; wherein each divalent group is independently cyclohexane-1,4-diyl, phen-1,3-diyl, phen-1,4-diyl, 1,2,3-triazol-1,4-diyl, or 2,5-dioxopyrrolidin-1,3-diyl.

In certain embodiments, L is C₁₋₂₀ alkylene, wherein one or two methylene groups are each independently replaced by a divalent group; wherein each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q. In certain embodiments, L is C₁₋₂₀ alkylene, wherein one or two methylene groups are each independently replaced by a divalent group; wherein each divalent group is independently cyclohexanediyl, phendiyl, triazoldiyl, or 2,5-dioxopyrrolidindiyl. In certain embodiments, L is C₁₋₂₀ alkylene, wherein one or two methylene groups are each independently replaced by a divalent group; wherein each divalent group is independently cyclohexane-1,4-diyl, phen-1,3-diyl, phen-1,4-diyl, 1,2,3-triazol-1,4-diyl, or 2,5-dioxopyrrolidin-1,3-diyl.

In certain embodiments, L is C₁₋₂₀ alkylene, wherein a methylene group is replaced by a divalent group; wherein the divalent group is C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q. In certain embodiments, L is C₁₋₂₀ alkylene, wherein a methylene group is replaced by a divalent group; wherein the divalent group is cyclohexanediyl, phendiyl, triazoldiyl, or 2,5-dioxopyrrolidindiyl. In certain embodiments, L is C₁₋₂₀ alkylene, wherein a methylene group is replaced by a divalent group; wherein each divalent group is independently cyclohexane-1,4-diyl, phen-1,3-diyl, phen-1,4-diyl, 1,2,3-triazol-1,4-diyl, or 2,5-dioxopyrrolidin-1,3-diyl.

In certain embodiments, L is C₁₋₅₀ heteroalkylene, wherein one or more methylene groups are each independently replaced by a divalent group; wherein each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q. In certain embodiments, L is C₁₋₄₀ heteroalkylene, wherein one or more methylene groups are each independently replaced by a divalent group; wherein each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q. In certain embodiments, L is C₁₋₃₀ heteroalkylene, wherein one or more methylene groups are each independently replaced by a divalent group; wherein each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q. In certain embodiments, L is C₁₋₂₀ heteroalkylene, wherein one or more methylene groups are each independently replaced by a divalent group; wherein each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q.

In certain embodiments, L is C₁₋₅₀ heteroalkylene, wherein one, two, three, or four methylene groups are each independently replaced by a divalent group; wherein each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q. In certain embodiments, L is C₁₋₄₀ heteroalkylene, wherein one, two, three, or four methylene groups are each independently replaced by a divalent group; wherein each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q. In certain embodiments, L is C₁₋₃₀ heteroalkylene, wherein one, two, three, or four methylene groups are each independently replaced by a divalent group; wherein each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q. In certain embodiments, L is C₁₋₂₀ heteroalkylene, wherein one, two, three, or four methylene groups are each independently replaced by a divalent group; wherein each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q. In certain embodiments, L is C₁₋₂₀ heteroalkylene, wherein one, two, three, or four methylene groups are each independently replaced by a divalent group; wherein each divalent group is independently cyclohexanediyl, phendiyl, triazoldiyl, or 2,5-dioxopyrrolidindiyl. In certain embodiments, L is C₁₋₂₀ heteroalkylene, wherein one, two, three, or four methylene groups are each independently replaced by a divalent group; wherein each divalent group is independently cyclohexane-1,4-diyl, phen-1,3-diyl, phen-1,4-diyl, 1,2,3-triazol-1,4-diyl, or 2,5-dioxopyrrolidin-1,3-diyl.

In certain embodiments, L is C₁₋₂₀ heteroalkylene, wherein one or two methylene groups are each independently replaced by a divalent group; wherein each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q. In certain embodiments, L is C₁₋₂₀ heteroalkylene, wherein one or two methylene groups are each independently replaced by a divalent group; wherein each divalent group is independently cyclohexanediyl, phendiyl, triazoldiyl, or 2,5-dioxopyrrolidindiyl. In certain embodiments, L is C₁₋₂₀ heteroalkylene, wherein one or two methylene groups are each independently replaced by a divalent group; wherein each divalent group is independently cyclohexane-1,4-diyl, phen-1,3-diyl, phen-1,4-diyl, 1,2,3-triazol-1,4-diyl, or 2,5-dioxopyrrolidin-1,3-diyl.

In certain embodiments, L is C₁₋₂₀ heteroalkylene, wherein a methylene group is replaced by a divalent group; wherein the divalent group is C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q. In certain embodiments, L is C₁₋₂₀ heteroalkylene, wherein a methylene group is replaced by a divalent group; wherein the divalent group is cyclohexanediyl, phendiyl, triazoldiyl, or 2,5-dioxopyrrolidindiyl. In certain embodiments, L is C₁₋₂₀ heteroalkylene, wherein a methylene group is replaced by a divalent group; wherein each divalent group is independently cyclohexane-1,4-diyl, phen-1,3-diyl, phen-1,4-diyl, 1,2,3-triazol-1,4-diyl, or 2,5-dioxopyrrolidin-1,3-diyl.

In certain embodiments, L is C₁₋₄₀ alkylene-C₃₋₁₀ cycloalkylene, C₁₋₄₀ heteroalkylene-C₃₋₁₀ cycloalkylene, C₁₋₄₀ alkylene-C₆₋₁₄ arylene, C₁₋₄₀ heteroalkylene-C₆₋₁₄ arylene, C₁₋₄₀ alkylene-heteroarylene, C₁₋₄₀ heteroalkylene-heteroarylene, C₁₋₄₀ alkylene-heterocyclylene, C₁₋₄₀ heteroalkylene-heterocyclylene, or heteroarylene-heterocyclylene, where each alkylene, heteroalkylene, cycloalkylene, arylene, heteroarylene, and heterocyclylene is optionally substituted with one or more substituents Q. In certain embodiments, L is C₁₋₄₀ alkylene-C₃₋₁₀ cycloalkylene, where the alkylene and cycloalkylene are each optionally substituted with one or more substituents Q. In certain embodiments, L is C₁₋₄₀ heteroalkylene-C₃₋₁₀ cycloalkylene, where the heteroalkylene and cycloalkylene are each optionally substituted with one or more substituents Q. In certain embodiments, L is C₁₋₄₀ alkylene-C₆₋₁₄ arylene, where the alkylene and arylene are each optionally substituted with one or more substituents Q. In certain embodiments, L is C₁₋₄₀ heteroalkylene-C₆₋₁₄ arylene, where the heteroalkylene and arylene are each optionally substituted with one or more substituents Q. In certain embodiments, L is C₁₋₄₀ alkylene-heteroarylene, where the alkylene and heteroarylene are each optionally substituted with one or more substituents Q. In certain embodiments, L is C₁₋₄₀ heteroalkylene-heteroarylene, where the heteroalkylene and heteroarylene are each optionally substituted with one or more substituents Q. In certain embodiments, L is C₁₋₄₀ alkylene-heterocyclylene, where the alkylene and heterocyclylene are each optionally substituted with one or more substituents Q. In certain embodiments, L is C₁₋₄₀ heteroalkylene-heterocyclylene, where the heteroalkylene and heterocyclylene are each optionally substituted with one or more substituents Q. In certain embodiments, L is C₁₋₄₀ heteroarylene-heterocyclylene, where the heteroarylene and heterocyclylene are each optionally substituted with one or more substituents Q.

In certain embodiments, L is: 0

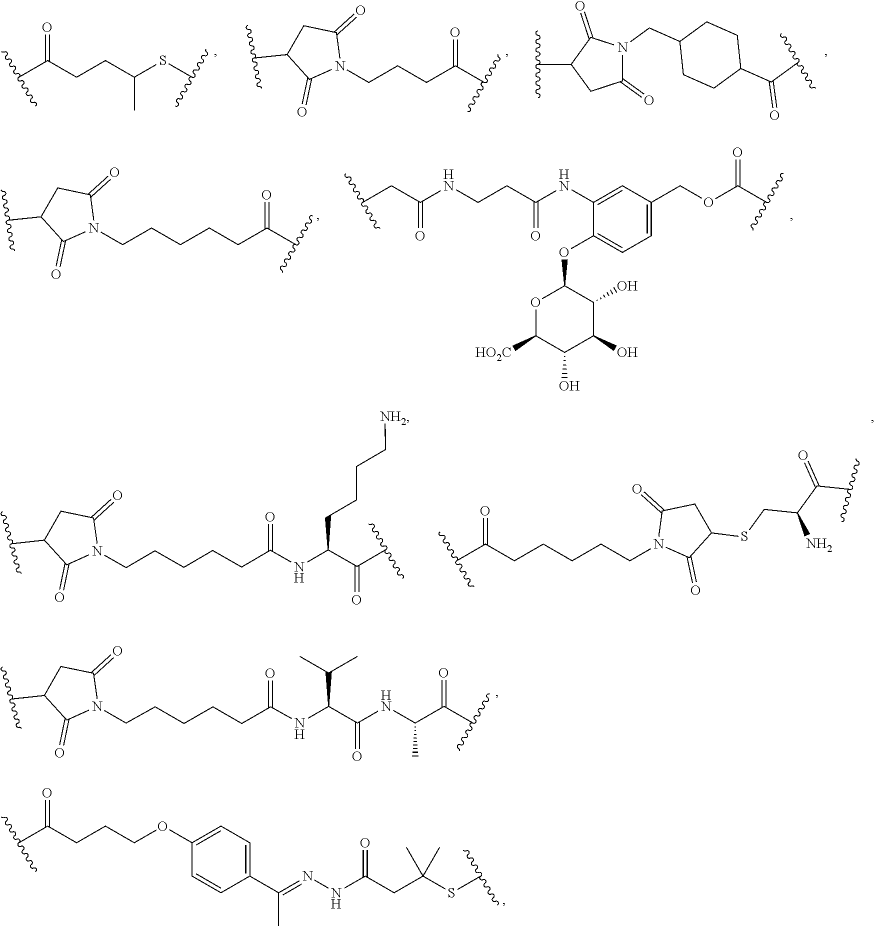

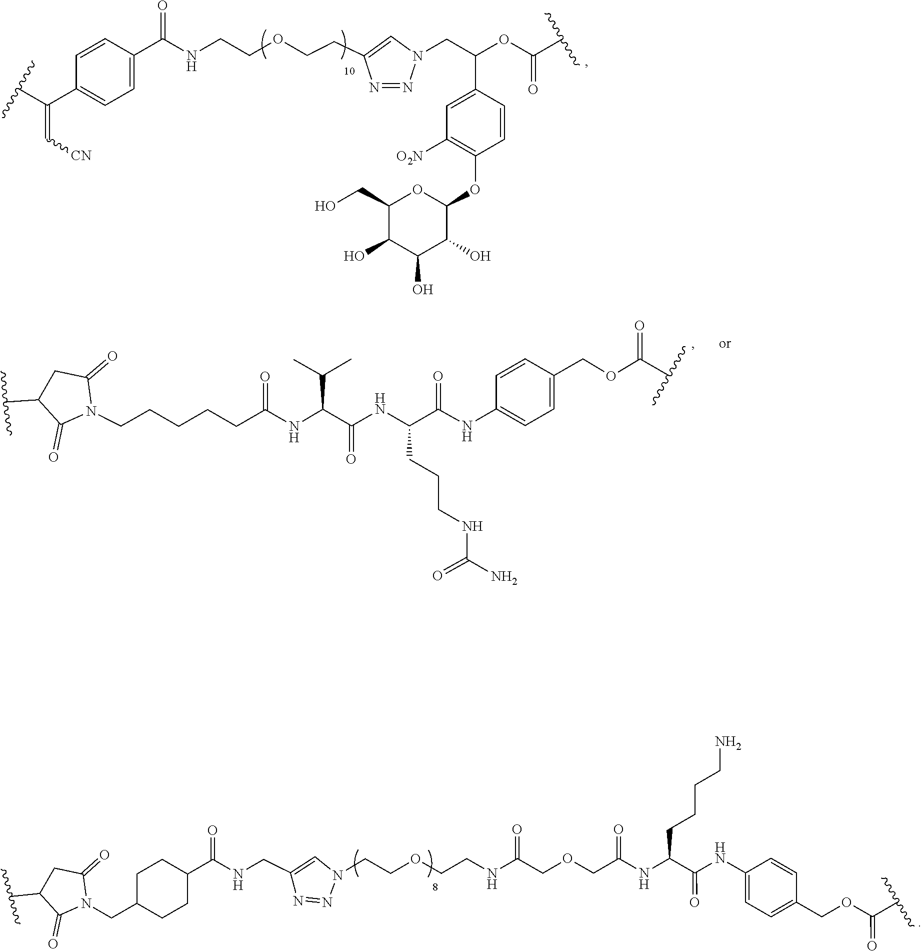

In certain embodiments, L is:

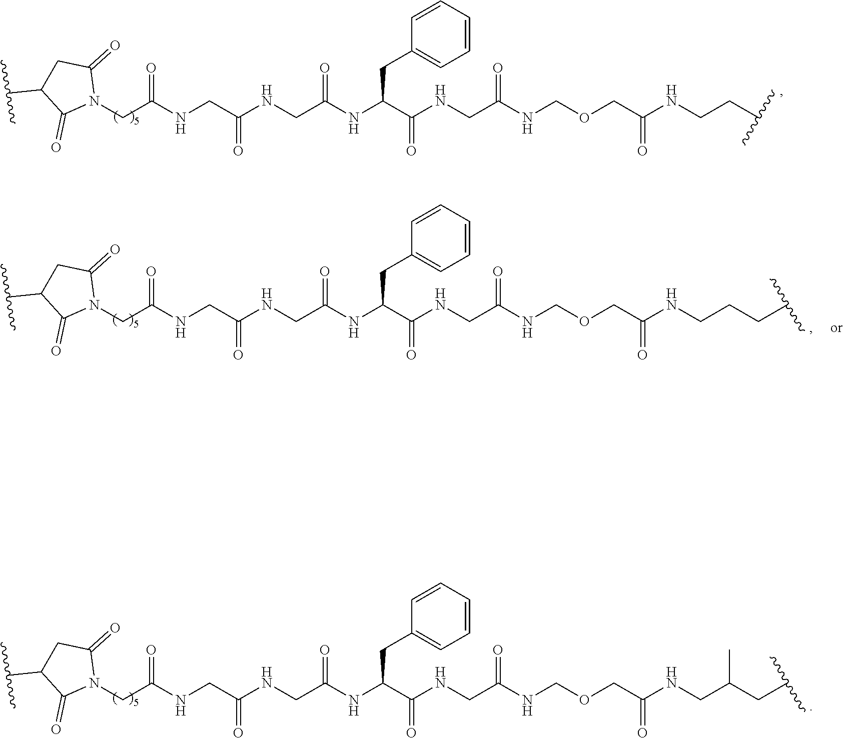

In certain embodiments, R^{D} is:

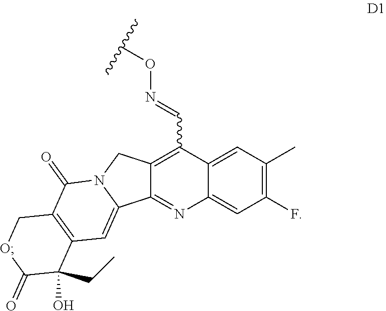 [SMILES: *ON=Cc1c2c(nc3cc(F)c(C)cc13)-c1cc3c(c(=O)n1C2)COC(=O)[C@]3(O)CC]

In one embodiment, provided herein is a compound of Formula (IA):

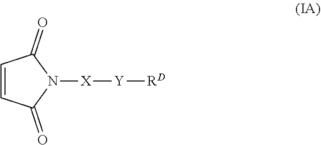

or a diastereomer, a mixture of two or more diastereomers, a tautomer, a mixture of two or more tautomers, or an isotopic variant thereof; or a pharmaceutically acceptable salt, solvate, or hydrate thereof; wherein:
   X is C₁₋₄₀ alkylene, C₁₋₄₀ heteroalkylene, C₂₋₄₀ alkenylene, C₂₋₄₀ heteroalkenylene, C₂₋₄₀ alkynylene, or C₂₋₄₀ heteroalkynylene, wherein one or more methylene groups are each independently and optionally replaced by a divalent group and each divalent group is independently C₃₋₁₀ cycloalkylene, C₆₋₁₄ arylene, heteroarylene, or heterocyclylene;
   Y is independently a bond, C₁₋₆ alkylene, C₁₋₆ heteroalkylene, C₂₋₆ alkenylene, C₂₋₆ alkynylene, C₃₋₁₀ cycloalkylene, C₆₋₁₄ arylene, C₇₋₁₅ aralkylene, heteroarylene, or heterocyclylene; and
   R^{D} is

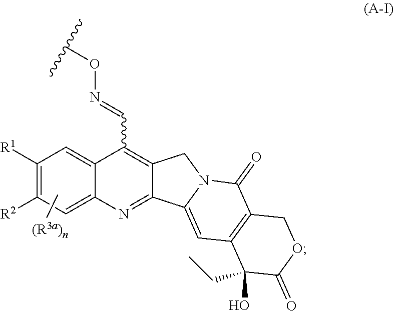

wherein:
R¹ and R² are each independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1C}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c} or —S(O)₂NR^{1b}R^{1c};
each R^{3a} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c} or —S(O)₂NR^{1b}R^{1c};
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and
n is an integer of 0, 1, 2, 3, 4, 5, 6, 7, or 8;
wherein each alkyl, alkylene, heteroalkyl, heteroalkylene, alkenyl, alkenylene, heteroalkenylene, alkynyl, alkynylene, heteroalkynylene, cycloalkyl, cycloalkylene, aryl, arylene, aralkyl, heteroaryl, heteroarylene, heterocyclyl, and heterocyclylene is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{f}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{1b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In another embodiment, provided herein is a compound of Formula (IIA):

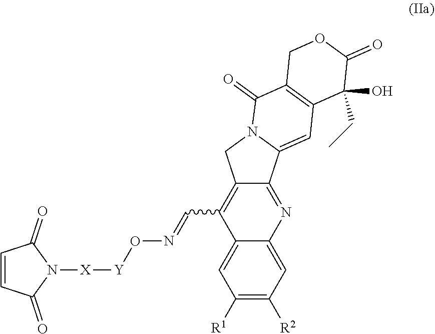

or a diastereomer, a mixture of two or more diastereomers, a tautomer, a mixture of two or more tautomers, or an isotopic variant thereof; or a pharmaceutically acceptable salt, solvate, or hydrate thereof; wherein R¹, R², X, and Y are each as defined herein.

In yet another embodiment, provided herein is a compound of Formula (IIIA):

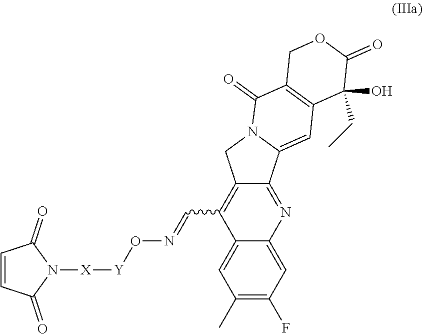

or a diastereomer, a mixture of two or more diastereomers, a tautomer, a mixture of two or more tautomers, or an isotopic variant thereof; or a pharmaceutically acceptable salt, solvate, or hydrate thereof; wherein X and Y are each as defined herein.

The groups, R¹, R², R⁴, R⁶, R⁷, R⁸, R⁹, R¹⁰, R^{3a}, R^{5a}, X, Y, m, n, p, and r in formulae described herein, including Formulae (I) to (VI) and (IA) to (IIIA), are further defined in the embodiments described herein. All combinations of the embodiments provided herein for such groups are within the scope of this disclosure.

In certain embodiments, R¹ is —C(O)R^{1a}, wherein R^{1a} is as defined herein. In certain embodiments, R¹ is —C(O)OR^{1a}, wherein R^{1a} is as defined herein. In certain embodiments, R¹ is —C(O)NR^{1b}R^{1c}, wherein R^{1b} and R^{1c} are each as defined herein. In certain embodiments, R¹ is —C(O)SR^{1a}, wherein R^{1a} is as defined herein. In certain embodiments, R¹ is —C(NR^{1a})NR^{1b}R^{1c}, wherein R^{1a}, R^{1b}, and R^{1c} are each as defined herein. In certain embodiments, R¹ is —C(S)R^{1a}, wherein R^{1a} is as defined herein. In certain embodiments, R¹ is —C(S)OR^{1a}, wherein R^{1a} is as defined herein. In certain embodiments, R¹ is —C(S)NR^{1b}R^{1c}, wherein R^{1b} and R^{1c} are each as defined herein. In certain embodiments, R¹ is —OR^{1a}, wherein R^{1a} is as defined herein. In certain embodiments, R¹ is —OC(O)R^{1a}, wherein R^{1a} is as defined herein. In certain embodiments, R¹ is —OC(O)OR^{1a}, wherein R^{1a} is as defined herein. In certain embodiments, R¹ is —OC(O)NR^{1b}R^{1c}, wherein R^{1b} and R^{1c} are each as defined herein. In certain embodiments, R¹ is —OC(O)SR^{1a}, wherein R^{1a} is as defined herein. In certain embodiments, R¹ is —OC(NR^{1a})NR^{1b}R^{1c}, wherein R^{1a}, R^{1b}, and R^{1c} are each as defined herein. In certain embodiments, R¹ is —OC(S)R^{1a}, wherein R^{1a} is as defined herein. In certain embodiments, R¹ is —OC(S)OR^{1a}, wherein R^{1a} is as defined herein. In certain embodiments, R¹ is —OC(S)NR^{1b}R^{1c}, wherein R^{1b} and R^{1c} are each as defined herein. In certain embodiments, R¹ is —OS(O)R^{1a}, wherein R^{1a} is as defined herein. In certain embodiments, R¹ is-OS(O)₂R^{1a}, wherein R^{1a} is as defined herein. In certain embodiments, R¹ is —OS(O)NR^{1b}R^{1c}, wherein R^{1b} and R^{1c} are each as defined herein. In certain embodiments, R¹ is —OS(O)₂NR^{1b}R^{1c}, wherein R^{1b} and R^{1c} are each as defined herein. In certain embodiments, R¹ is —NR^{1b}R^{1c}, wherein R^{1b} and R^{1c} are each as defined herein. In certain embodiments, R¹ is —NR^{1a}C(O)R^{id}, wherein R^{1a} and R^{1d} are each as defined herein. In certain embodiments, R¹ is —NR^{1a}C(O)OR^{id}, wherein R^{1a} and R^{1d} are each as defined herein. In certain embodiments, R¹ is —NR^{1a}C(O)NR^{1b}R^{1c}, wherein R^{1a}, R^{1b}, and R^{1c} are each as defined herein. In certain embodiments, R¹ is —NR^{1a}C(O)SR^{id}, wherein R^{1a} and R^{1d} are each as defined herein. In certain embodiments, R¹ is —NR^{1a}C(NR^{id})NR^{1b}R^{1c}, wherein R^{1a}, R^{1b}, R^{1c}, and R^{1d} are each as defined herein. In certain embodiments, R¹ is —NR^{1a}C(S)R^{id}, wherein R^{1a} and R^{1d} are each as defined herein. In certain embodiments, R¹ is —NR^{1a}C(S)OR^{id}, wherein R^{1a} and R^{1d} are each as defined herein. In certain embodiments, R¹ is —NR^{1a}C(S)NR^{1b}R^{1c}, wherein R^{1a}, R^{1b}, and R^{1c} are each as defined herein. In certain embodiments, R¹ is —NR^{1a}S(O)R^{id}, wherein R^{1a} and R^{1d} are each as defined herein. In certain embodiments, R¹ is —NR^{1a}S(O)₂R^{1d}, wherein R^{1a} and R^{1d} are each as defined herein. In certain embodiments, R¹ is —NR^{1a}S(O)NR^{1b}R^{1c}, wherein R^{1a}, R^{1b}, and R^{1c} are each as defined herein. In certain embodiments, R¹ is —NR^{1a}S(O)₂NR^{1b}R^{1c}, wherein R^{1a}, R^{1b}, and R^{1c} are each as defined herein. In certain embodiments, R¹ is —SR^{1a}, wherein R^{1a} is as defined herein. In certain embodiments, R¹ is —S(O)R^{1a}, wherein R^{1a} is as defined herein. In certain embodiments, R¹ is —S(O)₂R^{1a}, wherein R^{1a} is as defined herein. In certain embodiments, R¹ is —S(O)NR^{1b}R^{1c}, wherein R^{1b} and R^{1c} are each as defined herein. In certain embodiments, R¹ is —S(O)₂NR^{1b}R^{1c}, wherein R^{1b} and R^{1c} are each as defined herein.

In certain embodiments, R² is hydrogen. In certain embodiments, R² is deuterium. In certain embodiments, R² is cyano. In certain embodiments, R² is halo. In certain embodiments, R² is fluoro or chloro. In certain embodiments, R² is fluoro. In certain embodiments, R² is nitro. In certain embodiments, R² is C₁₋₆ alkyl, optionally substituted with one or more substituents Q. In certain embodiments, R² is methyl. In certain embodiments, R² is C₂₋₆ alkenyl, optionally substituted with one or more substituents Q. In certain embodiments, R² is C₂₋₆ alkynyl, optionally substituted with one or more substituents Q. In certain embodiments, R² is C₃₋₁₀ cycloalkyl, optionally substituted with one or more substituents Q. In certain embodiments, R² is C₆₋₁₄ aryl, optionally substituted with one or more substituents Q. In certain embodiments, R² is C₇₋₁₅ aralkyl, optionally substituted with one or more substituents Q. In certain embodiments, R² is heteroaryl, optionally substituted with one or more substituents Q. In certain embodiments, R² is heterocyclyl, optionally substituted with one or more substituents Q. In certain embodiments, R² is hydrogen or fluoro.

In certain embodiments, R² is —C(O)R^{1a}, wherein R^{1a} is as defined herein. In certain embodiments, R² is —C(O)OR^{1a}, wherein R^{1a} is as defined herein. In certain embodiments, R² is —C(O)NR^{1b}R^{1c}, wherein R^{1b} and R^{1c} are each as defined herein. In certain embodiments, R² is —C(O)SR^{1a}, wherein R^{1a} is as defined herein. In certain embodiments, R² is —C(NR^{1a})NR^{1b}R^{1c}, wherein R^{1a}, R^{1b}, and R^{1c} are each as defined herein. In certain embodiments, R² is —C(S)R^{1a}, wherein R^{1a} is as defined herein. In certain embodiments, R² is —C(S)OR^{1a}, wherein R^{1a} is as defined herein. In certain embodiments, R² is —C(S)NR^{1b}R^{1c}, wherein R^{1b} and R^{1c} are each as defined herein. In certain embodiments, R² is —OR^{1a}, wherein R^{1a} is as defined herein. In certain embodiments, R² is —OC(O)R^{1a}, wherein R^{1a} is as defined herein. In certain embodiments, R² is —OC(O)OR^{1a}, wherein R^{1a} is as defined herein. In certain embodiments, R² is —OC(O)NR^{1b}R^{1c}, wherein R^{1b} and R^{1c} are each as defined herein. In certain embodiments, R² is —OC(O)SR^{1a}, wherein R^{1a} is as defined herein. In certain embodiments, R² is —OC(NR^{1a})NR^{1b}R^{1c}, wherein R^{1a}, R^{1b}, and R^{1c} are each as defined herein. In certain embodiments, R² is —OC(S)R^{1a}, wherein R^{1a} is as defined herein. In certain embodiments, R² is —OC(S)OR^{1a}, wherein R^{1a} is as defined herein. In certain embodiments, R² is —OC(S)NR^{1b}R^{1c}, wherein R^{1b} and R^{1c} are each as defined herein. In certain embodiments, R² is —OS(O)R^{1a}, wherein R^{1a} is as defined herein. In certain embodiments, R² is-OS(O)₂R^{1a}, wherein R^{1a} is as defined herein. In certain embodiments, R² is —OS(O)NR^{1b}R^{1c}, wherein R^{1b} and R^{1c} are each as defined herein. In certain embodiments, R² is —OS(O)₂NR^{1b}R^{1c}, wherein R^{1b} and R^{1c} are each as defined herein. In certain embodiments, R² is —NR^{1b}R^{1c}, wherein R^{1b} and R^{1c} are each as defined herein. In certain embodiments, R² is —NR^{1a}C(O)R^{id}, wherein R^{1a} and R^{id} are each as defined herein. In certain embodiments, R² is —NR^{1a}C(O)OR^{id}, wherein R^{1a} and R^{id} are each as defined herein. In certain embodiments, R² is —NR^{1a}C(O)NR^{1b}R^{1c}, wherein R^{1a}, R^{1b}, and R^{1c} are each as defined herein. In certain embodiments, R² is —NR^{1a}C(O)SR^{id}, wherein R^{1a} and R^{id} are each as defined herein. In certain embodiments, R² is —NR^{1a}C(NR^{id})NR^{1b}R^{1c}, wherein R^{1a}, R^{1b}, R^{1c}, and R^{id} are each as defined herein. In certain embodiments, R² is —NR^{1a}C(S)R^{id}, wherein R^{1a} and R^{id} are each as defined herein. In certain embodiments, R² is —NR^{1a}C(S)OR^{id}, wherein R^{1a} and R^{id} are each as defined herein. In certain embodiments, R² is —NR^{1a}C(S)NR^{1b}R^{1c}, wherein R^{1a}, R^{1b}, and R^{1c} are each as defined herein. In certain embodiments, R² is —NR^{1a}S(O)R^{id}, wherein R^{1a} and R^{id} are each as defined herein. In certain embodiments, R² is —NR^{1a}S(O)₂R^{1d}, wherein R^{1a} and R^{id} are each as defined herein. In certain embodiments, R² is —NR^{1a}S(O)NR^{1b}R^{1c}, wherein R^{1a}, R^{1b}, and R^{1c} are each as defined herein. In certain embodiments, R² is —NR^{1a}S(O)₂NR^{1b}R^{1c}, wherein R^{1a}, R^{1b}, and R^{1c} are each as defined herein. In certain embodiments, R² is —SR^{1a}, wherein R^{1a} is as defined herein. In certain embodiments, R² is —S(O)R^{1a}, wherein R^{1a} is as defined herein. In certain embodiments, R² is —S(O)₂R^{1a}, wherein R^{1a} is as defined herein. In certain embodiments, R² is —S(O)NR^{1b}R^{1c}, wherein R^{1b} and R^{1c} are each as defined herein. In certain embodiments, R² is —S(O)₂NR^{1b}R^{1c}, wherein R^{1b} and R^{1c} are each as defined herein.

In certain embodiments, each R^{3a} is deuterium. In certain embodiments, each R^{3a} is cyano. In certain embodiments, each R^{3a} is independently halo. In certain embodiments, each R^{3a} is independently fluoro or chloro. In certain embodiments, each R^{3a} is fluoro. In certain embodiments, each R^{3a} is nitro. In certain embodiments, each R^{3a} is independently C₁₋₆ alkyl, optionally substituted with one or more substituents Q. In certain embodiments, each R^{3a} is independently methyl. In certain embodiments, each R^{3a} is independently C₂₋₆ alkenyl, optionally substituted with one or more substituents Q. In certain embodiments, each R^{3a} is independently C₂₋₆ alkynyl, optionally substituted with one or more substituents Q. In certain embodiments, each R^{3a} is independently C₃₋₁₀ cycloalkyl, optionally substituted with one or more substituents Q. In certain embodiments, each R^{3a} is independently C₆₋₁₄ aryl, optionally substituted with one or more substituents Q. In certain embodiments, each R^{3a} is independently C₇₋₁₅ aralkyl, optionally substituted with one or more substituents Q. In certain embodiments, each R^{3a} is independently heteroaryl, optionally substituted with one or more substituents Q. In certain embodiments, each R^{3a} is independently heterocyclyl, optionally substituted with one or more substituents Q. In certain embodiments, each R^{3a} is independently hydrogen or fluoro.

In certain embodiments, each R^{3a} is independently —C(O)R^{1a}, wherein R^{1a} is as defined herein. In certain embodiments, each R^{3a} is independently —C(O)OR^{1a}, wherein R^{1a} is as defined herein. In certain embodiments, each R^{3a} is independently —C(O)NR^{1b}R^{1c}, wherein R^{1b} and R^{1c} are each as defined herein. In certain embodiments, each R^{3a} is independently —C(O)SR^{1a}, wherein R^{1a} is as defined herein. In certain embodiments, each R^{3a} is independently —C(NR^{1a})NR^{1b}R^{1c}, wherein R^{1a}, R^{1b}, and R^{1c} are each as defined herein. In certain embodiments, each R^{3a} is independently —C(S)R^{1a}, wherein R^{1a} is as defined herein. In certain embodiments, each R^{3a} is independently —C(S)OR^{1a}, wherein R^{1a} is as defined herein. In certain embodiments, each R^{3a} is independently —C(S)NR^{1b}R^{1c}, wherein R^{1b} and R^{1c} are each as defined herein. In certain embodiments, each R^{3a} is independently —OR^{1a}, wherein R^{1a} is as defined herein. In certain embodiments, each R^{3a} is independently —OC(O)R^{1a}, wherein R^{1a} is as defined herein. In certain embodiments, each R^{3a} is independently —OC(O)OR^{1a}, wherein R^{1a} is as defined herein. In certain embodiments, each R^{3a} is independently —OC(O)NR^{1b}R^{1c}, wherein R^{1b} and R^{1c} are each as defined herein. In certain embodiments, each R^{3a} is independently —OC(O)SR^{1a}, wherein R^{1a} is as defined herein. In certain embodiments, each R^{3a} is independently —OC(NR^{1a})NR^{1b}R^{1c}, wherein R^{1a}, R^{1b}, and R^{1c} are each as defined herein. In certain embodiments, each R^{3a} is independently —OC(S)R^{1a}, wherein R^{1a} is as defined herein. In certain embodiments, each R^{3a} is independently —OC(S)OR^{1a}, wherein R^{1a} is as defined herein. In certain embodiments, each R^{3a} is independently —OC(S)NR^{1b}R^{1c}, wherein R^{1b} and R^{1c} are each as defined herein. In certain embodiments, each R^{3a} is independently —OS(O)R^{1a}, wherein R^{1a} is as defined herein. In certain embodiments, each R^{3a} is independently-OS(O)₂R^{1a}, wherein R^{1a} is as defined herein. In certain embodiments, each R^{3a} is independently —OS(O)NR^{1b}R^{1c}, wherein R^{1b} and R^{1c} are each as defined herein. In certain embodiments, each R^{3a} is independently —OS(O)₂NR^{1b}R^{1c}, wherein R^{1b} and R^{1c} are each as defined herein. In certain embodiments, each R^{3a} is independently —NR^{1b}R^{1c}, wherein R^{1b} and R^{1c} are each as defined herein. In certain embodiments, each R^{3a} is independently —NR^{1a}C(O)R^{1d}, wherein R^{1a} and R^{1d} are each as defined herein. In certain embodiments, each R^{3a} is independently —NR^{1a}C(O)OR^{1d}, wherein R^{1a} and R^{1d} are each as defined herein. In certain embodiments, each R^{3a} is independently —NR^{1a}C(O)NR^{1b}R^{1c}, wherein R^{1a}, R^{1b}, and R^{1c} are each as defined herein. In certain embodiments, each R^{3a} is independently —NR^{1a}C(O)SR^{1d}, wherein R^{1a} and R^{1d} are each as defined herein. In certain embodiments, each R^{3a} is independently —NR^{1a}C(NR^{1d})NR^{1b}R^{1c}, wherein R^{1a}, R^{1b}, R^{1c}, and R^{1d} are each as defined herein. In certain embodiments, each R^{3a} is independently —NR^{1a}C(S)R^{1d}, wherein R^{1a} and R^{1d} are each as defined herein. In certain embodiments, each R^{3a} is independently —NR^{1a}C(S)OR^{1d}, wherein R^{1a} and R^{1d} are each as defined herein. In certain embodiments, each R^{3a} is independently —NR^{1a}C(S)NR^{1b}R^{1c}, wherein R^{1a}, R^{1b}, and R^{1c} are each as defined herein. In certain embodiments, each R^{3a} is independently —NR^{1a}S(O)R^{1d}, wherein R^{1a} and R^{1d} are each as defined herein. In certain embodiments, each R^{3a} is independently —NR^{1a}S(O)₂R^{1d}, wherein R^{1a} and R^{1d} are each as defined herein. In certain embodiments, each R^{3a} is independently —NR^{1a}S(O)NR^{1b}R^{1c}, wherein R^{1a}, R^{1b}, and R^{1c} are each as defined herein. In certain embodiments, each R^{3a} is independently —NR^{1a}S(O)₂NR^{1b}R^{1c}, wherein R^{1a}, R^{1b}, and R^{1c} are each as defined herein. In certain embodiments, each R^{3a} is independently —SR^{1a}, wherein R^{1a} is as defined herein. In certain embodiments, each R^{3a} is independently —S(O)R^{1a}, wherein R^{1a} is as defined herein. In certain embodiments, each R^{3a} is independently —S(O)₂R^{1a}, wherein R^{1a} is as defined herein. In certain embodiments, each R^{3a} is independently —S(O)NR^{1b}R^{1c}, wherein R^{1b} and R^{1c} are each as defined herein. In certain embodiments, each R^{3a} is independently —S(O)₂NR^{1b}R^{1c}, wherein R^{1b} and R^{1c} are each as defined herein.

In certain embodiments, each X is independently C₁₋₄₀ alkylene, optionally substituted with one or more substituents Q. In certain embodiments, each X is independently C₁₋₃₀ alkylene, optionally substituted with one or more substituents Q. In certain embodiments, each X is independently C₁₋₂₀ alkylene, optionally substituted with one or more substituents Q. In certain embodiments, each X is independently C₁₋₄₀ heteroalkylene, optionally substituted with one or more substituents Q. In certain embodiments, each X is independently C₁₋₃₀ heteroalkylene, optionally substituted with one or more substituents Q. In certain embodiments, each X is independently C₁₋₂₀ heteroalkylene, optionally substituted with one or more substituents Q. In certain embodiments, each X is independently C₂₋₄₀ alkenylene, optionally substituted with one or more substituents Q. In certain embodiments, each X is independently C₂₋₄₀ heteroalkenylene, optionally substituted with one or more substituents Q. In certain embodiments, each X is independently C₂₋₄₀ alkynylene, optionally substituted with one or more substituents Q. In certain embodiments, each X is independently C₂₋₄₀ heteroalkynylene, optionally substituted with one or more substituents Q. In certain embodiments, each X is independently C₃₋₁₀ cycloalkylene, optionally substituted with one or more substituents Q. In certain embodiments, each X is independently C₆₋₁₄ arylene, optionally substituted with one or more substituents Q. In certain embodiments, each X is independently heteroarylene, optionally substituted with one or more substituents Q. In certain embodiments, each X is independently heterocyclylene, optionally substituted with one or more substituents Q.

In certain embodiments, each X is independently C₁₋₄₀ alkylene, wherein one or more methylene groups are each independently and optionally replaced by a divalent group, and each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q. In certain embodiments, each X is independently C₁₋₃₀ alkylene, wherein one or more methylene groups are each independently and optionally replaced by a divalent group, and each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q. In certain embodiments, each X is independently C₁₋₂₀ alkylene, wherein one or more methylene groups are each independently and optionally replaced by a divalent group, and each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q.

In certain embodiments, each X is independently C₁₋₄₀ heteroalkylene, wherein one or more methylene groups are each independently and optionally replaced by a divalent group, and each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q. In certain embodiments, each X is independently C₁₋₃₀ heteroalkylene, wherein one or more methylene groups are each independently and optionally replaced by a divalent group, and each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q. In certain embodiments, each X is independently C₁₋₂₀ heteroalkylene, wherein one or more methylene groups are each independently and optionally replaced by a divalent group, and each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q.

In certain embodiments, each X is independently C₂₋₄₀ alkenylene, wherein one or more methylene groups are each independently and optionally replaced by a divalent group, and each divalent group is independently C₃₋₁₀ cycloalkylene, C₆₋₁₄ arylene, heteroarylene, or heterocyclylene; and wherein the alkenylene, cycloalkylene, arylene, heteroarylene, and heterocyclylene are each optionally substituted with one or more substituents Q. In certain embodiments, each X is independently C₂₋₄₀ heteroalkenylene, wherein one or more methylene groups are each independently and optionally replaced by a divalent group, and each divalent group is independently C₃₋₁₀ cycloalkylene, C₆₋₁₄ arylene, heteroarylene, or heterocyclylene; and wherein the heteroalkenylene, cycloalkylene, arylene, heteroarylene, and heterocyclylene are each optionally substituted with one or more substituents Q. In certain embodiments, each X is independently C₂₋₄₀ alkynylene, wherein one or more methylene groups are each independently and optionally replaced by a divalent group, and each divalent group is independently C₃₋₁₀ cycloalkylene, C₆₋₁₄ arylene, heteroarylene, or heterocyclylene; and wherein the alkynylene, cycloalkylene, arylene, heteroarylene, and heterocyclylene are each optionally substituted with one or more substituents Q. In certain embodiments, each X is independently C₂₋₄₀ heteroalkynylene, wherein one or more methylene groups are each independently and optionally replaced by a divalent group, and each divalent group is independently C₃₋₁₀ cycloalkylene, C₆₋₁₄ arylene, heteroarylene, or heterocyclylene; and wherein the heteroalkynylene, cycloalkylene, arylene, heteroarylene, and heterocyclylene are each optionally substituted with one or more substituents Q. In certain embodiments, each X is

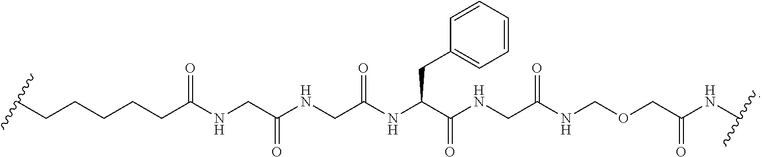 [SMILES: *CCCCCC(=O)NCC(=O)NCC(=O)N[C@@H](Cc1ccccc1)C(=O)NCC(=O)NCOCC(=O)N*]

In certain embodiments, each Y is a bond. In certain embodiments, each Y is independently C₁₋₆ alkylene, optionally substituted with one or more substituents Q. In certain embodiments, each Y is independently —(CH₂)ᵣ—, wherein r is an integer of 1, 2, 3, 4, 5, or 6. In certain embodiments, each Y is independently ethanediyl, propanediyl, or butanediyl. In certain embodiments, each Y is independently ethane-1,1-diyl, propane-1,3-diyl, or 2-methylpropane-1,3-diyl. In certain embodiments, each Y is independently C₁₋₆ heteroalkylene, optionally substituted with one or more substituents Q. In certain embodiments, each Y is independently C₂₋₆ alkenylene, optionally substituted with one or more substituents Q. In certain embodiments, each Y is independently C₂₋₆ alkynylene, optionally substituted with one or more substituents Q. In certain embodiments, each Y is independently C₃₋₁₀ cycloalkylene, optionally substituted with one or more substituents Q. In certain embodiments, each Y is independently C₆₋₁₄ arylene, optionally substituted with one or more substituents Q. In certain embodiments, each Y is independently C₇₋₁₅ aralkylene, optionally substituted with one or more substituents Q. In certain embodiments, each Y is independently heteroarylene, optionally substituted with one or more substituents Q. In certain embodiments, each Y is independently heterocyclylene, optionally substituted with one or more substituents Q.

In certain embodiments, m is an integer of 1, 2, 3, 4, 5, 6, 7, 8, 9, 10, 11, or 12. In certain embodiments, m is an integer of 1, 2, 3, 4, 5, 6, 7, 8, 9, or 10. In certain embodiments, m is an integer of 1, 2, 3, 4, 5, 6, 7, or 8. In certain embodiments, m is an integer of 1. In certain embodiments, m is an integer of 2. In certain embodiments, m is an integer of 3. In certain embodiments, m is an integer of 4. In certain embodiments, m is an integer of 5. In certain embodiments, m is an integer of 6. In certain embodiments, m is an integer of 7. In certain embodiments, m is an integer of 8. In certain embodiments, m is an integer of 9. In certain embodiments, m is an integer of 10. In certain embodiments, m is an integer of 11. In certain embodiments, m is an integer of 12. In certain embodiments, m is an integer of 11. In certain embodiments, m is an integer of 13. In certain embodiments, m is an integer of 11. In certain embodiments, m is an integer of 14. In certain embodiments, m is an integer of 11. In certain embodiments, m is an integer of 15. In certain embodiments, m is an integer of 11. In certain embodiments, m is an integer of 16.

In certain embodiments, each n is independently an integer of 0. In certain embodiments, each n is independently an integer of 1. In certain embodiments, each n is independently an integer of 2. In certain embodiments, each n is independently an integer of 3. In certain embodiments, each n is independently an integer of 4. In certain embodiments, each n is independently an integer of 5. In certain embodiments, each n is independently an integer of 6. In certain embodiments, each n is independently an integer of 7. In certain embodiments, each n is independently an integer of 8.

In one embodiment, provided herein is a compound of:

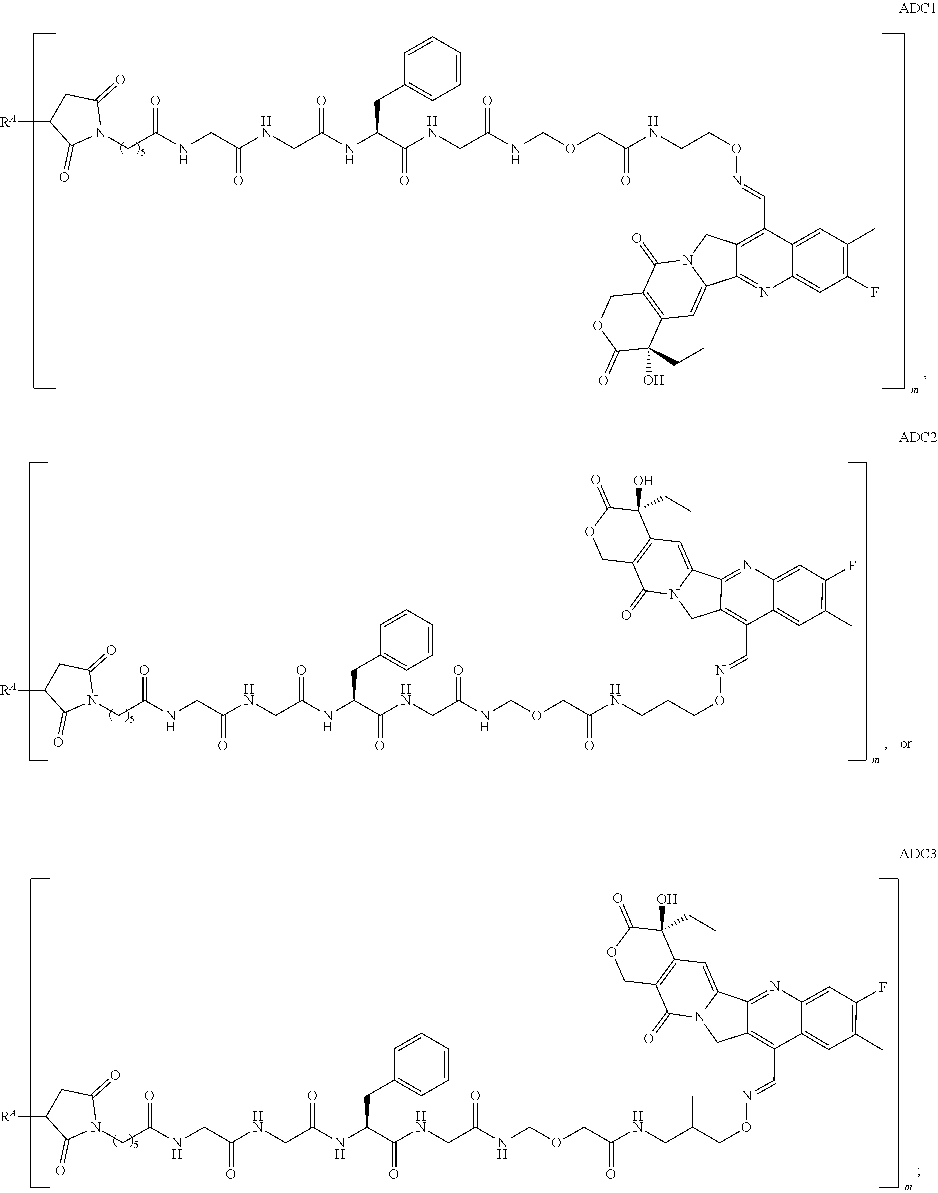

or a diastereomer, a mixture of two or more diastereomers, a tautomer, a mixture of two or more tautomers, or an isotopic variant thereof; or a pharmaceutically acceptable salt, solvate, or hydrate thereof; wherein, in one embodiment, each R^{A} is independently an antibody, in one embodiment, an anti-B7H3 antibody, anti-CD20 antibody, anti-FOLR1 antibody, anti-HER2 antibody, anti-MSLN antibody, anti-TROP2 antibody; and each m is as defined herein, in another embodiment, each m is independently an integer of 2, 3, 4, 5, 6, 7, or 8; and wherein each linker is independently connected covalently to a thiol group of a cysteine residue of the antibody.

In certain embodiments, a compound provided herein in a composition (e.g., a pharmaceutical composition) has a drug-antibody ratio (DAR) ranging from about 0.5 to about 12, from about 1 to about 10, from about 2 to about 8, or from about 3 to about 5. In certain embodiments, a compound provided herein in a composition (e.g., a pharmaceutical composition) has a DAR ranging from about 0.5 to about 12. In certain embodiments, a compound provided herein in a composition (e.g., a pharmaceutical composition) has a DAR ranging from about 1 to about 10. In certain embodiments, a compound provided herein in a composition (e.g., a pharmaceutical composition) has a DAR ranging from about 2 to about 8. In certain embodiments, a compound provided herein in a composition (e.g., a pharmaceutical composition) has a DAR ranging from about 3 to about 5. In certain embodiments, a compound provided herein in a composition (e.g., a pharmaceutical composition) has a DAR of about 2, about 2.5, about 3, about 3.5, about 4, about 4.5, about 5, about 5.5, about 6, about 6.5, about 7, about 7.5, or about 8.

In one embodiment, provided herein is:
N—((S,E/Z)-16-benzyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-7,12,15,18,21-pentaoxo-3,9-dioxa-2,6,11,14,17,20-hexaazadocos-1-en-22-yl)-6-(2,5-dioxo-2,5-dihydro-1H-pyrrol-1-yl)hexanamide A1;
N—((S,E/Z)-17-benzyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-8,13,16,19,22-pentaoxo-3,10-dioxa-2,7,12,15,18,21-hexaazatricos-1-en-23-yl)-6-(2,5-dioxo-2,5-dihydro-1H-pyrrol-1-yl)hexanamide A2; or
N-((17S,E/Z)-17-benzyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-5-methyl-8,13,16,19,22-pentaoxo-3,10-dioxa-2,7,12,15,18,21-hexaazatricos-1-en-23-yl)-6-(2,5-dioxo-2,5-dihydro-1H-pyrrol-1-yl)hexanamide A3;
or a diastereomer, a mixture of two or more diastereomers, a tautomer, a mixture of two or more tautomers, or an isotopic variant thereof; or a pharmaceutically acceptable salt, solvate, or hydrate thereof.

In another embodiment, provided herein is:
(S,E/Z)—N-(2-((((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methylene)amino)oxy)ethyl)-2-hydroxyacetamide B1;
(S,E/Z)—N-(3-((((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methylene)amino)oxy)propyl)-2-hydroxyacetamide B2; or
N-(3-((((E/Z)—((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methylene)amino)oxy)-2-methyl-propyl)-2-hydroxyacetamide B3;
or a diastereomer, a mixture of two or more diastereomers, a tautomer, a mixture of two or more tautomers, or an isotopic variant thereof; or a pharmaceutically acceptable salt, solvate, or hydrate thereof.

In yet another embodiment, provided herein is a trastuzumab ADC selected from ADC-A1, ADC-A2, and ADC-A3.

In certain embodiments, a compound provided herein is deuterium-enriched. In certain embodiments, a compound provided herein is carbon-13 enriched. In certain embodiments, a compound provided herein is carbon-14 enriched. In certain embodiments, a compound provided herein contains one or more less prevalent isotopes for other elements, including, but not limited to, ¹⁵N for nitrogen; ¹⁷O or ¹⁸O for oxygen, and ³⁴S, ³⁵S, or ³⁶S for sulfur.

In certain embodiments, a compound provided herein is isolated or purified. In certain embodiments, a compound provided herein has a purity of at least about 90%, at least about 95%, at least about 98%, at least about 99%, or at least about 99.5% by weight.

The compounds provided herein are intended to encompass all possible stereoisomers, unless a particular stereochemistry is specified. Where a compound provided herein contains an alkenyl group, the compound may exist as one or mixture of geometric cis/trans (or Z/E) isomers. Where structural isomers are interconvertible, the compound may exist as a single tautomer or a mixture of tautomers. This can take the form of proton tautomerism in the compound that contains, for example, an imino, keto, or oxime group; or so-called valence tautomerism in the compound that contains an aromatic moiety. It follows that a single compound may exhibit more than one type of isomerism.

A compound provided herein can be enantiomerically pure, such as a single enantiomer or a single diastereomer, or be stereoisomeric mixtures, such as a mixture of enantiomers, e.g., a racemic mixture of two enantiomers; or a mixture of two or more diastereomers. As such, one of ordinary skill in the art will recognize that administration of a compound in its (R) form is equivalent, for the compound that undergoes epimerization in vivo, to administration of the compound in its (S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

When a compound provided herein contains an acidic or basic moiety, it can also be provided as a pharmaceutically acceptable salt. See, Berge et al., J. Pharm. Sci. 1977, 66, 1-19; Handbook of Pharmaceutical Salts: Properties, Selection, and Use, 2nd ed.; Stahl and Wermuth Eds.; John Wiley & Sons, 2011. In certain embodiments, a pharmaceutically acceptable salt of a compound provided herein is a solvate. In certain embodiments, a pharmaceutically acceptable salt of a compound provided herein is a hydrate.

Suitable acids for use in the preparation of pharmaceutically acceptable salts of a compound provided herein include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts of a compound provided herein include, but are not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including, but not limited to, L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, N-methyl-D-glucamine, 2-amino-2-(hydroxymethyl)-1,3-propanediol, and tromethamine.

A compound provided herein may also be provided as a prodrug, which is a functional derivative of the compound and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis.

### Method of Synthesis

In one embodiment, provided herein is a method of synthesizing compound b, comprising the step of contacting compound a with a solvent in the presence of air; wherein R¹, R², R^{3a}, and n are each as defined herein. In certain embodiments, the solvent is acetic acid. In certain embodiments, the contacting step is performed at a temperature ranging from about 50 to 150° C.

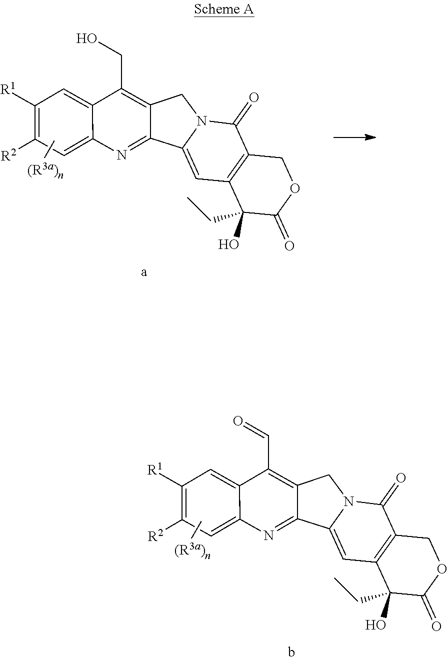

In another embodiment, provided herein is a method of synthesizing (S)-4-ethyl-8-fluoro-4-hydroxy-9-methyl-3,14-dioxo-3,4,12,14-tetrahydro-1H-pyrano[3′,4′:6,7]indolizino-[1,2-b]quinoline-11-carbaldehyde, comprising the step of contacting (S)-4-ethyl-8-fluoro-4-hydroxy-11-(hydroxymethyl)-9-methyl-1,12-dihydro-14H-pyrano[3′,4′:6,7]indolizino[1,2-b]quinoline-3,14(4H)-dione with a solvent in the presence of air. In certain embodiments, the solvent is acetic acid. In certain embodiments, the contacting step is performed at a temperature ranging from about 50 to 150° C.

### Pharmaceutical Compositions

In one embodiment, provided herein is a pharmaceutical composition, comprising a compound provided herein, e.g., a compound of Formula (I), or a diastereomer, a mixture of two or more diastereomers, a tautomer, a mixture of two or more tautomers, or an isotopic variant thereof; or a pharmaceutically acceptable salt, solvate, hydrate, or prodrug thereof; and a pharmaceutically acceptable excipient.

In one embodiment, the pharmaceutical composition provided herein is formulated in a dosage form for parenteral administration. In another embodiment, the pharmaceutical composition provided herein is formulated in a dosage form for intravenous administration. In yet another embodiment, the pharmaceutical composition provided herein is formulated in a dosage form for intramuscular administration. In still another embodiment, the pharmaceutical composition provided herein is formulated in a dosage form for subcutaneous administration.

The pharmaceutical composition provided herein can be provided in a unit-dosage form or multiple-dosage form. A unit-dosage form, as used herein, refers to physically discrete a unit suitable for administration to a subject, and packaged individually as is known in the art. Each unit-dose contains a predetermined quantity of an active ingredient(s) (e.g., a compound provided herein) sufficient to produce the desired therapeutic effect, in association with the required pharmaceutical excipient(s). Examples of a unit-dosage form include, but are not limited to, an ampoule and syringe. A unit-dosage form may be administered in fractions or multiples thereof. A multiple-dosage form is a plurality of identical unit-dosage forms packaged in a single container to be administered in a segregated unit-dosage form. Examples of a multiple-dosage form include, are not limited to, a vial or bottle of pints or gallons.

The pharmaceutical composition provided herein can be administered at once or multiple times at intervals of time. It is understood that the precise dosage and duration of treatment may vary with the age, weight, and condition of the subject being treated, and may be determined empirically using known testing protocols or by extrapolation from in vivo or in vitro test or diagnostic data. It is further understood that for any particular individual, specific dosage regimens should be adjusted over time according to the subject's need and the professional judgment of the person administering or supervising the administration of the pharmaceutical composition.

### Methods of Use

In one embodiment, provided herein is a method of treating, preventing, or ameliorating one or more symptoms of a proliferative disease in a subject, comprising administering to the subject in need thereof a therapeutically effective amount of a compound provided herein, e.g., a compound of Formula (I), or a diastereomer, a mixture of two or more diastereomers, a tautomer, a mixture of two or more tautomers, or an isotopic variant thereof; or a pharmaceutically acceptable salt, solvate, hydrate, or prodrug thereof.

In certain embodiments, the proliferative disease is cancer. In certain embodiments, the cancer is a solid tumor. In certain embodiments, the cancer is a hematologic malignancy.

In certain embodiments, the cancer is breast cancer, colorectal cancer, epidermoid carcinoma, gastric cancer, leukemia, lung cancer, lymphoma, melanoma, oral cancer, ovarian cancer, or pancreatic cancer. In certain embodiments, the cancer is breast cancer, B cell leukemia, Burkitt's lymphoma, colorectal cancer, epidermoid carcinoma, gastric cancer, lung cancer, melanoma, oral cancer, ovarian cancer, pancreatic cancer, T cell leukemia, or T cell lymphoma.

In certain embodiments, the cancer is refractory and/or relapsed. In certain embodiments, the cancer is refractory. In certain embodiments, the cancer is relapsed. In certain embodiments, the cancer is metastatic. In certain embodiments, the cancer is resectable. In certain embodiments, the cancer is unresectable. In certain embodiments, the cancer is metastatic.

In certain embodiments, the cancer is drug-resistant. In certain embodiment, the cancer is multidrug-resistant. In certain embodiments, the cancer is resistant to a chemotherapy. In certain embodiments, the cancer is resistant to an immunotherapy. In certain embodiments, the cancer is resistant to a standard therapy for the cancer.

In certain embodiments, the subject is a mammal. In certain embodiments, the subject is a human.

In certain embodiments, the therapeutically effective amount of a compound provided herein is ranging from about 0.1 mg/kg every four weeks to about 20 mg/kg every week, from about 0.2 mg/kg every four weeks to about 10 mg/kg every week, from about 0.5 mg/kg every four weeks to about 5 mg/kg every week, or from about 1 mg/kg every four weeks to about 2 mg/kg every week.

In one embodiment, the therapeutically effective amount of a compound provided herein is ranging from about 0.1 mg/kg every four weeks to about 20 mg/kg every week. In another embodiment, the therapeutically effective amount of a compound provided herein is ranging from about 0.2 mg/kg every four weeks to about 10 mg/kg every week. In yet another embodiment, the therapeutically effective amount of a compound provided herein is ranging from about 0.5 mg/kg every four weeks to about 5 mg/kg every week. In yet another embodiment, the therapeutically effective amount of a compound provided herein is ranging from about 1 mg/kg every four weeks to about 2 mg/kg every week. In still another embodiment, the therapeutically effective amount of a compound provided herein is about 1, about 1.5, about 2, about 2.5, about 3, about 3.5, about 4, about 4.5, or about 5 mg/kg every three weeks.

Depending on the disorder, disease, or condition to be treated and the subject's condition, a compound provided herein may be administered by parenteral (e.g., intramuscular, intraperitoneal, intravenous, CIV, intracisternal injection or infusion, subcutaneous injection, or implant) routes of administration. A compound provided herein may be formulated in suitable dosage unit with a pharmaceutically acceptable excipient, carrier, adjuvant, or vehicle, appropriate for the route of administration.

In one embodiment, a compound provided herein is administered parenterally. In another embodiment, a compound provided herein is administered intravenously. In yet another embodiment, a compound provided herein is administered intramuscularly. In still another embodiment, a compound provided herein is administered subcutaneously.

A compound provided herein can be delivered as a single dose such as, e.g., a single bolus injection, or over time such as, e.g., continuous infusion over time or divided bolus doses over time. A compound provided herein can be administered repetitively if necessary, for example, until the subject experiences stable disease or regression, or until the patient experiences disease progression or unacceptable toxicity.

A compound provided herein can be administered once a week, once every two weeks, once every three weeks, once every four weeks, once every five weeks, or once every six weeks. In one embodiment, a compound provided herein is administered once a week. In another embodiment, a compound provided herein is administered once every two weeks. In yet another embodiment, a compound provided herein is administered once every two weeks. In yet another embodiment, a compound provided herein is administered once every three weeks. In yet another embodiment, a compound provided herein is administered once every four weeks. In yet another embodiment, a compound provided herein is administered once every five weeks. In yet another embodiment, a compound provided herein is administered once every six weeks.

In certain embodiments, a compound provided herein is cyclically administered to a subject. Cycling therapy involves the administration of an active agent for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment.

A compound provided herein can also be combined or used in combination with other therapeutic agents useful in the treatment and/or prevention of a condition, disorder, or disease described herein.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein) can be administered prior to (e.g., 5 minutes, 15 minutes, 50 minutes, 65 minutes, 1 hour, 2 hours, 6 hours, 6 hours, 12 hours, 26 hours, 68 hours, 72 hours, 96 hours, 1 week, 2 weeks, 3 weeks, 4 weeks, 8 weeks, or 12 weeks before), concomitantly with, or subsequent to (e.g., 5 minutes, 15 minutes, 50 minutes, 65 minutes, 1 hour, 2 hours, 6 hours, 12 hours, 26 hours, 68 hours, 72 hours, 96 hours, 1 week, 2 weeks, 3 weeks, 4 weeks, 8 weeks, or 12 weeks after) the administration of a second therapy (e.g., a prophylactic or therapeutic agent) to the subject. Triple therapy is also contemplated herein.

The route of administration of a compound provided herein is independent of the route of administration of a second therapy. In one embodiment, a compound provided herein is administered intravenously. Thus, in accordance with certain embodiments, a compound provided herein is administered intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraocularly, via local delivery by catheter or stent, subcutaneously, intraadiposally, intraarticularly, intrathecally, or in a slow release dosage form. In one embodiment, a compound provided herein and a second therapy are administered by the same mode of administration intravenously. In another embodiment, a compound provided herein is administered by one mode of administration, e.g., intravenously, whereas the second agent (an anticancer agent) is administered by another mode of administration, e.g., orally.

In one embodiment, provided herein is a method of inhibiting the growth of a cell, comprising contacting the cell with a compound provided herein, e.g., a compound of Formula (I), or an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug thereof.

In certain embodiments, the cell is a cancerous cell. In certain embodiments, the cell is a human cell. In certain embodiments, the cell is a human cancerous cell.

A compound provided herein can also be provided as an article of manufacture using packaging materials well known to those of skill in the art. See, e.g., U.S. Pat. Nos. 5,525,907; 5,052,558; and 5,055,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

In certain embodiments, provided herein is a kit which, when used by a medical practitioner, can simplify the administration of an appropriate amount of a compound provided herein as an active ingredient to a subject. In certain embodiments, the kit provided herein includes a container and a dosage form of a compound provided herein.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, water for injection USP, sodium chloride injection, Ringer's injection, dextrose injection, dextrose and sodium chloride injection, and lactated Ringer's injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

The disclosure will be further understood by the following non-limiting examples.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society, the Journal of Medicinal Chemistry, or the Journal of Biological Chemistry. Specifically, but without limitation, the following abbreviations may be used in the examples and throughout the specification: g (grams); mg (milligrams); mL (milliliters); L (microliters); mM (millimolar); μM (micromolar); mmol (millimoles); h (hour or hours); min (minute or minutes); ACN (acetonitrile); DCM (dichloromethane); DMF (dimethylformamide); DMSO (dimethyl sulfoxide); EtOAc (ethyl acetate); EtOH (ethanol); MeOH (methanol); THF (tetrahydrofuran); DIPEA (N,N-diisopropylethylamine); DMTMM(4-(4,6-dimethoxy-1,3,5-triazin-2-yl)-4-methylmorpholinium chloride); HOAc (acetic acid); pTsOH (p-toluenesulfonic acid); TCEP (tris(2-carboxyethyl)phosphine); HPLC (high-performance liquid chromatography); MS (mass spectrometry); and NMR (nuclear magnetic resonance).

For all of the following examples, standard work-up and purification methods known to those skilled in the art can be utilized. Unless otherwise indicated, all temperatures are expressed in ° C. (degrees Centigrade). All reactions are conducted at room temperature unless otherwise specified. Synthetic methodologies illustrated herein are intended to exemplify the applicable chemistry through the use of specific examples and are not indicative of the scope of the disclosure.

### Example 1

### Synthesis of N—((S,E/Z)—17-benzyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-8,13,16,19,22-pentaoxo-3,10-dioxa-2,7,12,15,18,21-hexaazatricos-1-en-23-yl)-6-(2,5-dioxo-2,5-dihydro-1H-pyrrol-1-yl)hexanamide A2

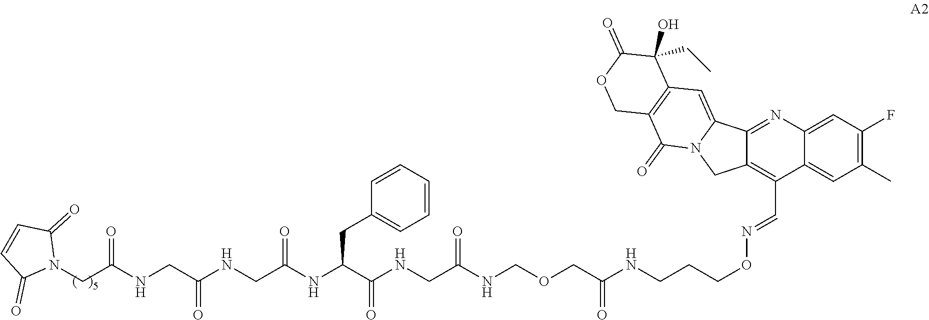

Compound A2 was synthesized as shown in Scheme 1.

(E)-N-(3-Fluoro-4-methylphenyl)-2-(hydroxyimino)acetamide 2. To a mixture of sodium sulfate (240 g) and chloral hydrate (30.6 g) in water (670 mL) stirred at 20-30° C. were added 3-fluoro-4-methylaniline 1 (21 g), 1N HCl (250 mL), and hydroxylamine hydrochloride (47 g). After heated at 80±5° C. overnight and cooled to 5±5° C., the mixture was filtered to yield a solid, which was dried at 60±5° C. overnight to afford compound 2 (26.4 g) in 80% yield. LCMS (ESI) m/z: 197 [M+H]⁺.

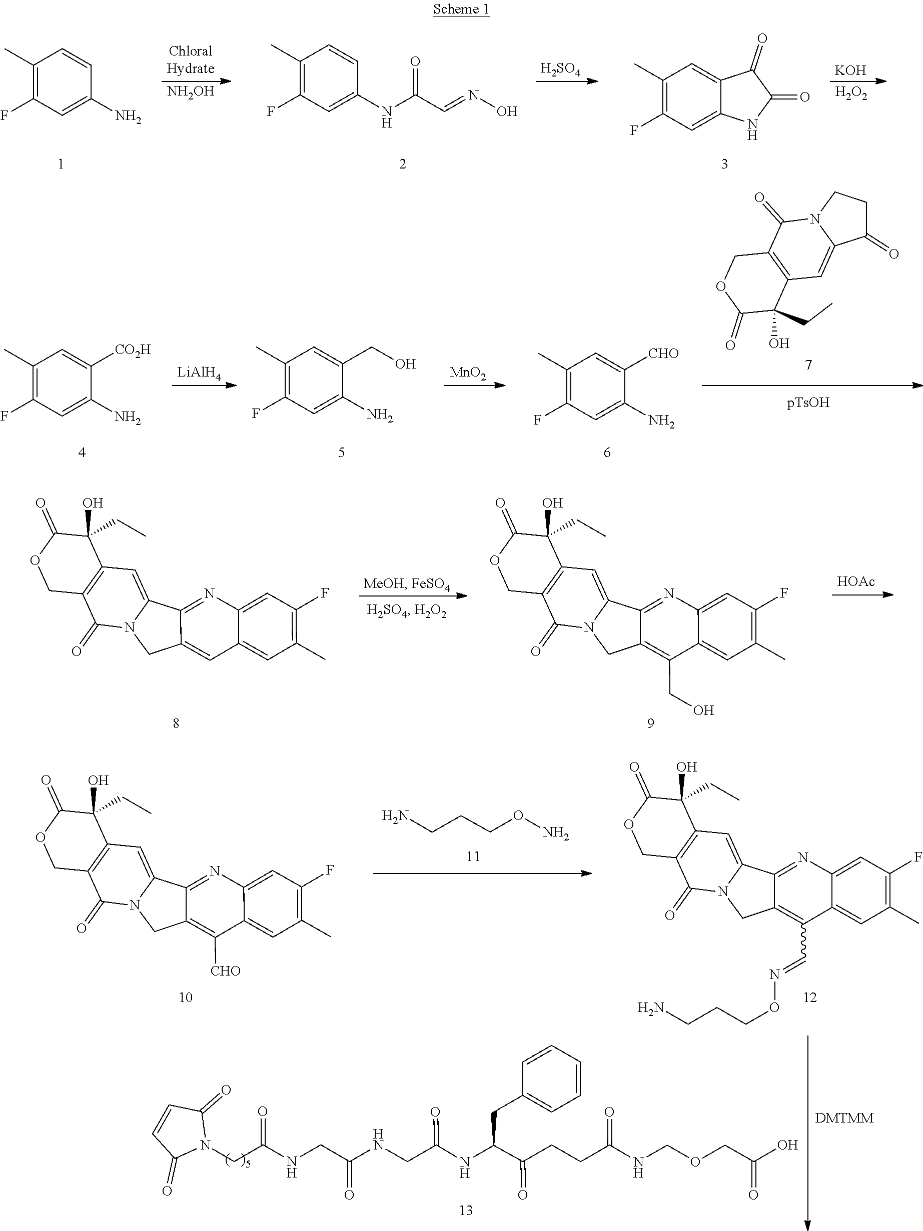

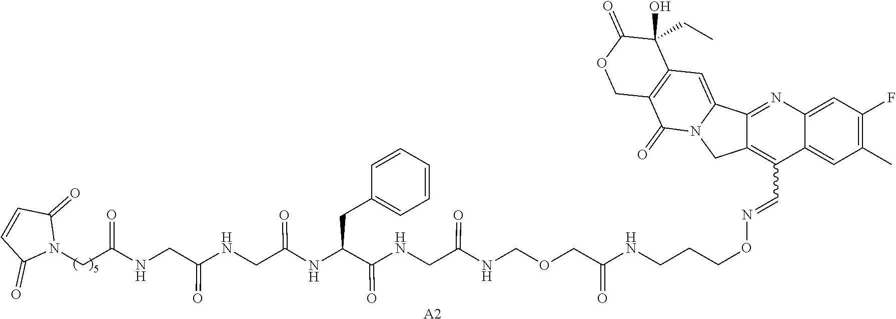

6-Fluoro-5-methylindoline-2,3-dione 3. To 98% sulfuric acid (240 mL) at 20-30° C. was added compound 2 (20 g) in portions. After heated at 80±5° C. for 4 h and cooled to 20±5° C., the mixture was poured into water (480 mL) while keeping the temperature below 50° C. The mixture was cooled to 5±5° C. and filtered to yield a solid, which was dissolved in aqueous NaOH solution (1N, 300 mL). The mixture was adjusted to pH 8˜9 with HOAc and filtered. The filtrate was adjusted with concentrated HCl until pH<2 at a temperature, cooled to 5±5° C., and filtered to yield a solid, which was dried at 60±5° C. for 16 h to afford compound 3 (20 g). LCMS (ESI) m/z: 180 [M+H]⁺.

2-Amino-4-fluoro-5-methylbenzoic acid 4. To a mixture of compound 3 (20 g), KOH (6.9 g), and KCl (17.6 g) in water (400 mL) stirred at 0˜10° C. was added 30% hydrogen peroxide solution (20 g) dropwise. After stirred at 20-30° C. overnight, the mixture was cooled to 10±5° C. and adjusted to pH 4-6 with HOAc. The mixture was cooled to 5±5° C., stirred for 1 h, and then filtered to yield a solid, which was dried at 60±5° C. for 16 h to afford compound 4 (10.2 g) in 58% yield for the last two steps. ¹H NMR (400 MHz, DMSO-d₆) δ 7.55 (d, J=8.2 Hz, 1H), 6.34 (d, J=12.1 Hz, 1H), 2.02 (s, 3H); LCMS (ESI) m/z: 170 [M+H]⁺.

(2-Amino-4-fluoro-5-methylphenyl)methanol 5. To a mixture of LiAlH₄ (5.7 g) in THF (100 mL) at 0˜10° C. under N2 was added a solution of compound 4 (10 g) in THF (100 mL) slowly. After the mixture was stirred at room temperature overnight and cooled to 0˜10° C., water (300 mL) was added carefully, followed by addition of EtOAc (300 mL). The organic layer was separated and the aqueous layer was extracted with EtOAc. The combined organic layers were washed with brine, dried over anhydrous Na₂SO₄, and evaporated to dryness to afford compound 5 (8.3 g) in 91% yield. ¹H NMR (600 MHz, DMSO-d₆) δ 6.90 (d, J=9.0 Hz, 1H), 6.37 (d, J=12.1 Hz, 1H), 4.98 (s, 2H), 4.95 (t, J=5.6 Hz, 10H), 4.32 (d, J=5.1 Hz, 2H), 2.05 (s, 3H); LCMS (ESI) m/z: 156 [M+H]⁺.

2-Amino-4-fluoro-5-methylbenzaldehyde 6. To a mixture of compound 5 (8.3 g) and DCM (166 mL) at 20-30° C. was added MnO₂ (24 g). After stirred at room temperature overnight, the mixture was filtered and the solid was washed with DCM. The filtrate was evaporated to dryness to afford compound 6 (6.8 g) in 86% yield. ¹H NMR (400 MHz, CDCl₃) δ 9.75 (s, 1H), 7.27 (d, J=8.3 Hz, 1H), 6.29 (d, J=11.5 Hz, 1H), 6.12 (s, 2H), 2.17 (s, 3H); LCMS (ESI) m/z: 154 [M+H]⁺.

(S)-4-Ethyl-8-fluoro-4-hydroxy-9-methyl-1,12-dihydro-14H-pyrano[3′,4′:6,7]-indolizino[1,2-b]quinoline-3,14(4H)-dione 8. A mixture of compound 6 (6.0 g), compound 7 (10 g), and p-TsOH (1.4 g) in toluene (120 mL) was heated to 110±5° C. under N2 overnight. After the mixture was cooled to 20±5° C., MeOH (120 mL) was added. The mixture was stirred at 5±5° C. for 2 h, and filtered to yield a sticky solid, which was poured into MeOH (120 mL) and water (60 mL). The mixture was then heated to 70±5° C. for 2 h, cooled to 5±5° C., stirred at that temperature for additional 2 h, and filtered to yield a solid, which was dried at 60±5° C. to afford compound 8 (7.3 g) in 50% yield. ¹H NMR (600 MHz, DMSO-d₆) δ 8.58 (s, 1H), 8.02 (d, J=8.3 Hz, 1H), 7.84 (d, J=10.9 Hz, 1H), 7.30 (s, 1H), 6.52 (s, 10H), 5.41 (s, 2H), 5.21 (s, 2H), 2.46 (d, J=1.4 Hz, 3H), 1.87 (m, 2H), 0.88 (t, J=7.3 Hz, 4H); LCMS (ESI) m/z: 381 [M+H]⁺.

(S)-4-Ethyl-8-fluoro-4-hydroxy-11-(hydroxymethyl)-9-methyl-1,12-dihydro-14H-pyrano[3′,4′:6,7]indolizino[1,2-b]quinoline-3,14(4H)-dione 9. To a mixture of compound 8 (7.3 g) in MeOH (220 mL) and water (182 mL) at 5±5° C. was added sulfuric acid (98%, 73 mL) dropwise while keeping the temperature below 40° C. After the mixture was stirred at 5±5° C., ferrous sulfate heptahydrate (5.5 g) was added, followed by addition of 30% hydrogen peroxide solution (37 g) dropwise at 5±5° C. The mixture was stirred at room temperature overnight and cooled to 5±5° C. The pH of the mixture was adjusted to about 1 by addition of 3N KOH carefully while keeping the temperature below 40° C. After stirred at 5±5° C. for 2 h, the mixture was filtered and washed with water. The solid collected was dried at 60±5° C. to yield a crude product, which was purified with a silica gel column eluting with MeOH in DCM from 40:1 to 10:1 to afford compound 9 (3.07 g) in 39% yield. ¹H NMR (600 MHz, DMSO-d₆) δ 8.14 (d, J=8.0 Hz, 1H), 7.88 (d, J=10.6 Hz, 1H), 7.32 (s, 1H), 6.51 (s, 10H), 5.43 (s, 2H), 5.39 (s, 2H), 5.25 (s, 2H), 1.87 (m, 2H), 0.88 (t, J=7.4 Hz, 3H); LCMS (ESI) m/z: 411 [M+H]⁺.

(S)-4-Ethyl-8-fluoro-4-hydroxy-9-methyl-3,14-dioxo-3,4,12,14-tetrahydro-1H-pyrano[3′,4′:6,7]indolizino[1,2-b]quinoline-11-carbaldehyde 10. A mixture of compound 9 (1.08 g) in HOAc (180 mL) was stirred at 110±5° C. overnight in the presence of air. The mixture was cooled and evaporated to dryness to afford compound 10 (1.12 g). ¹H NMR (600 MHz, CDCl₃) δ 11.00 (s, 1H), 8.90 (d, J=7.7 Hz, 1H), 7.95 (d, J=10.3 Hz, 1H), 7.31 (s, 1H), 6.56 (s, 10H), 5.44 (s, 2H), 5.42 (s, 2H), 2.48 (s, 3H), 1.88 (m, 2H), 0.89 (t, J=7.4 Hz, 8H).

(S,E/Z)—4-Ethyl-8-fluoro-4-hydroxy-9-methyl-3,14-dioxo-3,4,12,14-tetrahydro-1H-pyrano[3′,4′:6,7]indolizino[1,2-b]quinoline-11-carbaldehyde O-(3-aminopropyl) oxime 12. A mixture of compound 10 (300 mg) and compound 11 (330 mg) in EtOH (9 mL) and pyridine (1.5 mL) was stirred at 85±5° C. for 1 h under N2. The mixture was then cooled to room temperature and evaporated to dryness to afford a crude product, which was purified with a silica gel column eluting with MeOH in DCM from 40:1 to 10:1 twice to afford compound 12 (160 mg) as a mixture of E/Z isomers. LCMS (ESI) m/z: 481.2 [M+H]⁺.

N—((S,E/Z)—17-Benzyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-8,13,16,19,22-pentaoxo-3,10-dioxa-2,7,12,15,18,21-hexaazatricos-1-en-23-yl)-6-(2,5-dioxo-2,5-dihydro-1H-pyrrol-1-yl)hexanamide A2. To a mixture of compound 12 (120 mg) and compound 13 (138 mg) in DMF (12 mL) stirred at 5±5° C. were added DMTMM (99 mg) in one portion and DIPEA (65 mg). After stirred at 20±5° C. for 2 h, the mixture was evaporated to dryness to yield a crude product, which was purified with prep-HPLC to afford compound A2 (28 mg) as a mixture of E/Z isomers. LCMS (ESI) m/z: 1079 [M+H]⁺.

N—((S,E/Z)—16-Benzyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-7,12,15,18,21-pentaoxo-3,9-dioxa-2,6,11,14,17,20-hexaazadocos-1-en-22-yl)-6-(2,5-dioxo-2,5-dihydro-1H-pyrrol-1-yl)hexanamide A1 was prepared as a mixture of E/Z isomers according to the synthetic procedures or methodologies exemplified herein. LCMS (ESI) m/z: 1093 [M+H]⁺.

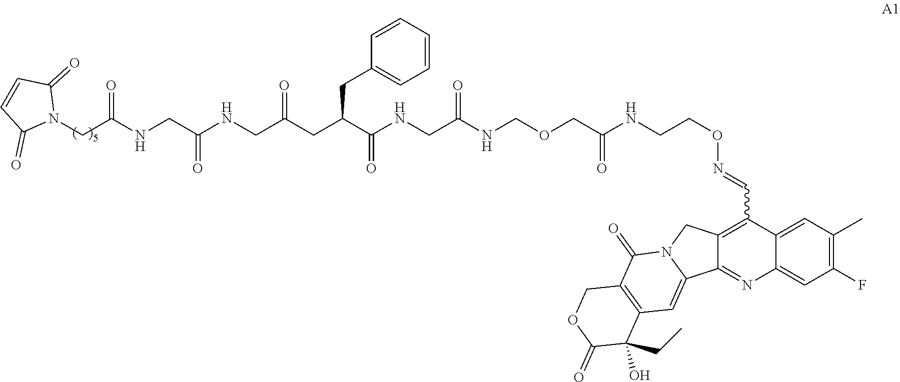

N-((17S,E/Z)—17-Benzyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-5-methyl-8,13,16,19,22-pentaoxo-3,10-dioxa-2,7,12,15,18,21-hexaazatricos-1-en-23-yl)-6-(2,5-dioxo-2,5-dihydro-1H-pyrrol-1-yl)hexanamide A3 is prepared similarly according to the synthetic procedures or methodologies exemplified herein.

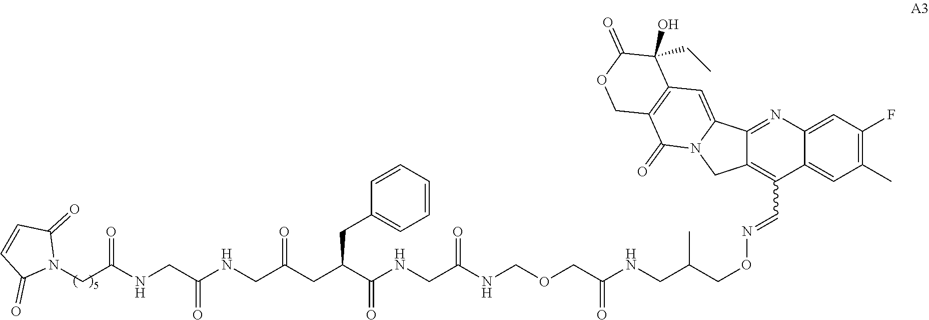

### Example 2

### Synthesis of (S,E/Z)—N-(3-((((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methylene)amino)oxy)propyl)-2-hydroxyacetamide B2

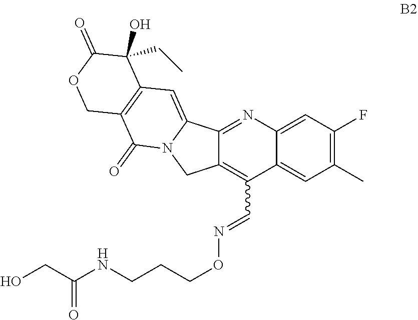 [SMILES: CC[C@@]1(O)C(=O)OCc2c1cc1n(c2=O)Cc2c-1nc1cc(F)c(C)cc1c2C=NOCCCNC(=O)CO]

Compound B2 was synthesized as shown in Scheme 2.

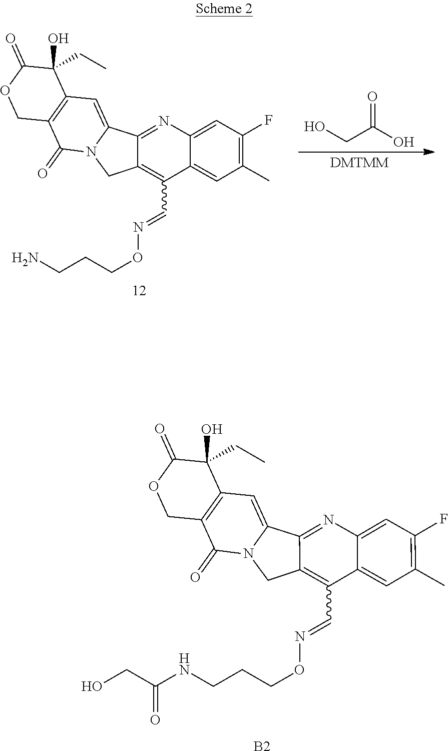

A mixture of compound 12 (80 mg), glycolic acid (12 mg), and DIPEA (45 mg) in DMF (8 mL) under N2 at −5±5° C. was added DMTMM (70 mg) in water (0.8 mL) dropwise. After stirred at 20±5° C. for 2 h, the mixture was evaporated to dryness to yield a crude product, which was purified with prep-HPLC to afford compound B2 (9 mg) as a mixture of E/Z isomers in 5 to 1 ratio. ¹HNMR (400 MHz, DMSO-d₆) for the E isomer: δ 9.17 (s, 1H), 8.29 (d, J=7.9 Hz, 1H), 7.73 (d, J=10.6 Hz, 1H), 7.58 (s, 1H), 5.55 (d, J=16.3 Hz, 1H), 5.35 (d, J=16.3 Hz, 1H), 5.32 (s, 2H), 4.45 (t, J=6.2 Hz, 2H), 3.94 (s, 2H), 3.44 (t, J=6.8 Hz, 2H), 2.49 (s, 3H), 2.06 (m, 2H), 1.91 (m, 2H), 0.97 (t, J=7.3 Hz, 3H); LCMS (ESI) m/z: 539.2 [M+H]⁺.

Compounds B1 and B3 were synthesized prepared similarly according to the synthetic procedures or methodologies exemplified herein.

(S,E/Z)—N-(2-((((4-Ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methylene)amino)oxy)ethyl)-2-hydroxyacetamide B1. ¹HNMR (400 MHz, DMSO-d₆) for the E isomer: δ 9.22 (s, 1H), 8.33 (d, J=7.9 Hz, 1H), 7.78 (d, J=10.5 Hz, 2H), 7.62 (s, 2H), 5.54 (d, J=5.2 Hz, 1H), 5.38 (d, J=3.4 Hz, 1H), 4.49 (t, J=5.4 Hz, 2H), 3.93 (s, 2H), 3.70 (t, J=5.4 Hz, 2H), 2.52 (s, 5H), 1.96-1.89 (m, 2H), 0.97 (t, J=7.4 Hz, 3H); LCMS (ESI) m/z: 525.3 [M+H]⁺.

N-(3-((((E/Z)—((S)-4-Ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methylene)amino)oxy)-2-methyl-propyl)-2-hydroxyacetamide B3. ¹HNMR (400 MHz, DMSO-d₆) for the E isomer: δ 9.14 (s, 1H), 8.23 (d, J=7.9 Hz, 1H), 7.67 (d, J=10.4 Hz, 1H), 7.55 (s, 1H), 5.56 (d, J=16.4 Hz, 1H), 5.36 (d, J=16.3 Hz, 1H), 5.23 (s, 2H), 4.34 (d, J=6.2 Hz, 2H), 3.99 (s, 2H), 2.32 (m, 1H), 1.95 (m, 2H), 1.08 (d, J=7.0 Hz, 3H), 1.00 (t, J=7.3 Hz, 3H); LCMS (ESI) m/z: 553.2 [M+H]⁺.

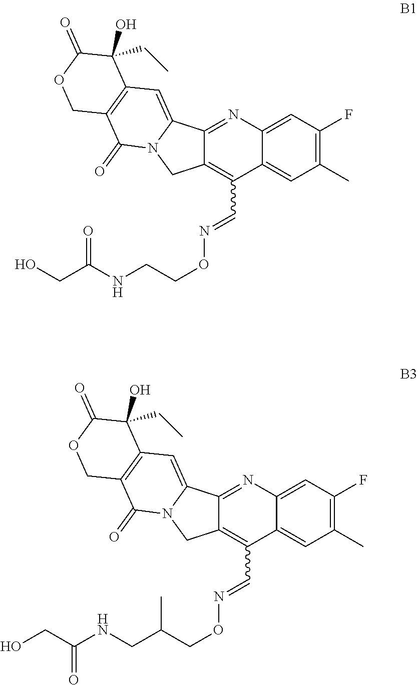 [SMILES: CC[C@@]1(O)C(=O)OCc2c1cc1n(c2=O)Cc2c-1nc1cc(F)c(C)cc1c2C=NOCCNC(=O)CO ; CC[C@@]1(O)C(=O)OCc2c1cc1n(c2=O)Cc2c-1nc1cc(F)c(C)cc1c2C=NOCC(C)CNC(=O)CO]

### Example 3

### Antibody-Drug Conjugate Preparation

Monoclonal antibody trastuzumab was buffer exchanged into a 20 mM sodium phosphate buffered saline solution and further diluted to 2.0 mg/mL in the same buffer. Ten molar equivalences of tris(2-carboxyethyl)phosphine (TCEP) were added to reduce the interchain disulfide bonds of trastuzumab to generate 8 free cysteines. Twenty molar equivalences of a drug with a maleimido-linker (drug-linker), e.g., compound A1, A2, or A3, were added to the reduced trastuzumab solution and incubated for approximately 1 h at room temperature. Excess TCEP and the drug-linker were then removed via a desalting column. The conjugation of the reduced trastuzumab with drug-linker compound A1, A2, and A3 formed ADC compounds ADC-A1, ADC-A2, and ADC-A3, respectively.

### Example 4

### Cell Viability Assays

Cancerous cells (5,000/well) were seeded in a culture media in a 96-well flat-bottom plate. A compound was prepared by a 3-fold serial dilution and added into each cell solutions. The cells were incubated for 4 or 5 days at 37° C. under 5% CO₂. CELLCOUNTING-LITE was added to each well, mixed, and incubated for 10 min at room temperature. The solution in each cell was transferred to a 96-well white opaque plate for reading. The results are summarized in Tables 1 and 2.

TABLE 1

| | IC₅₀ (nM) | | | |
|---|---|---|---|---|
| Cell Line | Deruxtecan | B1 | B2 | B3 |
| T47D | 1.9 | 2.8 | 2.4 | 1.3 |
| MCF7 | 5.7 | 19 | 9.2 | 21 |
| Skov-3 | 9.8 | 9.8 | 11 | 3.4 |
| KB | 7.2 | 31 | 19 | 18 |
| N87 | 9.9 | 11 | 9.1 | 7 |
| ASPC-1 | 29 | 54 | 40 | 25 |
| H322 | 8.2 | 12 | 12 | 3.9 |
| H226 | 388 | 153 | 113 | 401 |
| A375 | 3.6 | 9.4 | 6.9 | 5.9 |
| HT29 | 13 | 28 | 16 | 13 |
| A431 | 7.7 | 18 | 9.8 | 10 |
| Hut78 | 9.9 | 11 | 9.1 | 7 |
| Ramos | 3.8 | 7.8 | 6.6 | 7.2 |
| Jurkat | 2.3 | 3 | 2.1 | 1.2 |
| RS4; 11 | 0.65 | 1.1 | 0.88 | 0.73 |
| MV4; 11 | 6.9 | 18 | 14 | 10 |
| Raji | 0.42 | 0.45 | 0.58 | 0.32 |

TABLE 2

| | IC₅₀ (nM) | | |
|---|---|---|---|
| Cell Line | ADC-A1 | ADC-A2 | ADC-A3 |
| OVCAR-3 | 32 | 45 | 40 |
| N87 | 5.2 | 3.8 | 5.2 |

### Example 5

### MM Xenograft Mouse Model (Plasmacytoma Model)

Cancerous cells (1×10⁷) in 100 μL BD MATRIGEL™ are subcutaneously implanted in the right flank of CB. 17 SCID mice. From around day 15 onwards, all tumors are measured thrice weekly, and the length and width of each mouse tumor are recorded to calculate tumor volume (volume=length×(width²)×0.5). When the mean tumor volume reaches ˜150 mm³, mice are randomized into dose groups intraperitoneally twice weekly for 2 weeks. All tumors are measured and individual mice are euthanized once their tumor reach a mean tumor measurement of 1.5 cm.

Sequences described herein are provided in the sequence table below.

SEQUENCE TABLE

| SEQ ID NO: | Description | Amino Acid Sequence |
|---|---|---|
| 1 | Anti-B7H3-1 CDR1 | INAMG |
| | (Kabat) | |
| 2 | Anti-B7H3-1 CDR2 | GLSSNGDITRQNYAFYVKG |
| | (Kabat) | |
| 3 | Anti-B7H3-1 CDR3 | MPPAST |
| | (Kabat) | |
| 4 | Anti-B7H3-1 Variable | QVQLVESGGGLVQPGGSLRLSCSASGSTSNINAMG |
| | | WYRQAPGKEREFVAGLSSNGDITRQNYAFYVKGR |
| | | FTISRDNSKNTLYLQMNSLRAEDTAVYYCNEMPPA |
| | | STWGQGTQVTVSS |
| 5 | Anti-B7H3-1 | QVQLVESGGGLVQPGGSLRLSCSASGSTSNINAMG |
| | | WYRQAPGKEREFVAGLSSNGDITRQNYAFYVKGR |
| | | FTISRDNSKNTLYLQMNSLRAEDTAVYYCNEMPPA |
| | | STWGQGTQVTVSSDKTHTCPPCPAPELLGGPSVFLF |
| | | PPKPKDTLMISRTPEVTCVVVDVSHEDPEVKFNWY |
| | | VDGVEVHNAKTKPREEQYNSTYRVVSVLTVLHQD |
| | | WLNGKEYKCKVSNKALPAPIEKTISKAKGQPREPQ |
| | | VYTLPPSREEMTKNQVSLTCLVKGFYPSDIAVEWE |
| | | SNGQPENNYKTTPPVLDSDGSFFLYSKLTVDKSRW |
| | | QQGNVFSCSVMHEALHNHYTQKSLSLSPGK |
| 6 | Anti-B7H3-2 CDR1 | INAMA |
| | (Kabat) | |
| 7 | Anti-B7H3-2 CDR2 | GVTSSGSIVRENYAFYVKG |
| | (Kabat) | |
| 8 | Anti-B7H3-2 CDR3 | IPPYST |
| | (Kabat) | |
| 9 | Anti-B7H3-2 Variable | EVQLVESGGGLVKPGGSLRLSCAASGSTSSINAMA |
| | | WYRQAPGKQREFVAGVTSSGSIVRENYAFYVKGR |
| | | FTISRDNAKNSLYLQMNSLRAEDTAVYYCNAIPPY |
| | | STWGQGTQVTVSS |
| 10 | Anti-B7H3-2 | EVQLVESGGGLVKPGGSLRLSCAASGSTSSINAMA |
| | | WYRQAPGKQREFVAGVTSSGSIVRENYAFYVKGR |
| | | FTISRDNAKNSLYLQMNSLRAEDTAVYYCNAIPPY |
| | | STWGQGTQVTVSSDKTHTCPPCPAPELLGGPSVFLF |
| | | PPKPKDTLMISRTPEVTCVVVDVSHEDPEVKFNWY |
| | | VDGVEVHNAKTKPREEQYNSTYRVVSVLTVLHQD |
| | | WLNGKEYKCKVSNKALPAPIEKTISKAKGQPREPQ |
| | | VYTLPPSREEMTKNQVSLTCLVKGFYPSDIAVEWE |
| | | SNGQPENNYKTTPPVLDSDGSFFLYSKLTVDKSRW |
| | | QQGNVFSCSVMHEALHNHYTQKSLSLSPGK |
| 11 | Anti-CD20 CDRL1 | RASSSVSYIH |
| | (Kabat) | |
| 12 | Anti-CD20 CDRL2 | ATSNLAS |
| | (Kabat) | |
| 13 | Anti-CD20 CDRL3 | QQWTSNPPT |
| | (Kabat) | |
| 14 | Anti-CD20 CDRH1 | SYNMH |
| | (Kabat) | |
| 15 | Anti-CD20 CDRH2 | AIYPGNGDTSYNQKFKG |
| | (Kabat) | |
| 16 | Anti-CD20 CDRH3 | STYYGGDWYFNV |
| | (Kabat) | |
| 17 | Anti-CD20 Light Chain | QIVLSQSPAILSASPGEKVTMTCRASSSVSYIHWFQ |
| | Variable Region | QKPGSSPKPWIYATSNLASGVPVRFSGSGSGTSYSL |
| | | TISRVEAEDAATYYCQQWTSNPPTFGGGTKLEIK |
| 18 | Anti-CD20 Heavy Chain | QVQLQQPGAELVKPGASVKMSCKASGYTFTSYNM |
| | Variable Region | HWVKQTPGRGLEWIGAIYPGNGDTSYNQKFKGKA |
| | | TLTADKSSSTAYMQLSSLTSEDSAVYYCARSTYYG |
| | | GDWYFNVWGAGTTVTVSA |
| 19 | Anti-CD20 Light Chain | QIVLSQSPAILSASPGEKVTMTCRASSSVSYIHWFQ |
| | | QKPGSSPKPWIYATSNLASGVPVRFSGSGSGTSYSL |
| | | TISRVEAEDAATYYCQQWTSNPPTFGGGTKLEIKR |
| | | TVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| 20 | Anti-CD20 Heavy Chain | QVQLQQPGAELVKPGASVKMSCKASGYTFTSYNM |
| | | HWVKQTPGRGLEWIGAIYPGNGDTSYNQKFKGKA |
| | | TLTADKSSSTAYMQLSSLTSEDSAVYYCARSTYYG |
| | | GDWYFNVWGAGTTVTVSAASTKGPSVFPLAPSSK |
| | | STSGGTAALGCLVKDYFPEPVTVSWNSGALTSGVH |
| | | TFPAVLQSSGLYSLSSVVTVPSSSLGTQTYICNVNH |
| | | KPSNTKVDKKAEPKSCDKTHTCPPCPAPELLGGPS |
| | | VFLFPPKPKDTLMISRTPEVTCVVVDVSHEDPEVKF |
| | | NWYVDGVEVHNAKTKPREEQYNSTYRVVSVLTVL |
| | | HQDWLNGKEYKCKVSNKALPAPIEKTISKAKGQPR |
| | | EPQVYTLPPSREEMTKNQVSLTCLVKGFYPSDIAVE |
| | | WESNGQPENNYKTTPPVLDSDGSFFLYSKLTVDKS |
| | | RWQQGNVFSCSVMHEALHNHYTQKSLSLSPGK |
| 21 | Anti-FOLR1 CDRL1 | RASQSVSFAGTSLMH |
| | (Kabat) | |
| 22 | Anti-FOLR1 CDRL2 | RASNLEA |
| | (Kabat) | |
| 23 | Anti-FOLR1 CDRL3 | QQSREYPYT |
| | (Kabat) | |
| 24 | Anti-FOLR1 CDRH1 | GYFMN |
| | (Kabat) | |
| 25 | Anti-FOLR1 CDRH2 | RIHPYDGDTFYAQKFQG |
| | (Kabat) | |
| 26 | Anti-FOLR1 CDRH3 | YDGSRAMDY |
| | (Kabat) | |
| 27 | Anti-FOLR1 Light Chain | DIVLTQSPASLAVSPGQRATITCRASQSVSFAGTSL |
| | Variable Region | MHWYQQKPGQPPKLLIYRASNLEAGVPARFSGSGS |
| | | GTDFTLTINPVEANDAANYYCQQSREYPYTFGGGT |
| | | KLEIK |
| 28 | Anti-FOLR1 Heavy Chain | QVQLVQSGAEVKKPGASVKVSCKASGYTFTGYFM |
| | Variable Region | NWVRQAPGQGLEWIGRIHPYDGDTFYAQKFQGRV |
| | | TMTRDTSTSTVYMELSSLRSEDTAVYYCTRYDGSR |
| | | AMDYWGQGTTVTVSS |
| 29 | Anti-FOLR1 Light Chain | DIVLTQSPASLAVSPGQRATITCRASQSVSFAGTSL |
| | | MHWYQQKPGQPPKLLIYRASNLEAGVPARFSGSGS |
| | | GTDFTLTINPVEANDAANYYCQQSREYPYTFGGGT |
| | | KLEIKRTVAAPSVFIFPPSDEQLKSGTASVVCLLNNF |
| | | YPREAKVQWKVDNALQSGNSQESVTEQDSKDSTY |
| | | SLSSTLTLSKADYEKHKVYACEVTHQGLSSPVTKS |
| | | FNRGEC |
| 30 | Anti-FOLR1 Heavy Chain | QVQLVQSGAEVKKPGASVKVSCKASGYTFTGYFM |
| | | NWVRQAPGQGLEWIGRIHPYDGDTFYAQKFQGRV |
| | | TMTRDTSTSTVYMELSSLRSEDTAVYYCTRYDGSR |
| | | AMDYWGQGTTVTVSSASTKGPSVFPLAPSSKSTSG |
| | | GTAALGCLVKDYFPEPVTVSWNSGALTSGVHTFPA |
| | | VLQSSGLYSLSSVVTVPSSSLGTQTYICNVNHKPSN |
| | | TKVDKKVEPKSCDKTHTCPPCPAPELLGGPSVFLFP |
| | | PKPKDTLMISRTPEVTCVVVDVSHEDPEVKFNWYV |
| | | DGVEVHNAKTKPREEQYNSTYRVVSVLTVLHQDW |
| | | LNGKEYKCKVSNKALPAPIEKTISKAKGQPREPQV |
| | | YTLPPSREEMTKNQVSLTCLVKGFYPSDIAVEWES |
| | | NGQPENNYKTTPPVLDSDGSFFLYSKLTVDKSRWQ |
| | | QGNVFSCSVMHEALHNHYTQKSLSLSPGK |
| 31 | Anti-HER2 CDRL1 | RASQDVNTAVA |
| | (Kabat) | |
| 32 | Anti-HER2 CDRL2 | SASFLYS |
| | (Kabat) | |
| 33 | Anti-HER2 CDRL3 | QQHYTTPPT |
| | (Kabat) | |
| 34 | Anti-HER2 CDRH1 | DTYIH |
| | (Kabat) | |
| 35 | Anti-HER2 CDRH2 | RIYPTNGYTRYADSVKG |
| | (Kabat) | |
| 36 | Anti-HER2 CDRH3 | WGGDGFYAMDY |
| | (Kabat) | |
| 37 | Anti-HER2 Light Chain | DIQMTQSPSSLSASVGDRVTITCRASQDVNTAVAW |
| | Variable Region | YQQKPGKAPKLLIYSASFLYSGVPSRFSGSRSGTDF |
| | | TLTISSLQPEDFATYYCQQHYTTPPTFGQGTKVEIK |
| 38 | Anti-HER2 Heavy Chain | EVQLVESGGGLVQPGGSLRLSCAASGFNIKDTYIH |
| | Variable Region | WVRQAPGKGLEWVARIYPTNGYTRYADSVKGRFT |
| | | ISADTSKNTAYLQMNSLRAEDTAVYYCSRWGGDG |
| | | FYAMDYWGQGTLVTVSS |
| 39 | Anti-HER2 Light Chain | DIQMTQSPSSLSASVGDRVTITCRASQDVNTAVAW |
| | | YQQKPGKAPKLLIYSASFLYSGVPSRFSGSRSGTDF |
| | | TLTISSLQPEDFATYYCQQHYTTPPTFGQGTKVEIK |
| | | RTVAAPSVFIFPPSDEQLKSGTASVVCLLNNFYPRE |
| | | AKVQWKVDNALQSGNSQESVTEQDSKDSTYSLSS |
| | | TLTLSKADYEKHKVYACEVTHQGLSSPVTKSFNRG |
| | | EC |
| 40 | Anti-HER2 Heavy Chain | EVQLVESGGGLVQPGGSLRLSCAASGFNIKDTYIH |
| | | WVRQAPGKGLEWVARIYPTNGYTRYADSVKGRFT |
| | | ISADTSKNTAYLQMNSLRAEDTAVYYCSRWGGDG |
| | | FYAMDYWGQGTLVTVSSASTKGPSVFPLAPSSKST |
| | | SGGTAALGCLVKDYFPEPVTVSWNSGALTSGVHTF |
| | | PAVLQSSGLYSLSSVVTVPSSSLGTQTYICNVNHKP |
| | | SNTKVDKKVEPKSCDKTHTCPPCPAPELLGGPSVFL |
| | | FPPKPKDTLMISRTPEVTCVVVDVSHEDPEVKFNW |
| | | YVDGVEVHNAKTKPREEQYNSTYRVVSVLTVLHQ |
| | | DWLNGKEYKCKVSNKALPAPIEKTISKAKGQPREP |
| | | QVYTLPPSREEMTKNQVSLTCLVKGFYPSDIAVEW |
| | | ESNGQPENNYKTTPPVLDSDGSFFLYSKLTVDKSR |
| | | WQQGNVFSCSVMHEALHNHYTQKSLSLSPGK |
| 41 | Anti-MSLN-1 CDR1 | VNAYG |
| | (Kabat) | |
| 42 | Anti-MSLN-1 CDR2 | IISAGGTTNYADSVKG |
| | (Kabat) | |
| 43 | Anti-MSLN-1 CDR3 | QRRIGMLRDY |
| | (Kabat) | |
| 44 | Anti-MSLN-1 Variable | QVQLVESGGGLVQPGGSLRLSCAASGITFPVNAYG |
| | | WYRQAPGKQRDLVAIISAGGTTNYADSVKGRFTIS |
| | | RDNSKNTLYLQMNSLRAEDTAVYYCYLQRRIGML |
| | | RDYWGQGTQVTVSS |
| 45 | Anti-MSLN-1 | QVQLVESGGGLVQPGGSLRLSCAASGITFPVNAYG |
| | | WYRQAPGKQRDLVAIISAGGTTNYADSVKGRFTIS |
| | | RDNSKNTLYLQMNSLRAEDTAVYYCYLQRRIGML |
| | | RDYWGQGTQVTVSSDKTHTCPPCPAPELLGGPSVF |
| | | LFPPKPKDTLMISRTPEVTCVVVDVSHEDPEVKFN |
| | | WYVDGVEVHNAKTKPREEQYNSTYRVVSVLTVLH |
| | | QDWLNGKEYKCKVSNKALPAPIEKTISKAKGQPRE |
| | | PQVYTLPPSREEMTKNQVSLTCLVKGFYPSDIAVE |
| | | WESNGQPENNYKTTPPVLDSDGSFFLYSKLTVDKS |
| | | RWQQGNVFSCSVMHEALHNHYTQKSLSLSPGK |
| 46 | Anti-MSLN-2 CDR1 | SYVMA |
| | (Kabat) | |
| 47 | Anti-MSLN-2 CDR2 | SINWSSGRLIYADSVKG |
| | (Kabat) | |
| | Anti-MSLN-2 CDR3 | GRY |
| | (Kabat) | |
| 48 | Anti-MSLN-2 Variable | QVQLVESGGGLVQPGGSLRLSCAASGRTLESYVM |
| | | AWFRQAPGKEREAVASINWSSGRLIYADSVKGRFT |
| | | ISRDNSKNTLYLQMNSLRAEDTAVYYCAAGRYWG |
| | | QGTQVTVSS |
| 49 | Anti-MSLN-2 | QVQLVESGGGLVQPGGSLRLSCAASGRTLESYVM |
| | | AWFRQAPGKEREAVASINWSSGRLIYADSVKGRFT |
| | | ISRDNSKNTLYLQMNSLRAEDTAVYYCAAGRYWG |
| | | QGTQVTVSSDKTHTCPPCPAPELLGGPSVFLFPPKP |
| | | KDTLMISRTPEVTCVVVDVSHEDPEVKFNWYVDG |
| | | VEVHNAKTKPREEQYNSTYRVVSVLTVLHQDWLN |
| | | GKEYKCKVSNKALPAPIEKTISKAKGQPREPQVYT |
| | | LPPSREEMTKNQVSLTCLVKGFYPSDIAVEWESNG |
| | | QPENNYKTTPPVLDSDGSFFLYSKLTVDKSRWQQG |
| | | NVFSCSVMHEALHNHYTQKSLSLSPGK |
| 50 | Anti-TROP2 CDRL1 | KASQDVSIAVA |
| | (Kabat) | |
| 51 | Anti-TROP2 CDRL2 | SASYRYT |
| | (Kabat) | |
| 52 | Anti-TROP2 CDRL3 | QQHYITPLT |
| | (Kabat) | |
| 53 | Anti-TROP2 CDRH1 | NYGMN |
| | (Kabat) | |
| 54 | Anti-TROP2 CDRH2 | WINTYTGEPTYTDDFKG |
| | (Kabat) | |
| 55 | Anti-TROP2 CDRH3 | GGFGSSYWYFDV |
| | (Kabat) | |
| 56 | Anti-TROP2 Light Chain | DIQLTQSPSSLSASVGDRVSITCKASQDVSIAVAWY |
| | Variable Region | QQKPGKAPKLLIYSASYRYTGVPDRFSGSGSGTDFT |
| | | LTISSLQPEDFAVYYCQQHYITPLTFGAGTKVEIK |
| 57 | Anti-TROP2 Heavy Chain | QVQLQQSGSELKKPGASVKVSCKASGYTFTNYGM |
| | Variable Region | NWVKQAPGQGLKWMGWINTYTGEPTYTDDFKGR |
| | | FAFSLDTSVSTAYLQISSLKADDTAVYFCARGGFGS |
| | | SYWYFDVWGQGSLVTVSS |
| 58 | Anti-TROP2 Light Chain | DIQLTQSPSSLSASVGDRVSITCKASQDVSIAVAWY |
| | | QQKPGKAPKLLIYSASYRYTGVPDRFSGSGSGTDFT |
| | | LTISSLQPEDFAVYYCQQHYITPLTFGAGTKVEIKR |
| | | TVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| 59 | Anti-TROP2 Heavy Chain | QVQLQQSGSELKKPGASVKVSCKASGYTFTNYGM |
| | | NWVKQAPGQGLKWMGWINTYTGEPTYTDDFKGR |
| | | FAFSLDTSVSTAYLQISSLKADDTAVYFCARGGFGS |
| | | SYWYFDVWGQGSLVTVSSASTKGPSVFPLAPSSKS |
| | | TSGGTAALGCLVKDYFPEPVTVSWNSGALTSGVHT |
| | | FPAVLQSSGLYSLSSVVTVPSSSLGTQTYICNVNHK |
| | | PSNTKVDKRVEPKSCDKTHTCPPCPAPELLGGPSVF |
| | | LFPPKPKDTLMISRTPEVTCVVVDVSHEDPEVKFN |
| | | WYVDGVEVHNAKTKPREEQYNSTYRVVSVLTVLH |
| | | QDWLNGKEYKCKVSNKALPAPIEKTISKAKGQPRE |
| | | PQVYTLPPSREEMTKNQVSLTCLVKGFYPSDIAVE |
| | | WESNGQPENNYKTTPPVLDSDGSFFLYSKLTVDKS |
| | | RWQQGNVFSCSVMHEALHNHYTQKSLSLSPGK |

The examples set forth above are provided to give those of ordinary skill in the art with a complete disclosure and description of how to make and use the claimed embodiments and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A compound of Formula (II): 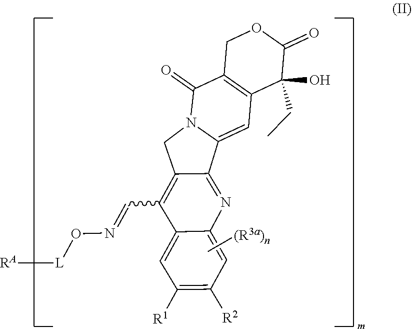
or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
R^{A} is an antibody or an antigen-binding fragment thereof;
R¹ and R² are each independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R^{3a} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
each L is independently a linker;
m is an integer of 1, 2, 3, 4, 5, 6, 7, 8, 9, 10, 11, 12, 13, 14, 15, or 16; and
n is an integer of 0, 1, 2, 3, 4, 5, 6, 7, or 8;
wherein each alkyl, heteroalkyl, alkenyl, alkynyl, cycloalkyl, aryl, aralkyl, heteroaryl, and heterocyclyl is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e} C(O)NR^{f}R^{g}, —NR^{e}C(O)SR^{f}, —NR^{e} C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

2. The compound of claim 1, wherein each n is independently an integer of 0, 1, or 2.

3. The compound of claim 1 or 2, having the structure of Formula (III): 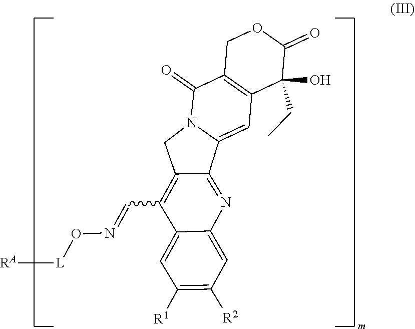
or a diastereomer, a mixture of two or more diastereomers, a tautomer, a mixture of two or more tautomers, or an isotopic variant thereof; or a pharmaceutically acceptable salt, solvate, hydrate, or prodrug thereof.

4. The compound of any one of claims 1 to 3, having the structure of Formula (IV): 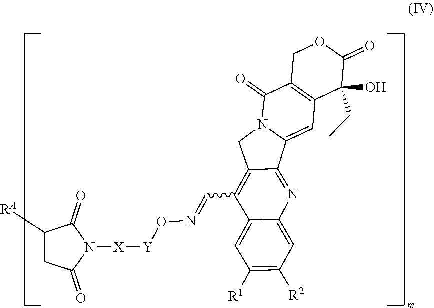
or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
each X is independently C₁₋₄₀ alkylene, C₁₋₄₀ heteroalkylene, C₂₋₄₀ alkenylene, C₂₋₄₀ heteroalkenylene, C₂₋₄₀ alkynylene, or C₂₋₄₀ heteroalkynylene, wherein one or more methylene groups are each independently and optionally replaced by a divalent group and each divalent group is independently C₃₋₁₀ cycloalkylene, C₆₋₁₄ arylene, heteroarylene, or heterocyclylene; and
each Y is independently a bond, C₁₋₆ alkylene, C₁₋₆ heteroalkylene, C₂₋₆ alkenylene, C₂₋₆ alkynylene, C₃₋₁₀ cycloalkylene, C₆₋₁₄ arylene, C₇₋₁₅ aralkylene, heteroarylene, or heterocyclylene;
wherein each alkylene, heteroalkylene, alkenylene, heteroalkenylene, alkynylene, heteroalkynylene, cycloalkylene, arylene, heteroarylene, and heterocyclylene is optionally substituted with one or more substituents Q.

5. The compound of claim 4, wherein each X is independently C₁₋₄₀ heteroalkylene, optionally substituted with one or more substituents Q.

6. The compound of claim 4 or 5, wherein each X is

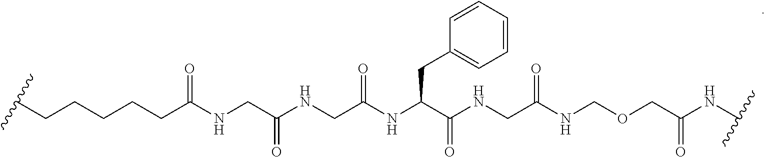 [SMILES: *CCCCCC(=O)NCC(=O)NCC(=O)N[C@@H](Cc1ccccc1)C(=O)NCC(=O)NCOCC(=O)N*]

7. The compound of any one of claims 4 to 6, wherein each Y is independently C₁₋₆ alkylene, optionally substituted with one or more substituents Q.

8. The compound of any one of claims 4 to 7, wherein each Y is independently —(CH₂)ᵣ— and r is an integer of 1, 2, 3, 4, 5, or 6.

9. The compound of any one of claims 4 to 8, wherein each Y is independently ethane-1,1-diyl, propane-1,3-diyl, or 2-methylpropane-1,3-diyl.

10. The compound of any one of claims 1 to 9, wherein each R¹ is independently C₁₋₆ alkyl, optionally substituted with one or more substituents Q.

11. The compound of any one of claims 1 to 10, wherein each R¹ is methyl.

12. The compound of any one of claims 1 to 11, wherein each R² is independently halo.

13. The compound of any one of claims 1 to 12, wherein each R² is fluoro.

14. The compound of any one of claims 1 to 13, wherein L is a cleavable linker.

15. The compound of any one of claims 1 to 13, wherein L is a non-cleavable linker.

16. The compound of any one of claims 1 to 15, wherein L is C₁₋₅₀ alkylene, C₁₋₅₀ heteroalkylene, C₂₋₅₀ alkenylene, C₂₋₅₀ heteroalkenylene, C₂₋₅₀ alkynylene, or C₂₋₅₀ heteroalkynylene, wherein one or more methylene groups are each independently and optionally replaced by a divalent group and each divalent group is independently C₃₋₁₀ cycloalkylene, C₆₋₁₂ arylene, heteroarylene, or heterocyclylene; and wherein the alkylene, heteroalkylene, alkenylene, heteroalkenylene, alkynylene, heteroalkynylene, cycloalkylene, arylene, heteroarylene, and heterocyclylene are each optionally substituted with one or more substituents Q.

17. The compound of any one of claims 1 to 16, wherein L is C₁₋₅₀ alkylene or C₅₀ heteroalkylene, wherein one or more methylene groups are each independently and optionally replaced by a divalent group and each divalent group is independently C₃₋₁₀ cycloalkylene, C₆₋₁₀ arylene, heteroarylene, or heterocyclylene; and wherein the alkylene, heteroalkylene, alkenylene, heteroalkenylene, alkynylene, heteroalkynylene, cycloalkylene, arylene, heteroarylene, and heterocyclylene are each optionally substituted with one or more substituents Q.

18. The compound of any one of claims 1 to 17, wherein L is C₁₋₅₀ alkylene or C₁₋₅₀ heteroalkylene, wherein one, two, or three methylene groups are each independently and optionally replaced by a divalent group, and each divalent group is independently cyclohexane-1,4-diyl, phen-1,3-diyl, phen-1,4-diyl, triazol-1,4-diyl, or 2,5-dioxopyrrolidin-1,3-diyl.

19. The compound of any one of claims 1 to 18, wherein L is:

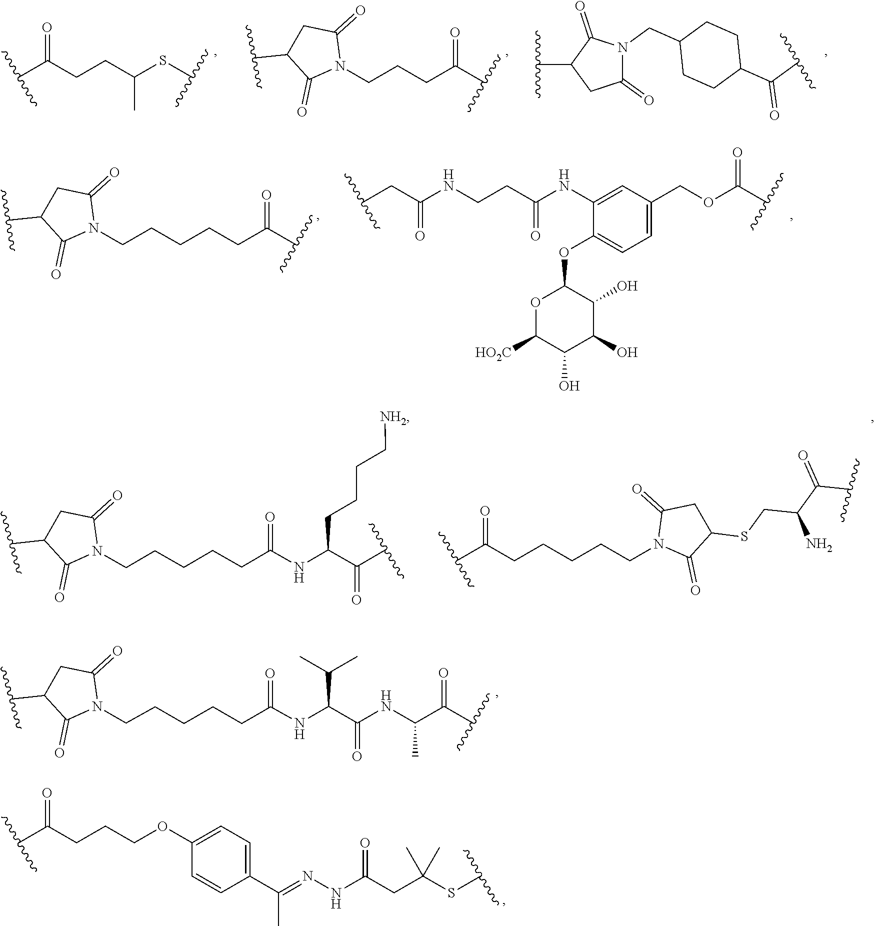

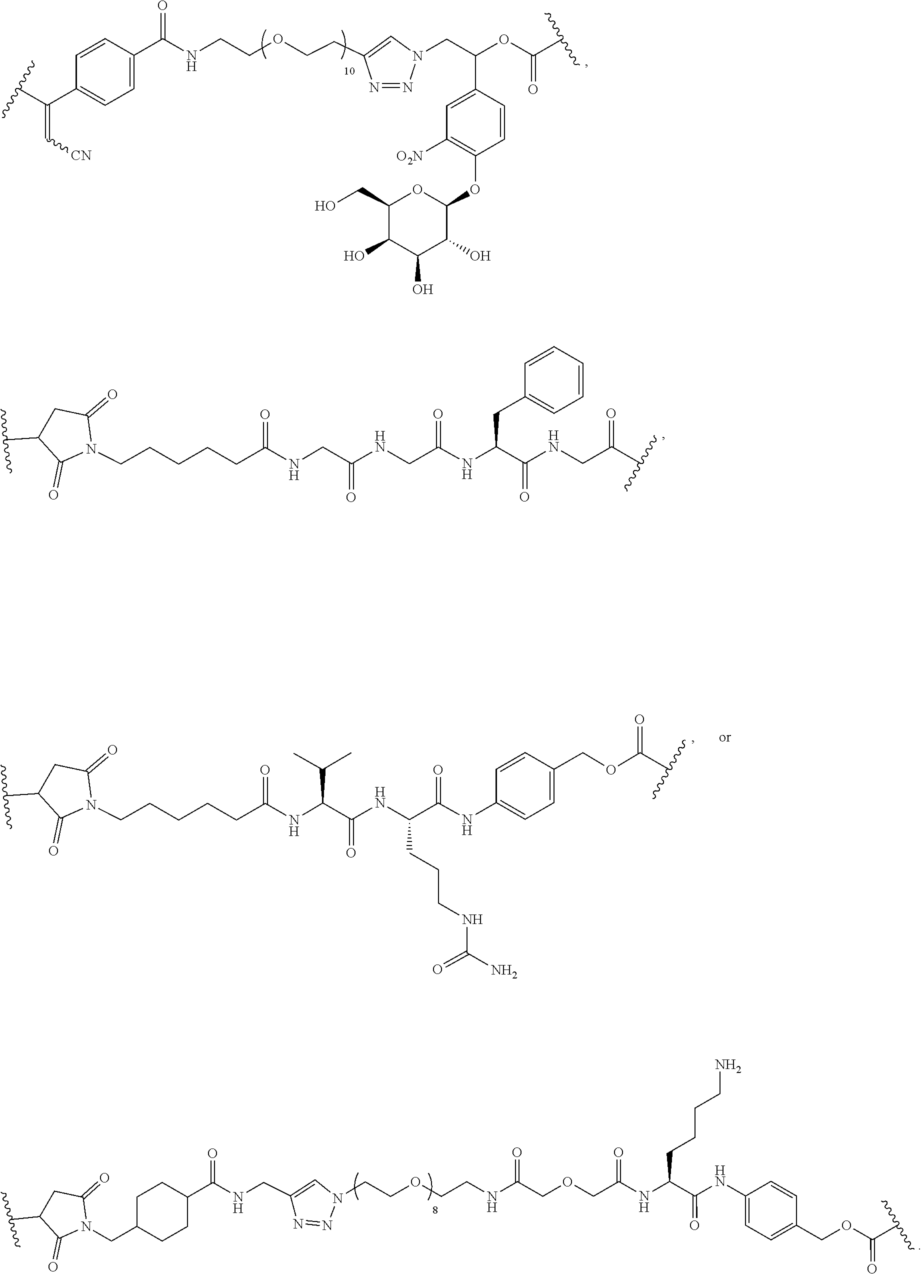

20. The compound of any one of claims 1 to 18, wherein L is:

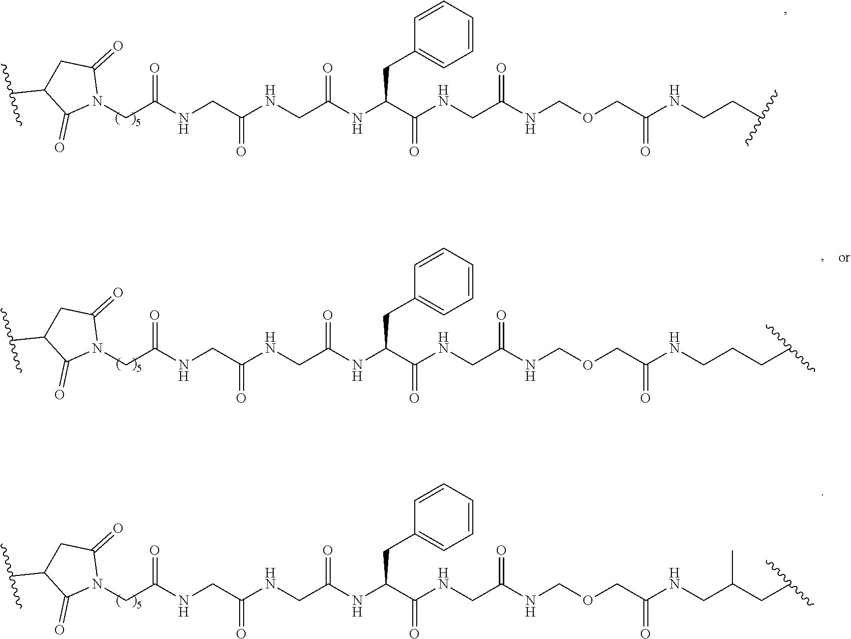

21. The compound of claim 1, wherein the compound is: 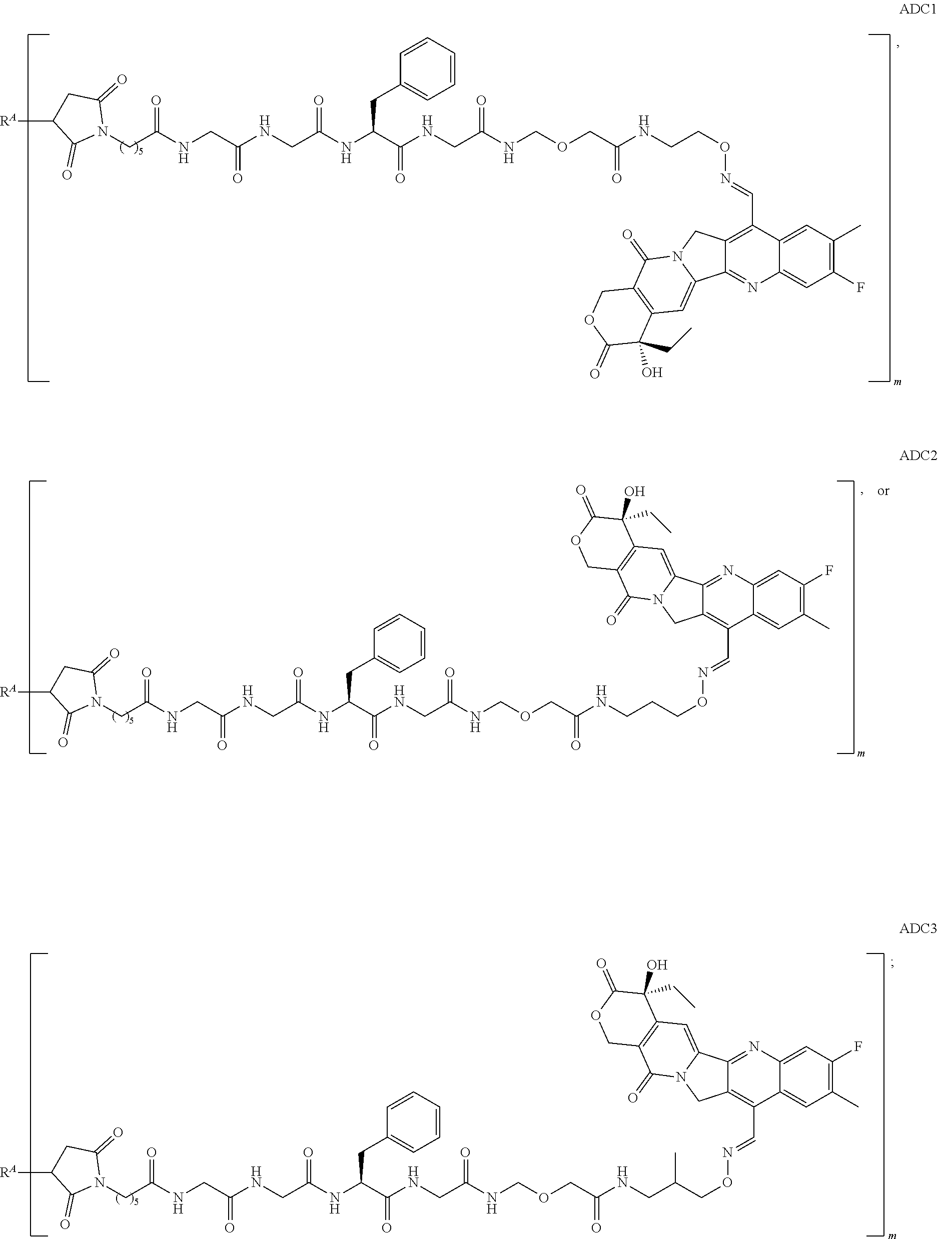
or a diastereomer, a mixture of two or more diastereomers, a tautomer, a mixture of two or more tautomers, or an isotopic variant thereof; or a pharmaceutically acceptable salt, solvate, hydrate, or prodrug thereof.

22. The compound of any one of claims 1 to 21, wherein R^{A} is a monoclonal antibody or an antigen-binding fragment thereof.

23. The compound of any one of claims 1 to 22, wherein R^{A} is a human, humanized, or chimeric antibody, or an antigen-binding fragment thereof.

24. The compound of any one of claims 1 to 23, wherein R^{A} is an IgG1, IgG2, IgG3, or IgG4 antibody, or an antigen-binding fragment thereof.

25. The compound of any one of claims 1 to 24, wherein R^{A} is anti-B7H3 antibody, anti-CD20 antibody, anti-FOLR1 antibody, anti-HER2 antibody, anti-MSLN antibody, anti-TROP2 antibody, or an antigen-binding fragment thereof.

26. The compound of any one of claims 1 to 25, wherein R^{A} is an anti-B7H3 single domain antibody or an antigen-binding fragment thereof.

27. The compound of claim 26, wherein the anti-1B71-13 single domain antibody comprises: (i) a CDR1 of SEQ ID NO: 1, a CDR2 of SEQ ID NO: 2, and a CDR3 of SEQ ID NO: 3; or (ii) a CDR1 of SEQ ID NO: 6, a CDR2 of SEQ ID NO: 7, and a CDR3 of SEQ ID NO: 8.

28. The compound of any one of claims 1 to 25, wherein R^{A} is an anti-CD20 antibody or an antigen-binding fragment thereof.

29. The compound of claim 28, wherein the anti-CD20 antibody comprises a CDRL1 of SEQ ID NO: 11; a CDRL2 of SEQ ID NO: 12; a CDRL3) of SEQ ID NO: 13; a CDRH1 of SEQ ID NO: 14; a CDRH2 of SEQ ID NO: 15; and a CDRH3 of SEQ ID NO: 16.

30. The compound of any one of claims 1 to 25, wherein R^{A} is an anti-FOLR1 antibody or an antigen-binding fragment thereof.

31. The compound of claim 30, wherein the anti-FOLR1 antibody comprises a CDRL1 of SEQ ID NO: 21; a CDRL2 of SEQ ID NO: 22; a CDRL3 of SEQ ID NO: 23; a CDRH1 of SEQ ID NO: 24; a CDRH2 of SEQ ID NO: 25; and a CDRH3 of SEQ ID NO: 26.

32. The compound of any one of claims 1 to 25, wherein R^{A} is an anti-HER2 antibody or an antigen-binding fragment thereof.

33. The compound of claim 32, wherein the anti-HER2 antibody comprises a CDRL1 of SEQ ID NO: 31; a CDRL2 of SEQ ID NO: 32: a CDRL3 of SEQ ID NO: 33: a CDRH1 of SEQ ID NO: 34; a CDR1-2 of SEQ ID NO: 35; and a CDRH3 of SEQ ID NO: 36.

34. The compound of claim 32 or 33, wherein the anti-HER2 antibody comprises a light chain variable region of SEQ ID NO: 37 and a heavy chain variable region of SEQ ID NO: 38.

35. The compound of any one of claims 32 to 34, wherein the anti-HER2 antibody comprises a light chain of SEQ ID NO: 39 and a heavy chain of SEQ ID NO: 40.

36. The compound of any one of claims 1 to 25, wherein R^{A} is an anti-MSLN single domain antibody or an antigen-binding fragment thereof.

37. The compound of claim 36, wherein the anti-MSLN single domain antibody comprises: (i) a CDR1 of SEQ ID NO: 41, a CDR2 of SEQ ID NO: 42, and a CDR3 of SEQ ID NO: 43; or (ii) a CDR1 of SEQ ID NO: 46, a CDR2 of SEQ ID NO: 47, and a CDR3 of GRY.

38. The compound of any one of claims 1 to 25, wherein R^{A} is an anti-TROP2 antibody or an antigen-binding fragment thereof.

39. The compound of claim 38, wherein the anti-TROP2 antibody comprises a CDRL1 of SEQ ID NO: 50; a CDRL2 of SEQ ID NO: 51; a CDRL3 of SEQ ID NO: 52; a CDRH1 of SEQ ID NO: 53; a CDRH2 of SEQ ID NO: 54; and a CDRH3 of SEQ ID NO: 55.

40. The compound of any one of claims 1 to 39, wherein m is an integer of 1, 2, 3, 4, 5, 6, 7, 8, 9, 10, 11, or 12.

41. The compound of any one of claims 1 to 40, wherein m is an integer of 4, 5, 6, 7, or 8.

42. The compound of any one of claims 1 to 41, wherein m is an integer of 8.

43. The compound of claim 1, wherein the compound is a trastuzumab ADC selected from ADC-A1, ADC-A2, and ADC-A3.

44. A pharmaceutical composition comprising the compound of any one of claims 1 to 43, or a diastereomer, a mixture of two or more diastereomers, a tautomer, a mixture of two or more tautomers, or an isotopic variant thereof; or a pharmaceutically acceptable salt, solvate, or hydrate thereof; and a pharmaceutically acceptable excipient.

45. The pharmaceutical composition of claim 44, wherein the composition is in single dosage form.

46. The pharmaceutical composition of claim 44 or 45, wherein the composition is in a parenteral or intravenous dosage form.

47. The pharmaceutical composition of claim 46, wherein the composition is formulated in an intravenous dosage form.

48. A method of treating, preventing, or ameliorating one or more symptoms of a proliferative disease in a subject, comprising administering to the subject in need thereof a therapeutically effective amount of a compound of any one of claims 1 to 43 or a pharmaceutical composition of any one of claims 44 to 47.

49. The method of claim 48, wherein the proliferative disease is cancer.

50. The method of claim 48 or 49, wherein the cancer is relapsed or refractory.

51. The method of any one of claims 48 to 50, wherein the cancer is metastatic.

52. The method of any one of claims 48 to 51, wherein the cancer is drug-resistant.

53. The method of any one of claims 48 to 52, wherein the subject is a human.

54. A method of inhibiting the growth of a cell, comprising contacting the cell with an effective amount of a compound of any one of claims 1 to 43 or a pharmaceutical composition of any one of claims 44 to 47.

55. The method of claim 54, wherein the cell is a cancerous cell.

56. A compound of Formula (IA): 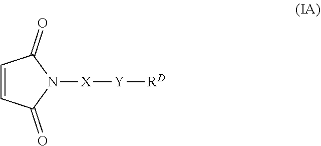
or a diastereomer, a mixture of two or more diastereomers, a tautomer, a mixture of two or more tautomers, or an isotopic variant thereof; or a pharmaceutically acceptable salt, solvate, or hydrate thereof; wherein:
X is C₁₋₄₀ alkylene, C₁₋₄₀ heteroalkylene, C₂₋₄₀ alkenylene, C₂₋₄₀ heteroalkenylene, C₂₋₄₀ alkynylene, or C₂₋₄₀ heteroalkynylene, wherein one or more methylene groups are each independently and optionally replaced by a divalent group and each divalent group is independently C₃₋₁₀ cycloalkylene, C₆₋₁₄ arylene, heteroarylene, or heterocyclylene;
Y is independently a bond, C₁₋₆ alkylene, C₁₋₆ heteroalkylene, C₂₋₆ alkenylene, C₂₋₆ alkynylene, C₃₋₁₀ cycloalkylene, C₆₋₁₄ arylene, C₇₋₁₅ aralkylene, heteroarylene, or heterocyclylene; and
R^{D} is 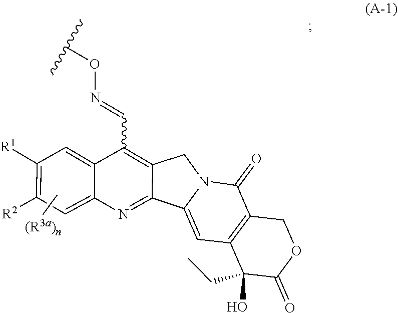
wherein:
R¹ and R² are each independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1C}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R^{3a} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c} or —S(O)₂NR^{1b}R^{1c};
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and
n is an integer of 0, 1, 2, 3, 4, 5, 6, 7, or 8;
wherein each alkyl, alkylene, heteroalkyl, heteroalkylene, alkenyl, alkenylene, heteroalkenylene, alkynyl, alkynylene, heteroalkynylene, cycloalkyl, cycloalkylene, aryl, arylene, aralkyl, heteroaryl, heteroarylene, heterocyclyl, and heterocyclylene is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{1b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

57. The compound of claim 56, having the structure of Formula (IIA): 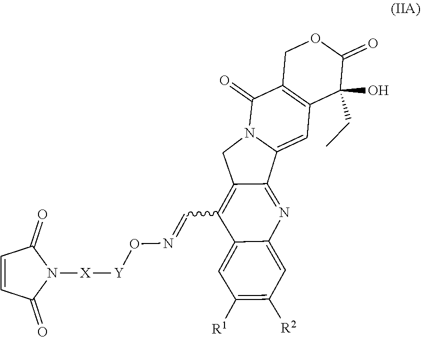
or a diastereomer, a mixture of two or more diastereomers, a tautomer, a mixture of two or more tautomers, or an isotopic variant thereof; or a pharmaceutically acceptable salt, solvate, or hydrate thereof.

58. The compound of claim 56 or 57, wherein each R¹ is independently C₁₋₆ alkyl, optionally substituted with one or more substituents Q.

59. The compound of any one of claims 56 to 58, wherein each R¹ is methyl.

60. The compound of any one of claims 56 to 59, wherein each R² is independently halo.

61. The compound of any one of claims 56 to 60, wherein each R² is fluoro.

62. The compound of any one of claims 56 to 61, wherein each X is independently C₁₋₄₀ heteroalkylene, optionally substituted with one or more substituents Q.

63. The compound of any one of claims 56 to 62, wherein each X is

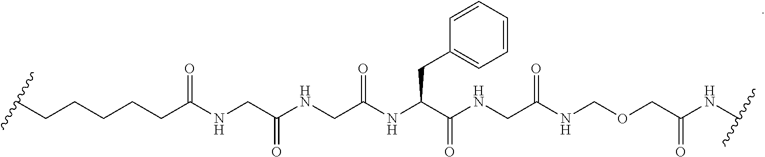 [SMILES: *CCCCCC(=O)NCC(=O)NCC(=O)N[C@@H](Cc1ccccc1)C(=O)NCC(=O)NCOCC(=O)N*]

64. The compound of any one of claims 56 to 63, wherein each Y is independently C₁₋₆ alkylene, optionally substituted with one or more substituents Q.

65. The compound of any one of claims 56 to 64, wherein each Y is independently —(CH₂)ᵣ— and r is an integer of 1, 2, 3, 4, 5, or 6.

66. The compound of any one of claims 56 to 65, wherein each Y is independently ethane-1,1-diyl, propane-1,3-diyl, or 2-methylpropane-1,3-diyl.

67. The compound of claim 56, wherein R^{D} is

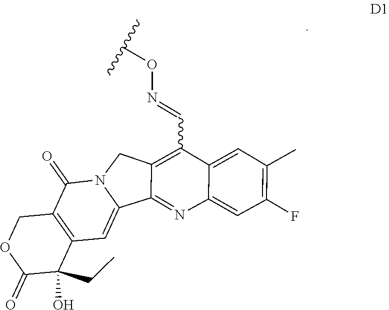 [SMILES: *ON=Cc1c2c(nc3cc(F)c(C)cc13)-c1cc3c(c(=O)n1C2)COC(=O)[C@]3(O)CC]

68. The compound of claim 56, wherein the compound is:
N—((S,E/Z)—16-benzyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-7,12,15,18,21-pentaoxo-3,9-dioxa-2,6,11,14,17,20-hexaazadocos-1-en-22-yl)-6-(2,5-dioxo-2,5-dihydro-1H-pyrrol-1-yl)hexanamide A1;
N—((S,E/Z)—17-benzyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-8,13,16,19,22-pentaoxo-3,10-dioxa-2,7,12,15,18,21-hexaazatricos-1-en-23-yl)-6-(2,5-dioxo-2,5-dihydro-1H-pyrrol-1-yl)hexanamide A2; or
N-((17S,E/Z)—17-benzyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-5-methyl-8,13,16,19,22-pentaoxo-3,10-dioxa-2,7,12,15,18,21-hexaazatricos-1-en-23-yl)-6-(2,5-dioxo-2,5-dihydro-1H-pyrrol-1-yl)hexanamide A3;
or a diastereomer, a mixture of two or more diastereomers, a tautomer, a mixture of two or more tautomers, or an isotopic variant thereof; or a pharmaceutically acceptable salt, solvate, or hydrate thereof.

69. A compound selected from:
(S,E/Z)—N-(2-((((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methylene)amino)oxy)ethyl)-2-hydroxyacetamide B1;
(S,E/Z)—N-(3-((((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methylene)amino)oxy)propyl)-2-hydroxyacetamide B2; or
N-(3-((((E/Z)—((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methylene)amino)oxy)-2-methyl-propyl)-2-hydroxyacetamide B3;
or a diastereomer, a mixture of two or more diastereomers, a tautomer, a mixture of two or more tautomers, or an isotopic variant thereof; or a pharmaceutically acceptable salt, solvate, or hydrate thereof.


SEQUENCE LISTING (59 sequences)

SEQ ID NO: 1 - protein, 5 aa, Artificial Sequence
  Synthetic Construct
INAMG

SEQ ID NO: 2 - protein, 19 aa, Artificial Sequence
  Synthetic Construct
GLSSNGDITRQNYAFYVKG

SEQ ID NO: 3 - protein, 6 aa, Artificial Sequence
  Synthetic Construct
MPPAST

SEQ ID NO: 4 - protein, 117 aa, Artificial Sequence
  Synthetic Construct
QVQLVESGGGLVQPGGSLRLSCSASGSTSNINAMGWYRQAPGKEREFVAGLSSNGDITRQNYAFYVKGRFTISRDNSKNTLYLQMNSLRAEDTAVYYCNEMPPASTWGQGTQVTVSS

SEQ ID NO: 5 - protein, 344 aa, Artificial Sequence
  Synthetic Construct
QVQLVESGGGLVQPGGSLRLSCSASGSTSNINAMGWYRQAPGKEREFVAGLSSNGDITRQNYAFYVKGRFTISRDNSKNTLYLQMNSLRAEDTAVYYCNEMPPASTWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 5 aa, Artificial Sequence
  Synthetic Construct
INAMA

SEQ ID NO: 7 - protein, 19 aa, Artificial Sequence
  Synthetic Construct
GVTSSGSIVRENYAFYVKG

SEQ ID NO: 8 - protein, 6 aa, Artificial Sequence
  Synthetic Construct
IPPYST

SEQ ID NO: 9 - protein, 117 aa, Artificial Sequence
  Synthetic Construct
EVQLVESGGGLVKPGGSLRLSCAASGSTSSINAMAWYRQAPGKQREFVAGVTSSGSIVRENYAFYVKGRFTISRDNAKNSLYLQMNSLRAEDTAVYYCNAIPPYSTWGQGTQVTVSS

SEQ ID NO: 10 - protein, 344 aa, Artificial Sequence
  Synthetic Construct
EVQLVESGGGLVKPGGSLRLSCAASGSTSSINAMAWYRQAPGKQREFVAGVTSSGSIVRENYAFYVKGRFTISRDNAKNSLYLQMNSLRAEDTAVYYCNAIPPYSTWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 10 aa, Artificial Sequence
  Synthetic Construct
RASSSVSYIH

SEQ ID NO: 12 - protein, 7 aa, Artificial Sequence
  Synthetic Construct
ATSNLAS

SEQ ID NO: 13 - protein, 9 aa, Artificial Sequence
  Synthetic Construct
QQWTSNPPT

SEQ ID NO: 14 - protein, 5 aa, Artificial Sequence
  Synthetic Construct
SYNMH

SEQ ID NO: 15 - protein, 17 aa, Artificial Sequence
  Synthetic Construct
AIYPGNGDTSYNQKFKG

SEQ ID NO: 16 - protein, 12 aa, Artificial Sequence
  Synthetic Construct
STYYGGDWYFNV

SEQ ID NO: 17 - protein, 106 aa, Artificial Sequence [sequence omitted]

SEQ ID NO: 18 - protein, 121 aa, Artificial Sequence [sequence omitted]

SEQ ID NO: 19 - protein, 213 aa, Artificial Sequence
  Synthetic Construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 451 aa, Artificial Sequence
  Synthetic Construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 15 aa, Artificial Sequence
  Synthetic Construct
RASQSVSFAGTSLMH

SEQ ID NO: 22 - protein, 7 aa, Artificial Sequence
  Synthetic Construct
RASNLEA

SEQ ID NO: 23 - protein, 9 aa, Artificial Sequence
  Synthetic Construct
QQSREYPYT

SEQ ID NO: 24 - protein, 5 aa, Artificial Sequence
  Synthetic Construct
GYFMN

SEQ ID NO: 25 - protein, 17 aa, Artificial Sequence
  Synthetic Construct
RIHPYDGDTFYAQKFQG

SEQ ID NO: 26 - protein, 9 aa, Artificial Sequence
  Synthetic Construct
YDGSRAMDY

SEQ ID NO: 27 - protein, 111 aa, Artificial Sequence
  Synthetic Construct
DIVLTQSPASLAVSPGQRATITCRASQSVSFAGTSLMHWYQQKPGQPPKLLIYRASNLEAGVPARFSGSGSGTDFTLTINPVEANDAANYYCQQSREYPYTFGGGTKLEIK

SEQ ID NO: 28 - protein, 118 aa, Artificial Sequence
  Synthetic Construct
QVQLVQSGAEVKKPGASVKVSCKASGYTFTGYFMNWVRQAPGQGLEWIGRIHPYDGDTFYAQKFQGRVTMTRDTSTSTVYMELSSLRSEDTAVYYCTRYDGSRAMDYWGQGTTVTVSS

SEQ ID NO: 29 - protein, 218 aa, Artificial Sequence
  Synthetic Construct
DIVLTQSPASLAVSPGQRATITCRASQSVSFAGTSLMHWYQQKPGQPPKLLIYRASNLEAGVPARFSGSGSGTDFTLTINPVEANDAANYYCQQSREYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 448 aa, Artificial Sequence
  Synthetic Construct
QVQLVQSGAEVKKPGASVKVSCKASGYTFTGYFMNWVRQAPGQGLEWIGRIHPYDGDTFYAQKFQGRVTMTRDTSTSTVYMELSSLRSEDTAVYYCTRYDGSRAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 11 aa, Artificial Sequence
  Synthetic Construct
RASQDVNTAVA

SEQ ID NO: 32 - protein, 7 aa, Artificial Sequence
  Synthetic Construct
SASFLYS

SEQ ID NO: 33 - protein, 9 aa, Artificial Sequence
  Synthetic Construct
QQHYTTPPT

SEQ ID NO: 34 - protein, 5 aa, Artificial Sequence
  Synthetic Construct
DTYIH

SEQ ID NO: 35 - protein, 17 aa, Artificial Sequence
  Synthetic Construct
RIYPTNGYTRYADSVKG

SEQ ID NO: 36 - protein, 11 aa, Artificial Sequence
  Synthetic Construct
WGGDGFYAMDY

SEQ ID NO: 37 - protein, 107 aa, Artificial Sequence [sequence omitted]

SEQ ID NO: 38 - protein, 120 aa, Artificial Sequence [sequence omitted]

SEQ ID NO: 39 - protein, 214 aa, Artificial Sequence
  Synthetic Construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 450 aa, Artificial Sequence
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 5 aa, Artificial Sequence
  Synthetic Construct
VNAYG

SEQ ID NO: 42 - protein, 16 aa, Artificial Sequence
  Synthetic Construct
IISAGGTTNYADSVKG

SEQ ID NO: 43 - protein, 10 aa, Artificial Sequence
  Synthetic Construct
QRRIGMLRDY

SEQ ID NO: 44 - protein, 118 aa, Artificial Sequence
  Synthetic Construct
QVQLVESGGGLVQPGGSLRLSCAASGITFPVNAYGWYRQAPGKQRDLVAIISAGGTTNYADSVKGRFTISRDNSKNTLYLQMNSLRAEDTAVYYCYLQRRIGMLRDYWGQGTQVTVSS

SEQ ID NO: 45 - protein, 345 aa, Artificial Sequence
  Synthetic Construct
QVQLVESGGGLVQPGGSLRLSCAASGITFPVNAYGWYRQAPGKQRDLVAIISAGGTTNYADSVKGRFTISRDNSKNTLYLQMNSLRAEDTAVYYCYLQRRIGMLRDYWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 5 aa, Artificial Sequence
  Synthetic Construct
SYVMA

SEQ ID NO: 47 - protein, 17 aa, Artificial Sequence
  Synthetic Construct
SINWSSGRLIYADSVKG

SEQ ID NO: 48 - protein, 112 aa, Artificial Sequence
  Synthetic Construct
QVQLVESGGGLVQPGGSLRLSCAASGRTLESYVMAWFRQAPGKEREAVASINWSSGRLIYADSVKGRFTISRDNSKNTLYLQMNSLRAEDTAVYYCAAGRYWGQGTQVTVSS

SEQ ID NO: 49 - protein, 339 aa, Artificial Sequence
  Synthetic Construct
QVQLVESGGGLVQPGGSLRLSCAASGRTLESYVMAWFRQAPGKEREAVASINWSSGRLIYADSVKGRFTISRDNSKNTLYLQMNSLRAEDTAVYYCAAGRYWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 11 aa, Artificial Sequence
  Synthetic Construct
KASQDVSIAVA

SEQ ID NO: 51 - protein, 7 aa, Artificial Sequence
  Synthetic Construct
SASYRYT

SEQ ID NO: 52 - protein, 9 aa, Artificial Sequence
  Synthetic Construct
QQHYITPLT

SEQ ID NO: 53 - protein, 5 aa, Artificial Sequence
  Synthetic Construct
NYGMN

SEQ ID NO: 54 - protein, 17 aa, Artificial Sequence
  Synthetic Construct
WINTYTGEPTYTDDFKG

SEQ ID NO: 55 - protein, 12 aa, Artificial Sequence
  Synthetic Construct
GGFGSSYWYFDV

SEQ ID NO: 56 - protein, 107 aa, Artificial Sequence
  Synthetic Construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 57 - protein, 121 aa, Artificial Sequence
  Synthetic Construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 58 - protein, 214 aa, Artificial Sequence
  Synthetic Construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 451 aa, Artificial Sequence
  Synthetic Construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK